(19) United States
(12) Patent Application Publication
(10) Pub. No.: US 2026/0365642 A1
(43) Pub. Date: Nov. 19, 2026

(54) ANTIPARASITIC COMPOUNDS

(71) Applicant: Animol Discovery, Inc., Boston, MA (US)

(72) Inventors: Nigel Walshe, Canterbury (GB); Andrew Antony Calabrese, Cockeysville, MD (US)

(21) Appl. No.: 19/700,979

(22) Filed: Jun. 8, 2026

Related U.S. Application Data
(63) Continuation of application No. PCT/IB2024/062364, filed on Dec. 8, 2024.
(60) Provisional application No. 63/607,605, filed on Dec. 8, 2023.

(51) Int. Cl. C07D 413/14; C07D 401/04; C07D 401/14; C07D 417/14; C07D 487/10; C07D 498/04

(52) U.S. Cl. CPC C07D 413/14; C07D 401/04; C07D 401/14; C07D 417/14; C07D 487/10; C07D 498/04

(57) ABSTRACT

Compounds exhibiting activity against various parasites are provided herein, along with compositions containing such compounds. The disclosure further provides methods of preventing and/or treating parasitic infections and/or infestations in animals by administering such compounds or compositions to the animals.

### CROSS-REFERENCE TO RELATED APPLICATIONS

This application is a continuation of International Application No. PCT/IB2024/062364, filed Dec. 8, 2024, which claims the benefit of U.S. Provisional Patent Application No. 63/607,605, filed Dec. 8, 2023, the disclosures of each which are incorporated herein by reference in their entirety.

### FIELD OF THE DISCLOSURE

The present disclosure relates to compounds, compositions, and methods of use thereof, e.g., in the prevention and/or treatment of parasites in human and animal health.

### BACKGROUND

Parasites, which live on or within host organisms, can adversely affect animals, humans, and plants. Parasites include, e.g., protozoa, helminths, arthropods, and fungi. Parasites are a significant concern in veterinary medicine, often causing irritation/discomfort to animals and, if left untreated, leading to harm and, in some cases, death. Parasites include both endoparasites (living within an animal's body) such as whipworm, hookworm, roundworm, and heartworm and ectoparasites (living on the surface of an animal's body) such as fleas, ticks, and mites. Parasites may adversely affect the health of the animal and can have further reaching effects. For example, many parasites are vectors of pathogenic agents that can affect both animals and humans (including, e.g., ticks that may transmit Lyme disease and Rocky Mountain spotted fever, fleas that may transmit tapeworm, and mosquitos that may transmit malaria). As another example of the further reaching effects of parasites, parasitic infestations in livestock can lead to adverse economic impacts e.g., by interfering with animal digestion and nutrient absorption as well as causing damage to tissue and organs. Such impacts on infected livestock can lead to poor production performance as manifested by little or no weight gain, metabolic disturbances, reproductive abnormalities, reduced milk production and/or quality, and death of the animal, all of which result in economic loss to farmers). Some parasites (e.g., certain helminths, arthropods, and fungi) cause damage to plants, threatening food crop production and contaminating fruits and vegetables, leading to economic as well as public health concerns.

Various compounds have been developed as antiparasitic agents against one or more parasites. However, certain such compounds suffer from one or more drawbacks, e.g., insufficient antiparasitic efficacy, slow onset of action, limited duration of action, high cost of goods, or combinations thereof. Some such compounds have a low therapeutic index and require significant care in storing and administering to animals. Some antiparasitic agents are becoming ineffective due to development of resistance in parasites. For at least these reasons, there is continuing interest in the art to develop additional compounds capable of eliminating or controlling various parasites, and which can be used to prevent or treat certain infections, infestations, and/or diseases associated with or transmitted by such parasites.

### BRIEF SUMMARY

The present disclosure relates to compounds, pharmaceutical compositions, and methods of treating and/or preventing parasitic infection and/or infestation in animals and/or humans. The compounds of the disclosure possess a 4,6-diaminopyrimidine core structure and exhibit activity against endoparasites and/or ectoparasites. In some embodiments, compounds of the disclosure exhibit anthelmintic activity at the larval stage of a parasite, for example, against Dirofilaria immitis (heartworm).

The disclosure provides, without limitation, the following embodiments:

Embodiment 1: A compound having a structure according to Formula I:

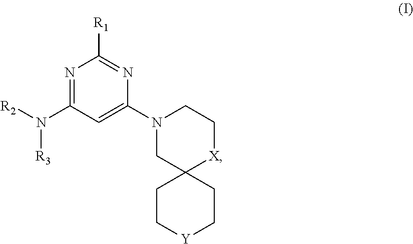

or a pharmaceutically acceptable salt, wherein:
R₁ is H or C₁-C₆ alkyl, wherein said C₁-C₆ alkyl is optionally substituted with one or more F atoms;
X is CH₂, O, S, or SO₂;
Y is a bond or is CR₄R₅;
R₂ is H, C₁-C₆ alkyl, benzyl, or (CH₂)ₙNMe₂;
R₃ is (CH₂)ₘ—R₆;
or R₂ and R₃, together with the nitrogen atom to which they are attached, form a 4 to 11 membered ring system, which may be monocyclic, bicyclic, or spiro-fused, wherein said ring system may further comprise one or more additional heteroatoms selected from O, N, and S, and wherein said ring system may optionally be substituted;
R₄ and R₅ are each independently selected from H, F, and CH₃;
R₆ is selected from the group consisting of:

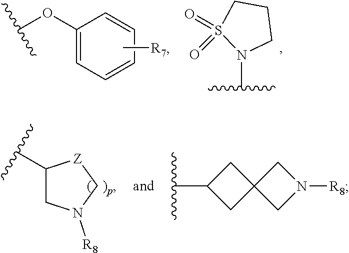

Z is CH₂, CF₂, O, S, or SO₂;
R₇ is H, halogen, OH, NH₂, C₁-C₆ alkyl, —O—C₁-C₆ alkyl, —NR₁—C₁-C₆ alkyl, —S—C₁-C₆ alkyl, or —SO₂—C₁-C₆ alkyl, wherein each said C₁-C₆ alkyl is optionally substituted with one or more F atoms;
R₈ is CO₂CH₃, SO₂R₉, or SO₂NMe₂;
R₉ is C₁-C₆ alkyl or C₃-C₆ cycloalkyl;
n is 1, 2, or 3;
m is 0, 1, 2, or 3; and
p is an integer from 0 to 3.

Embodiment 2: The compound of Embodiment 1, wherein Y is CH₂.

Embodiment 3: The compound of Embodiment 1, wherein Y is CF₂.

Embodiment 4: The compound of any of Embodiments 1-3, wherein X is CH₂.

Embodiment 5: The compound of any of Embodiments 1-4, wherein X is O.

Embodiment 6: The compound of any of Embodiments 1-4, wherein X is S.

Embodiment 7: The compound of any of Embodiments 1-4, wherein X is SO₂.

Embodiment 8: The compound of any of Embodiments 1-7, wherein R₁ is CH₃ or CF₃.

Embodiment 9: The compound of any of Embodiments 1-8, wherein R₂ is Me or Et.

Embodiment 10: The compound of any of Embodiments 1-8, wherein R₂ is Bn.

Embodiment 11: The compound of any of Embodiments 1-8, wherein R₂ is (CH₂)₂NMe₂.

Embodiment 12: The compound of any of Embodiments 1-11, wherein R₃ is —(CH₂)₂OPh.

Embodiment 13: The compound of any of Embodiments 1-11, wherein R₃ is

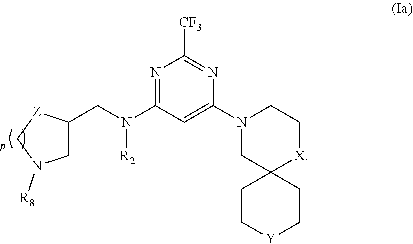

Embodiment 14: The compound of any of Embodiments 1-13, wherein R₆ is

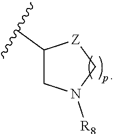

Embodiment 15: The compound of any of Embodiments 1-14, wherein the compound of Formula I has a structure according to Formula Ia:

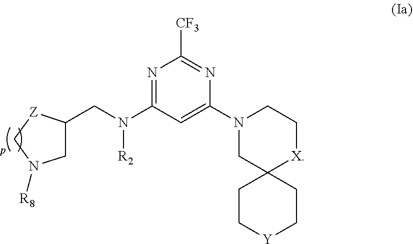

Embodiment 16: The compound of Embodiment 15, wherein Z is O, S, SO₂, CH₂, or CF₂; and p is 0, 1, 2 or 3, provided that when Z is O, S, or SO₂, p is not 0 or 1.

Embodiment 17: The compound of any of Embodiments 15-16, wherein, Z is O.

Embodiment 18: The compound of any of Embodiments 15-16, wherein Z is CH₂, and p is 0, 1, or 2.

Embodiment 19: The compound of any of Embodiments 15-18, wherein R₈ is CO₂Me.

Embodiment 20: The compound of any of Embodiments 15-18, wherein R₈ is SO₂NMe₂.

Embodiment 21: The compound of any of Embodiments 15-18, wherein R₈ is SO₂R₉, and R₉ is Me, Et, or cyclopropyl.

Embodiment 22: The compound of any of Embodiments 1-14, wherein the compound of Formula I has a structure according to Formula Ib:

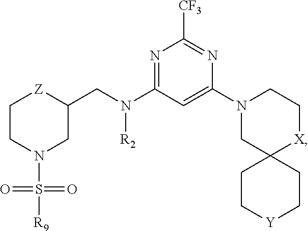

wherein:
R₂ is Me, Et, or CH₂CH₂NMe₂;
X is CH₂, CF₂, O, S, or SO₂;
Y is CH₂ or CF₂; and
Z is O, S, SO₂, or CF₂;
R₉ is Me, Et, or cyclopropyl.

Embodiment 23: The compound of Embodiment 22, wherein the compound of Formula I has a structure according to Formula Ib wherein:
X is CH₂, CF₂, O, S;
Y is CH₂; and
Z is O, S, or CF₂.

Embodiment 24: The compound of Embodiment 22 or 23, wherein the compound has in vitro antiparasitic activity toward Dirofilaria immitis at a concentration of about 10 mM or less, or about 1 mM or less.

Embodiment 25: The compound of Embodiment 22 or 23, wherein the compound of Formula Ib has an IC₅₀ for inhibition of C. elegans acetyl-CoA carboxylase (ACCase) of about 5 mM or less, about 1 mM or less, or about 500 nM or less.

Embodiment 26: The compound of any one of Embodiments 22-25, wherein the compound has an IC₅₀ is in a range from about 1 nM to about 1 mM.

Embodiment 27: The compound of any one of Embodiments 15-21, wherein the compound has a structure according to Formula Ia wherein:
p is 3;
X is CH₂, CF₂, O, or S;
Y is CH₂;
Z is O;
R₂ is Me or Et; and
R₉ is Me, Et, or cyclopropyl.

Embodiment 28: The compound of Embodiment 27, wherein the compound of Formula Ia has in vitro antiparasitic activity toward Dirofilaria immitis at a concentration of about 10 mM or less, or about 1 mM or less.

Embodiment 29: The compound of Embodiment 27 or 28, wherein the compound of Formula Ia has an IC₅₀ for inhibition of C. elegans acetyl-CoA carboxylase (ACCase) of about 5 mM or less, about 1 mM or less, or about 500 nM or less.

Embodiment 30: The compound of any of Embodiments 15-21, wherein the compound has a structure according to Formula Ia wherein:
p is 0;
X, Y, and Z are CH₂;
R₂ is Me or Et;
R₈ is SO₂R₉; and
R₉ is Me or Et.

Embodiment 31: The compound of Embodiment 30, wherein the compound of Formula Ia has vitro antiparasitic activity toward Dirofilaria immitis at a concentration of about 10 mM or less, or about 1 mM or less.

Embodiment 32: The compound of any of Embodiments 15-21, wherein the compound has a structure according to Formula Ia wherein:
X is O;
Y is CH₂ or CF₂;
Z is O;
R₂ is Me or Et; and
R₉ is Me, Et, or cyclopropyl;
wherein the compound has in vitro antiparasitic activity toward Dirofilaria immitis at a concentration of about 10 mM or less, or about 1 mM or less.

Embodiment 33: The compound of any of Embodiments 15-21, wherein the compound has a structure according to Formula Ia wherein:
p is 2 or 3;
X is CH₂, CF₂, or O;
Y is CH₂ or CF₂;
Z is O CF₂, or SO₂;
R₂ is Me; and
R₉ is Me;
wherein the compound has in vitro antiparasitic activity toward Dirofilaria immitis at a concentration in a range from about 30 mM to about 100 mM.

Embodiment 34: The compound of any of Embodiments 1-8, wherein R₂ and R₃, together with the nitrogen atom to which they are attached, form a 4 to 11 membered bicyclic ring system, the mono- or bicyclic ring system optionally comprising one or more heteroatoms selected from O, N, and S.

Embodiment 35: The compound of any of Embodiments 1-8, wherein R₂ and R₃, together with the nitrogen atom to which they are attached, form a 7 to 11 membered spiro-fused ring system, the spiro-fused ring system optionally comprising one or more heteroatoms selected from O, N, and S.

Embodiment 36: A composition comprising a compound of Formula I or a compound of any one of Embodiments 1-35 or a pharmaceutically acceptable salt thereof, and a pharmaceutically or veterinarily acceptable excipient.

Embodiment 37: A method of treating or preventing a parasitic infection or infestation in an animal, the method comprising administering to the animal an effective amount of a compound of Formula I or a compound of any one of Embodiments 1-35, a pharmaceutically or veterinarily acceptable salt thereof, or a composition comprising said compound or salt thereof, e.g., the composition of Embodiment 36.

These and other features, aspects, and advantages of the present disclosure will be apparent from a reading of the following detailed description. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise. It will therefore be appreciated that this Summary is provided merely for purposes of summarizing some example implementations so as to provide a basic understanding of some aspects of the disclosure. Accordingly, it will be appreciated that the above-described example implementations are merely examples and should not be construed to narrow the scope or spirit of the disclosure in any way. Other example implementations, aspects, and advantages will become apparent from the following detailed description.

### DETAILED DESCRIPTION

The present disclosure will now be described more fully hereinafter with reference to example embodiments thereof. Before describing several example embodiments of the technology, it is to be understood that the technology is not limited to the details of construction or process steps set forth in the following description. The technology is capable of other embodiments and of being practiced or being carried out in various ways.

The following description sets forth numerous exemplary configurations, methods, parameters, and the like in order to provide a thorough understanding of various embodiments of the disclosure. It should be recognized, however, that such description is not intended as a limitation on the scope of the present disclosure but is instead provided as a description of exemplary embodiments.

The disclosure provides certain compounds, methods of preparing such compounds, compositions comprising such compounds (alone or in combination with one or more additional active agents), and methods of use associated with such compounds and/or compositions. In particular, the disclosure provides compounds with activity against endoparasites and/or ectoparasites, compositions comprising such compounds or pharmaceutically or veterinarily acceptable salts thereof, and methods of treating or preventing certain parasitic infections utilizing such compounds or compositions, optionally ion combination with other active agents. In some embodiments, compounds of the disclosure exhibit anthelmintic activity at the larval stage of the parasite, for example, against Dirofilaria immitis (heartworm).

It is noted that the focus of this application is on compounds for use in animal and human health, but it is to be understood that such compounds may have other applications (e.g., including, but not limited to, use in an agricultural context).

### DEFINITIONS

Unless defined otherwise, all technical and scientific terms used herein have the same meaning as is commonly understood by one of skill in the art to which this disclosure belongs. With respect to the terms used in this disclosure, the following definitions are provided. This application will use the following terms as defined below unless the context of the text in which the term appears requires a different meaning.

The articles “a” and “an” as used in this disclosure may refer to one or more than one (i.e., to at least one) of the grammatical object of the article. By way of example, “an element” may mean one element or more than one element.

As used herein, the term “and/or” as used in this disclosure may mean either “and” or “or” unless indicated otherwise.

The term “about” used throughout this specification is used to describe and account for small fluctuations. For example, the term “about” can refer to less than or equal to ±10%, less than or equal to ±5%, less than or equal to ±2%, less than or equal to +1%, less than or equal to ±0.5%, less than or equal to ±0.2%, less than or equal to ±0.1% or less than or equal to ±0.05%. All numeric values herein are modified by the term “about,” whether or not explicitly indicated. A value modified by the term “about” of course includes the specific value. For instance, “about 5.0” must include 5.0.

Unless the context requires otherwise, throughout the present specification and claims, as used herein, the terms “including,” “containing,” and “comprising” are used in their open, non-limiting sense.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆ alkylene- may be selected from methylene, ethylene, propylene, butylene, pentylene, and hexylene, any one of which is optionally substituted.

“Alkoxy” refers to a radical bonded through an oxygen atom of the formula -o-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

The terms “C_{x-y} alkenyl” and “C_{x-y} alkynyl” refer to substituted or unsubstituted unsaturated aliphatic groups analogous in length and possible substitution to the alkyls described above, but that contain at least one double or triple bond, respectively. The term —C_{x-y} alkenylene- refers to a substituted or unsubstituted alkenylene chain with from x to y carbons in the alkenylene chain. For example, —C₂₋₆ alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y} alkynylene- refers to a substituted or unsubstituted alkynylene chain with from x to y carbons in the alkenylene chain. For example, —C₂₋₆ alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atoms (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atoms (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Aralkyl” refers to a radical of the formula —R^{c}-aryl where R^{c} is an alkylene chain as defined above, for example, methylene, ethylene, and the like.

“Aralkenyl” refers to a radical of the formula —R^{d}-aryl where R^{d} is an alkenylene chain as defined above. “Aralkynyl” refers to a radical of the formula —R^{e}-aryl, where R^{e} is an alkynylene chain as defined above.

“Carbocycle” refers to a saturated, unsaturated or aromatic ring in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

“Cycloalkylalkyl” refers to a radical of the formula —R^{c}-cycloalkyl where R^{c} is an alkylene chain as described above.

“Cycloalkylalkoxy” refers to a radical bonded through an oxygen atom of the formula —O—R^{c}-cycloalkyl where R^{c} is an alkylene chain as described above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Heterocycle” refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. In some embodiments, the heterocycle is a heteroaryl. In some embodiments, the heterocycle is a heterocycloalkyl. “Heterocyclene” refers to a divalent heterocycle linking the rest of the molecule to a radical group

“Heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heterocycloalkylalkyl” refers to a radical of the formula —R^{c}-heterocycloalkyl where R^{c} is an alkylene chain as defined above. If the heterocycloalkyl is a nitrogen-containing heterocycloalkyl, the heterocycloalkyl is optionally attached to the alkylene chain at the nitrogen atom.

“Heteroaryl” or “aromatic heterocycle” refers to a radical derived from a 3- to 12-membered aromatic ring radical that comprises one to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) it-electron system in accordance with the Hickel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkylene chain at the nitrogen atom.

The term “bridged” as used herein refers to bicyclic ring systems (e.g., cycloalkyl or heterocyclyl) in which two bridgehead atoms are separated by a bridge atom, and two rings share three or more atoms (the bridgehead and bridge atoms).

The term “amino” as used herein refers to —NH₂.

The terms “hydroxy” and “hydroxyl” refer to —OH.

The term “oxo” as used herein refers to an “═O” group. It can also be abbreviated herein as C(O) or as C═O.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

As used herein, the term “unsubstituted” means that the specified group bears no substituents beyond the moiety recited (e.g., where valency is satisfied by hydrogen).

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents or resolved using conventional techniques. The optical activity of a compound can be analyzed via any suitable method, including but not limited to chiral chromatography and polarimetry, and the degree of predominance of one stereoisomer over the other isomer can be determined.

In certain embodiments, the compounds of the disclosure may contain asymmetric or chiral centers, and, therefore, exist in different stereoisomeric forms. The term “stereoisomers” may refer to the set of compounds which have the same number and type of atoms and share the same bond connectivity between those atoms but differ in three-dimensional structure. The term “stereoisomer” may refer to any member of this set of compounds. For instance, a stereoisomer may be an enantiomer or a diastereomer. It is intended that all stereoisomeric forms of the compounds of the disclosure as well as mixtures thereof, including racemic mixtures, form part of the present disclosure.

When stereochemistry is not specified, certain molecules described herein include isomers, such as enantiomers and diastereomers, mixtures of enantiomers, including racemates, mixtures of diastereomers, and other mixtures thereof, to the extent they can be made by one of ordinary skill in the art by routine experimentation. In certain embodiments, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration.

In certain embodiments, chiral centers in compounds of the present disclosure may have the S or R configuration as defined by the IUPAC 1974 Recommendations.

The term “radical of a compound” as used herein refers to a structure derived from a parent compound by removal of one or more atoms, e.g., hydrogen atoms. In one embodiment, a “radical of a compound” is a monovalent radical derived from the removal of one hydrogen atom from the parent compound.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” and the like.

Wherever a substituent is depicted as a di-radical (i.e., has two points of attachment to the rest of the molecule), it is to be understood that the substituent can be attached in any directional configuration unless otherwise indicated.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

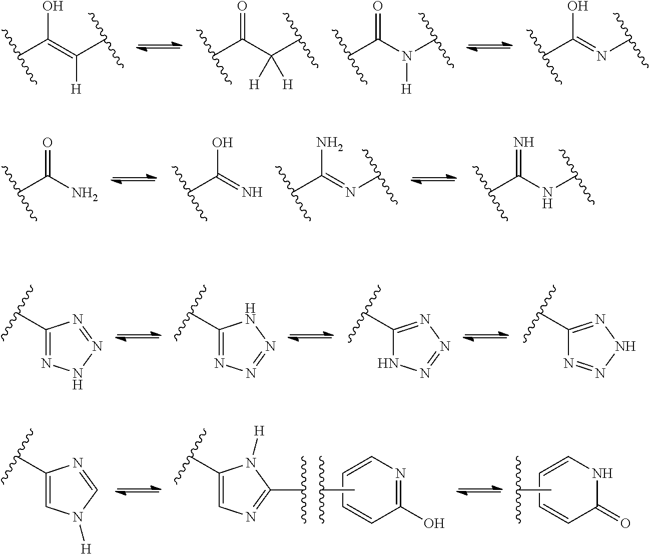

“Stable compound” and “stable structure” may indicate a compound that is sufficiently robust to survive isolation to a useful degree of purity from a reaction mixture, and formulation into an efficacious therapeutic agent.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “carrier,” as used in this disclosure, may encompass carriers, excipients, and diluents and may mean a material, composition or vehicle, such as a liquid or solid filler, diluent, excipient, solvent or encapsulating material, involved in carrying or transporting a pharmaceutical agent, such as one or more compounds, or pharmaceutically acceptable salts, solvates (e.g., hydrates), isomers (e.g., stereoisomers), and tautomers thereof, of the disclosure, from one organ, or portion of the body, to another organ, or portion of the body of a subject. Carriers should be selected on the basis of compatibility and the release profile properties of the desired dosage form. Exemplary carrier materials may include, e.g., adjuvants, binders, suspending agents, disintegration agents, filling agents, surfactants, solubilizers, stabilizers, lubricants, wetting agents, diluents, spray-dried dispersions, and the like. See, e.g., Hoover, John E., Remington's Pharmaceutical Sciences, Mack Publishing Co., Easton, Pa. 1975. Exemplary carrier materials may also include without limitation any adjuvant, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The terms “pharmaceutically acceptable” or “pharmacologically acceptable” may refer to a material which is not biologically, or otherwise, undesirable—the material may be administered to an individual without causing any substantially undesirable biological effects or interacting in a deleterious manner with any of the components of the composition in which it is contained.

A “pharmaceutical composition” may refer to a formulation of a compound of the disclosure and a medium generally accepted in the art for the delivery of the biologically active compound to a subject, e.g., mammals or humans. Such a medium may include all pharmaceutically acceptable carriers therefor.

The terms “subject,” “individual,” and “patient” may be used interchangeably and refer to non-human mammals (e.g., non-human primates, canines, equines, felines, porcines, bovines, ungulates, lagomorphs, and the like).

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used.

The term “effective amount” or “therapeutically effective amount” refers to that amount of a compound or salt described herein that is sufficient to affect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. In some embodiments, a therapeutic benefit may include alleviating, abating or ameliorating symptoms of a disease or condition, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In some embodiments, the therapeutic benefit includes the eradication or amelioration of the underlying disorder being treated. In some embodiments, the therapeutic benefit includes the the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compounds or compositions are administered to a subject at risk of developing a particular disease, or to a subject exhibiting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample. In some embodiments, preventing comprises delaying or precluding the onset, recurrence, or spread (in whole or in part) of a disorder, disease, or condition; barring a subject from acquiring a disorder, disease, or condition; or reducing a subject's risk of acquiring a disorder, disease, or condition.

In certain embodiments, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

### Compounds of the Disclosure

The present disclosure provides compounds which are biologically active against parasites. As such, in some embodiments, compounds of the formula(s) disclosed herein can be referred to as “parasiticidal compounds” of the relevant formula(s).

In particular, the present disclosure provides compounds having a structure according to Formula I:

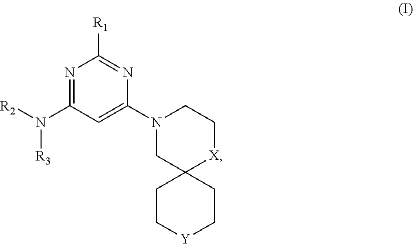

or a pharmaceutically acceptable salt, wherein:
R₁ is H or C₁-C₆ alkyl, wherein said C₁-C₆ alkyl is optionally substituted with one or more F atoms;
X is CH₂, O, S, or SO₂;
Y is a bond or is CR₄R₅;
R₂ is H, C₁-C₆ alkyl, benzyl, or (CH₂)ₙNMe₂;
R₃ is (CH₂)ₘ—R₆;
or R₂ and R₃, together with the nitrogen atom to which they are attached, form a 4 to 11 membered ring system, which may be monocyclic, bicyclic, spiro-fused, or bridged, wherein said ring system may further comprise one or more additional heteroatoms selected from O, N, and S, and wherein said ring system may optionally be substituted;
R₄ and R₅ are each independently selected from H, F, and CH₃;
R₆ is selected from the group consisting of

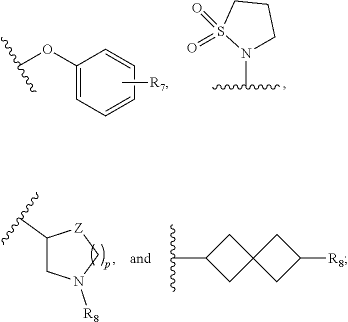

Z is CH₂, CF₂, O, S, or SO₂;
R₇ is H, halogen, OH, NH₂, C₁-C₆ alkyl, —O—C₁-C₆ alkyl, —NR₁—C₁-C₆ alkyl, —S—C₁-C₆ alkyl, or —SO₂—C₁-C₆ alkyl, wherein each said C₁-C₆ alkyl is optionally substituted with one or more F atoms;
R₈ is CO₂CH₃, SO₂R₉, or SO₂NMe₂;
R₉ is C₁-C₆ alkyl or C₃-C₆ cycloalkyl;
n is 1, 2, or 3;
m is 0, 1, 2, or 3; and
p is an integer from 0 to 3.

In some embodiments, X is CH₂. In some embodiments, X is O. In some embodiments, X is S. In some embodiments, X is SO₂.

In some embodiments, Y is a bond. In some embodiments, Y is CH₂ or CF₂.

In some embodiments, X is CH₂ and Y is a bond. In some embodiments, X is CH₂ and Y is CH₂. In some embodiments, X is CH₂ and Y is CF₂. In some embodiments, X is CH₂ and Y is C(CH₃)₂.

In some embodiments, X is O and Y is a bond. In some embodiments, X is O and Y is CH₂. In some embodiments, X is O and Y is CF₂. In some embodiments, X is O and Y is C(CH₃)₂.

In some embodiments, X is S and Y is a bond. In some embodiments, X is S and Y is CH₂. In some embodiments, X is S and Y is CF₂. In some embodiments, X is S and Y is C(CH₃)₂.

In some embodiments, X is SO₂ and Y is a bond. In some embodiments, X is SO₂ and Y is CH₂. In some embodiments, X is SO₂ and Y is CF₂. In some embodiments, X is SO₂ and Y is C(CH₃)₂.

In some embodiments, X is CF₂ and Y is a bond. In some embodiments, X is CF₂ and Y is CH₂. In some embodiments, X is CF₂ and Y is CF₂. In some embodiments, X is CF₂ and Y is C(CH₃)₂.

In some embodiments, R₁ is CH₃. In some embodiments, R₁ is CF₃.

In some embodiments, R₂ is H. In some embodiments, R₂ is CH₃. In some embodiments, R₂ is Et. In some embodiments, R₂ is Bn. In some embodiments, R₂ is (CH₂)₂NMe₂.

In some embodiments, R₃ is (CH₂)ₘ—R₆, wherein R₆ is

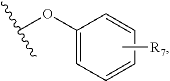

and wherein R₇ is R₇ is H, halogen, OH, NH₂, C₁-C₆ alkyl, —O—C₁-C₆ alkyl, —NR₁—C₁-C₆ alkyl, —S—C₁-C₆ alkyl, or —SO₂—C₁-C₆ alkyl, wherein each said C₁-C₆ alkyl is optionally substituted with one or more F atoms.

In some such embodiments, m is 2 or 3.

In some embodiments, R₃ is (CH₂)ₘ—R₆, wherein R₆ is

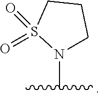 [SMILES: *N1CCCS1(=O)=O]

In some such embodiments, m is 2 or 3.

In some embodiments, R₃ is (CH₂)ₘ—R₆, wherein R₆ is

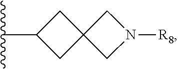

wherein R₈ is CO₂CH₃, SO₂R₉, or SO₂NMe₂, and wherein R₉ is C₁-C₆ alkyl or C₃-C₆ cycloalkyl. In some such embodiments, m is 0. In some such embodiments, m is 1. In some such embodiments, m is 2. In some such embodiments, m is 3.

In some embodiments, R₃ is (CH₂)ₘ—R₆, wherein:
R₆ is

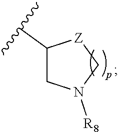

R₈ is CO₂CH₃, SO₂R₉, or SO₂NMe₂;
R₉ is C₁-C₆ alkyl or C₃-C₆ cycloalkyl;
m is 0, 1, 2, or 3; and
p is 0, 1, 2, or 3.

In some embodiments, the compound of Formula I has a structure according to Formula Ia:

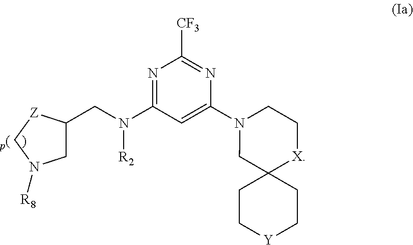

wherein each of X, Y, Z, R₂, R₈, and p are as defined herein above.

In some embodiments, the compound has a structure according to Formula Ia, wherein Z is O, S, SO₂, CH₂, or CF₂, and p is 0, 1, 2 or 3, provided that when Z is O, S, or SO₂, p is not 0 or 1.

In some embodiments, the compound has a structure according to Formula Ia, wherein Z is O and p is 2 or 3.

In some embodiments, the compound has a structure according to Formula Ia, wherein Z is CH₂ and p is 0, 1, or 2.

In some embodiments, the compound has a structure according to Formula Ia, wherein R₈ is CO₂Me.

In some embodiments, the compound has a structure according to Formula Ia, wherein R₈ is SO₂NMe₂.

In some embodiments, the compound has a structure according to Formula Ia, wherein R₈ is SO₂R₉, and R₉ is Me, Et, or cyclopropyl.

In some embodiments, the compound has a structure according to Formula Ia, wherein X is CH₂, CF₂, O, S; Y is CH₂; and Z is O, S, or CF₂.

In some embodiments, the compound has a structure according to Formula Ia, wherein:
p is 3;
X is CH₂, CF₂, O, S, or SO₂;
Y is CH₂;
Z is O;
R₂ is Me or Et;
R₈ is SO₂R₉; and
R₉ is Me, Et, or cyclopropyl.

In some embodiments, the compound has a structure according to Formula Ia, wherein:
p is 3;
X is CH₂, CF₂, O, S, or SO₂;
Y is CH₂ or CF₂;
Z is CH₂, CF₂, O, S, or SO₂;
R₂ is Me or Et;
R₈ is SO₂R₉; and
R₉ is Me, Et, or cyclopropyl.

In some embodiments, the compound has a structure according to Formula Ia, wherein:
p is 3;
X is CH₂, CF₂, O, or S;
Y is CH₂;
Z is O;
R₂ is Me or Et;
R₈ is SO₂R₉; and
R₉ is Me, Et, or cyclopropyl.

In some embodiments, the compound has a structure according to Formula Ia, wherein:
p is 0;
X is CH₂, CF₂, O, S, or SO₂;
Y is CH₂ or CF₂;
Z is CH₂, CF₂, O, S, or SO₂;
R₂ is Me or Et;
R₈ is SO₂R₉; and
R₉ is Me or Et.

In some embodiments, the compound has a structure according to Formula Ia, wherein:
p is 0;
X, Y, and Z are CH₂;
R₂ is Me or Et;
R₈ is SO₂R₉; and
R₉ is Me or Et.

In some embodiments, the compound has a structure according to Formula Ia, wherein:
p is 2 or 3;
X is CH₂, CF₂, or O;
Y is CH₂ or CF₂;
Z is O CF₂, or SO₂;
R₂ is Me; and
R₉ is Me;
In some embodiments, the compound of Formula I has a structure according to Formula Ib:

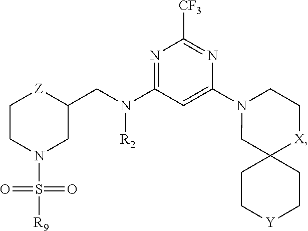

wherein:
X is CH₂, CF₂, O, S, or SO₂;
Y is CH₂ or CF₂;
Z is O, S, SO₂, or CF₂;
R₂ is Me, Et, or CH₂CH₂NMe₂; and
R₉ is Me, Et, or cyclopropyl.

In some embodiments, the compound has a structure according to Formula Ib, wherein:
R₂ is Me, Et, or CH₂CH₂NMe₂;
X is CH₂, CF₂, O, S, or SO₂;
Y is CH₂ or CF₂; and
Z is O, S, SO₂, or CF₂;
R₉ is Me, Et, or cyclopropyl.

In some embodiments, the compound has a structure according to Formula Ib, wherein:
X is CH₂, CF₂, O, S;
Y is CH₂; and
Z is O, S, or CF₂.

In some embodiments, the compound has a structure according to Formula Ib, wherein:
X is O;
Y is CH₂ or CF₂;
Z is O;
R₂ is Me or Et; and
R₉ is Me, Et, or cyclopropyl.

In some embodiments, the compound has a structure according to Formula Ib, wherein X and Y are each CH₂.

In some embodiments, the compound having a structure according to Formula Ib has a structure selected from the group consisting of

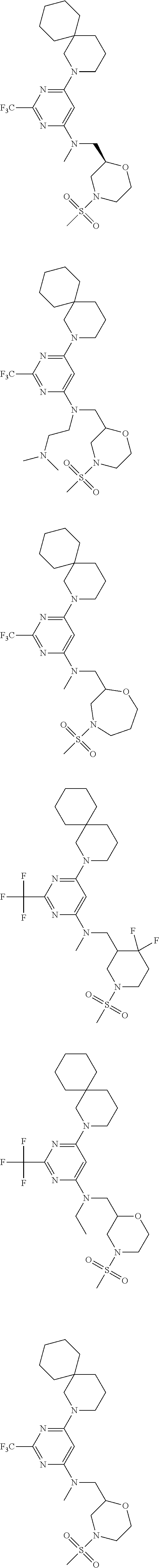 [SMILES: CN(C[C@@H]1CN(S(C)(=O)=O)CCO1)c1cc(N2CCCC3(CCCCC3)C2)nc(C(F)(F)F)n1 ; CN(C)CCN(CC1CN(S(C)(=O)=O)CCO1)c1cc(N2CCCC3(CCCCC3)C2)nc(C(F)(F)F)n1 ; CN(CC1CN(S(C)(=O)=O)CCCO1)c1cc(N2CCCC3(CCCCC3)C2)nc(C(F)(F)F)n1 ; CN(CC1CN(S(C)(=O)=O)CCC1(F)F)c1cc(N2CCCC3(CCCCC3)C2)nc(C(F)(F)F)n1 ; CCN(CC1CN(S(C)(=O)=O)CCO1)c1cc(N2CCCC3(CCCCC3)C2)nc(C(F)(F)F)n1 ; CN(CC1CN(S(C)(=O)=O)CCO1)c1cc(N2CCCC3(CCCCC3)C2)nc(C(F)(F)F)n1]

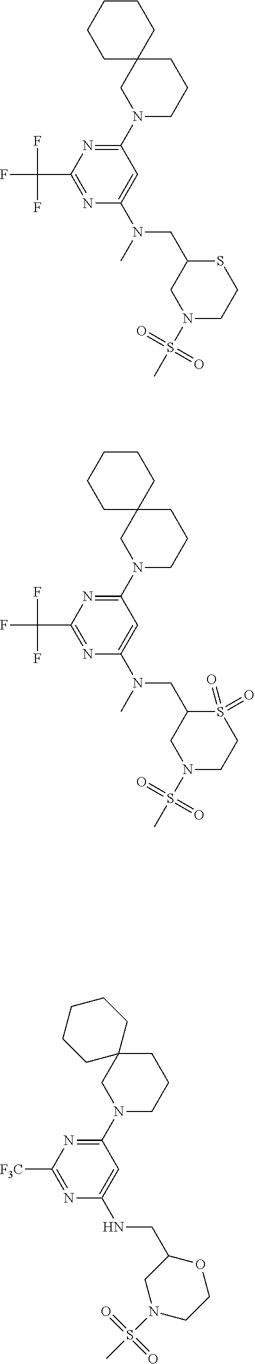 [SMILES: CN(CC1CN(S(C)(=O)=O)CCS1)c1cc(N2CCCC3(CCCCC3)C2)nc(C(F)(F)F)n1 ; CN(CC1CN(S(C)(=O)=O)CCS1(=O)=O)c1cc(N2CCCC3(CCCCC3)C2)nc(C(F)(F)F)n1 ; CS(=O)(=O)N1CCOC(CNc2cc(N3CCCC4(CCCCC4)C3)nc(C(F)(F)F)n2)C1]

In some embodiments, the compound having a structure according to Formula Ib has a structure selected from the group consisting of:

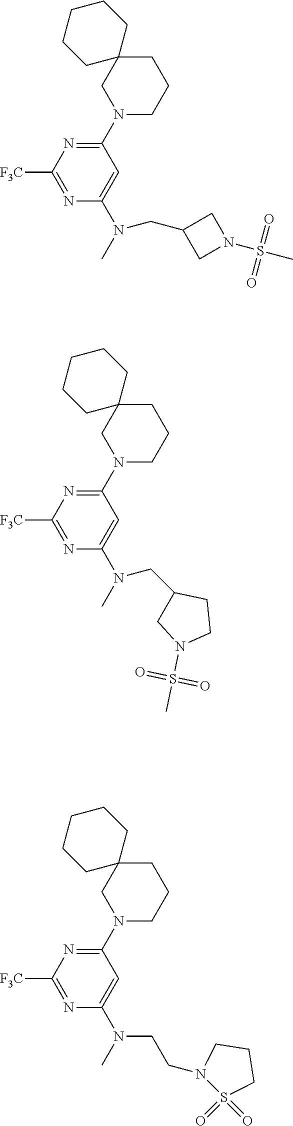 [SMILES: CN(CC1CN(S(C)(=O)=O)C1)c1cc(N2CCCC3(CCCCC3)C2)nc(C(F)(F)F)n1 ; CN(CC1CCN(S(C)(=O)=O)C1)c1cc(N2CCCC3(CCCCC3)C2)nc(C(F)(F)F)n1 ; CN(CCN1CCCS1(=O)=O)c1cc(N2CCCC3(CCCCC3)C2)nc(C(F)(F)F)n1]

In some embodiments, the compound has a structure according to Formula Ib, wherein X is O and Y is CH₂.

In some embodiments, the compound having a structure according to Formula Ib has a structure selected from the group consisting of:

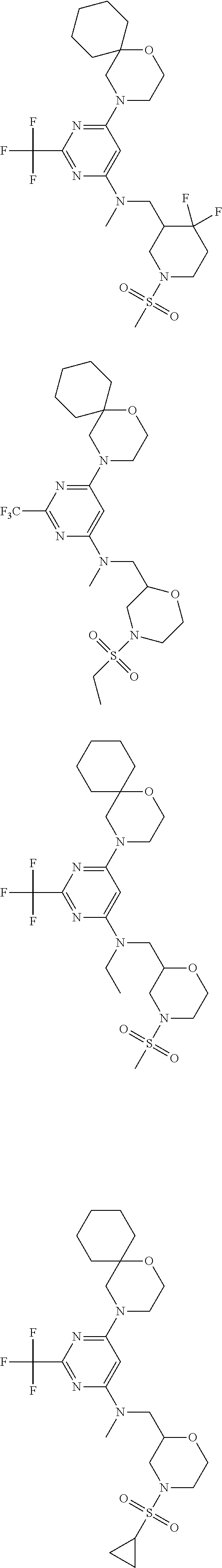

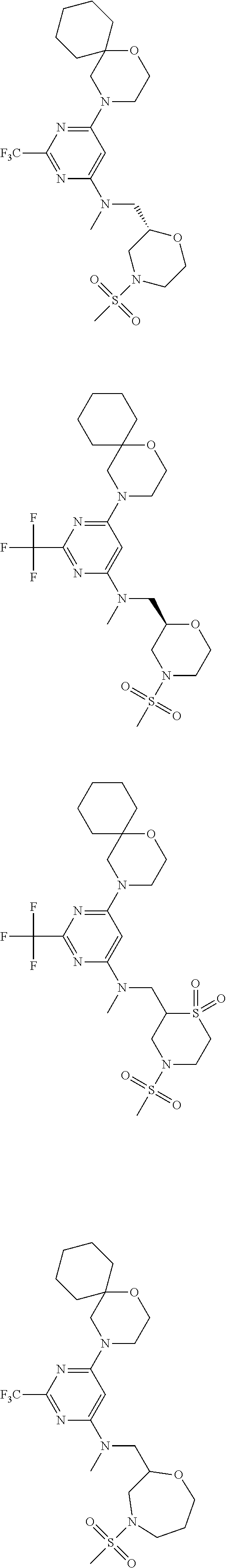 [SMILES: CN(C[C@H]1CN(S(C)(=O)=O)CCO1)c1cc(N2CCOC3(CCCCC3)C2)nc(C(F)(F)F)n1 ; CN(C[C@@H]1CN(S(C)(=O)=O)CCO1)c1cc(N2CCOC3(CCCCC3)C2)nc(C(F)(F)F)n1 ; CN(CC1CN(S(C)(=O)=O)CCS1(=O)=O)c1cc(N2CCOC3(CCCCC3)C2)nc(C(F)(F)F)n1 ; CN(CC1CN(S(C)(=O)=O)CCCO1)c1cc(N2CCOC3(CCCCC3)C2)nc(C(F)(F)F)n1]

In some embodiments, the compound has a structure according to Formula Ib, wherein X is S or SO₂, and Y is CH₂.

In some embodiments, the compound having a structure according to Formula Ib has a structure selected from the group consisting of:

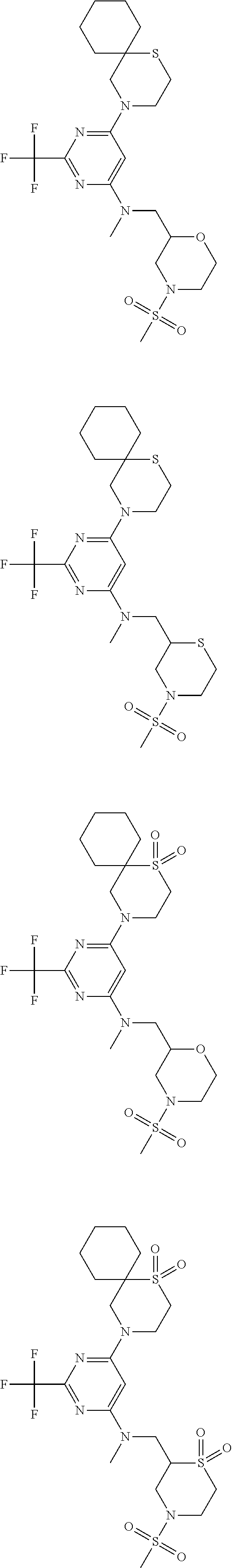 [SMILES: CN(CC1CN(S(C)(=O)=O)CCO1)c1cc(N2CCSC3(CCCCC3)C2)nc(C(F)(F)F)n1 ; CN(CC1CN(S(C)(=O)=O)CCS1)c1cc(N2CCSC3(CCCCC3)C2)nc(C(F)(F)F)n1 ; CN(CC1CN(S(C)(=O)=O)CCO1)c1cc(N2CCS(=O)(=O)C3(CCCCC3)C2)nc(C(F)(F)F)n1 ; CN(CC1CN(S(C)(=O)=O)CCS1(=O)=O)c1cc(N2CCS(=O)(=O)C3(CCCCC3)C2)nc(C(F)(F)F)n1]

In some embodiments, the compound has a structure according to Formula Ib, wherein X is CF₂, and Y is CH₂.

In some embodiments, the compound having a structure according to Formula Ib has a structure selected from the group consisting of.

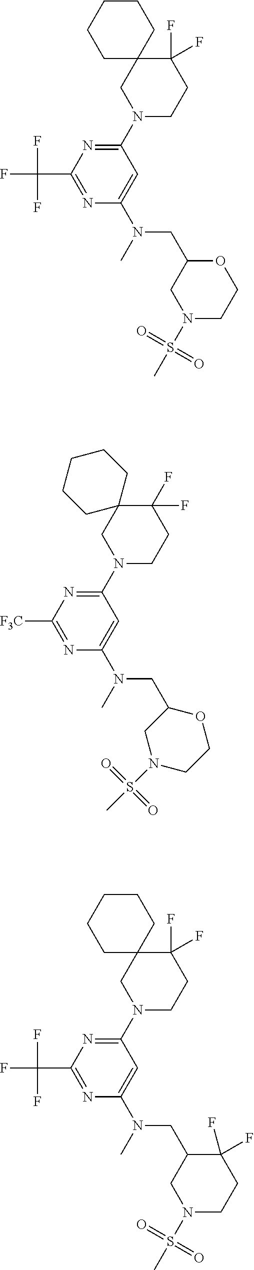 [SMILES: CN(CC1CN(S(C)(=O)=O)CCO1)c1cc(N2CCC(F)(F)C3(CCCCC3)C2)nc(C(F)(F)F)n1 ; CN(CC1CN(S(C)(=O)=O)CCO1)c1cc(N2CCC(F)(F)C3(CCCCC3)C2)nc(C(F)(F)F)n1 ; CN(CC1CN(S(C)(=O)=O)CCC1(F)F)c1cc(N2CCC(F)(F)C3(CCCCC3)C2)nc(C(F)(F)F)n1]

In some embodiments, the compound has a structure according to Formula Ib, wherein X is CH₂, and Y is CF₂.

In some embodiments, the compound has a structure according to Formula Ib, wherein X is O, and Y is a bond.

In some embodiments, the compound has a structure according to Formula I, wherein R₂ and R₃, together with the nitrogen atom to which they are attached, form a 4 to 11 membered bicyclic ring system, the mono- or bicyclic ring system optionally comprising one or more heteroatoms selected from O, N, and S.

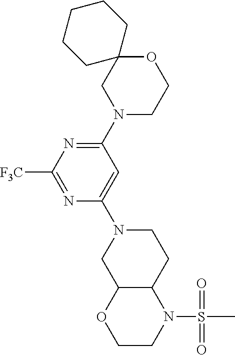 [SMILES: CS(=O)(=O)N1CCOC2CN(c3cc(N4CCOC5(CCCCC5)C4)nc(C(F)(F)F)n3)CCC21]

In some embodiments, the compound has a structure according to Formula I, wherein R₂ and R₃, together with the nitrogen atom to which they are attached, form a 7 to 11 membered spiro-fused ring system, the spiro-fused ring system optionally comprising one or more heteroatoms selected from O, N, and S.

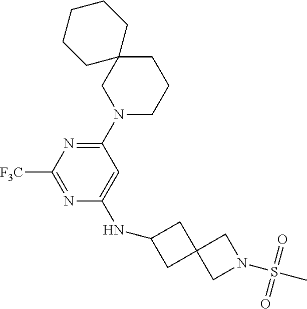 [SMILES: CS(=O)(=O)N1CC2(CC(Nc3cc(N4CCCC5(CCCCC5)C4)nc(C(F)(F)F)n3)C2)C1]

### Isomers

In some embodiments, compounds of the disclosure may be enriched to provide predominantly one enantiomer of a compound described herein. An enantiomerically enriched mixture may comprise, for example, at least 60 mol percent of one enantiomer, or at least 75, at least 80, at least 85, at least 90, at least 95, at least 96, at least 97, at least 98, at least 99, at least 99.5 or even 100 mol percent. In some embodiments, the compounds described herein enriched in one enantiomer may be substantially free of the other enantiomer, wherein substantially free may mean that the other enantiomer makes up less than 10%, or less than 5%, or less than 4%, or less than 3%, or less than 2%, or less than 1% as compared to the amount of the target enantiomer, e.g., in the compound mixture. For example, if a compound mixture contains 98 grams of a first enantiomer and 2 grams of a second enantiomer, it would be said to contain 98 mol percent of the first enantiomer and only 2 mol percent of the second enantiomer.

In some embodiments, the compounds of the disclosure may be enriched to provide predominantly one diastereomer of a compound disclosed herein. A diastereomerically enriched mixture may comprise, for example, at least 60 mol percent of one diastereomer, or at least 75, at least 80, at least 85, at least 90, at least 95, at least 96, at least 97, at least 98, at least 99, at least 99.5, or even 100 mol percent. In some embodiments, the compounds described herein enriched in one diastereomer may be substantially free of other diastereomers, wherein substantially free may mean that the other diastereomers make up less than 10%, or less than 5%, or less than 4%, or less than 3%, or less than 2%, or less than 1% as compared to the amount of the target diastereomer, e.g., in the compound mixture.

Diastereomeric mixtures can be separated into their individual diastereomers on the basis of their physical chemical differences by methods well known to those skilled in the art, such as, for example, by chromatography and/or fractional crystallization. Enantiomers may be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column. Also, some of the compounds of the disclosure may be atropisomers or rotameric forms and are considered as part of this disclosure.

### Isotopes and Isotopically Labeled Compounds

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. Accordingly, reference to a certain element is meant to include all isotopes of that element. For example, if an R group is defined to include hydrogen or H, it also includes isotopes thereof. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically enriched compounds may be prepared by conventional techniques well known to those skilled in the art.

The compounds described herein further include all pharmaceutically acceptable isotopically labeled compounds. An “isotopically” or “radio-labeled” compound may be a compound where one or more atoms are replaced or substituted by an atom having an atomic mass or mass number different from the atomic mass or mass number typically found in nature (i.e., naturally occurring). For example, in some embodiments, in the compounds described herein hydrogen atoms are replaced or substituted by one or more deuterium or tritium.

Certain isotopically labeled compounds of this disclosure, for example, those incorporating a radioactive isotope, may be useful in drug and/or substrate tissue distribution studies. The radioactive isotopes tritium, i.e., ³H, and carbon 14, i.e., ¹⁴C, may be particularly useful for this purpose in view of their ease of incorporation and ready means of detection. Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances. In some embodiments, the compound comprises at least one deuterium atom. For example, one or more hydrogen atoms in a compound of the present disclosure can be replaced or substituted by deuterium. In some embodiments, the compound comprises two or more deuterium atoms. In some embodiments, the compound comprises 1, 2, 3, 4, 5, 6, 7, 8, 9, 10, 11 or 12 deuterium atoms. Suitable isotopes that may be incorporated in compounds described herein include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I. Substitution with positron emitting isotopes, such as ¹¹C, ¹⁸F, ¹⁵O, and ¹³N, can be useful in Positron Emission Topography (PET) studies.

Isotopically labelled versions of the compounds disclosed herein can generally be prepared by following procedures analogous to those disclosed in the Schemes and/or in the Examples herein, by substituting an appropriate isotopically labelled reagent for a non-isotopically labelled reagent.

### Metabolites

The disclosure herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure may include compounds produced by a process comprising administering a compound of this disclosure to a subject, e.g., a mammal, for a period of time sufficient to yield a metabolic product thereof. Such products are typically identified by administering a radiolabeled compound of the disclosure in a detectable dose to subject, such as rat, mouse, guinea pig, monkey, or to human, allowing sufficient time for metabolism to occur, and isolating its conversion products from the urine, blood or other biological samples.

### Salts and Solvates

The present disclosure further provides pharmaceutically or veterinarily acceptable salts, solvates (e.g., hydrates), and combinations thereof of any of the compounds as disclosed herein. The use of the terms “salt,” “hydrate,” “solvate,” and the like, is intended to equally apply to the salt, hydrate, or solvate of enantiomers, diastereomers, isomers, stereoisomers, rotamers, tautomers, positional isomers, or racemates of the disclosed compounds.

The term “salt” or “pharmaceutically acceptable salt” or “veterinarily acceptable salt” refers to salts derived from a variety of organic and inorganic counter ions well known in the art. Pharmaceutically or veterinarily acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically or veterinarily acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the base addition salt is chosen from ammonium, potassium, sodium, calcium, and magnesium salts. In some embodiments, the pharmaceutically or veterinarily acceptable salts may include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, 1,1-methene-bis-2-hydroxy-3-naphthoate, einbonate, pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

Suitable anionic salt forms include, but are not limited to acetate, benzenesulfonate, benzoate, benzylate, bicarbonate, bitartrate, bitartrate, bromide, calcium edetate, camsylateh, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionatei, lactate, lactobionate, malate, maleate, mandelate, mesylate, methylbromide, methylnitrate, methylsulfate, mucate, napsylate, nitrate, pamoate (embonate), pantothenate, phosphate and diphosphate, polygalacturonate, salicylate and disalicylate, stearate, subacetate, succinate, sulfate, tannate, tartrate, teoclate, tosylate, triethiodide, valerate, and the like.

Suitable cationic salt forms include, but are not limited to aluminum, benzathine, calcium, ethylene diamine, lysine, magnesium, meglumine, potassium, procaine, sodium, tromethamine, zinc, and the like. Suitable cationic salt forms include, but are not limited to benzathine, chloroprocaine, choline, diethanolamine, ethylenediamine, meglumine, procaine, aluminum, calcium, lithium, magnesium, potassium, sodium, zinc, and the like.

In some embodiments, the salt is selected from the group consisting of acetate, ascorbate, aspartate, benzoate, besylate, bicarbonate/carbonate, bisulfate/sulfate, borate, camsylate, citrate, edisylate, etoglutarate, esylate, formate, fumarate, gluceptate, gluconate, glucuronate, glycerophosphate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, and trifluoroacetate.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds such as spray dried dispersions, as well as mixtures thereof.

Compounds of the disclosure, including their stereoisomers and tautomers, as well as salts of any thereof, may exist as solvates. Often, crystallizations produce a solvate of the compound of the disclosure. As used herein, the term “solvate” may refer to an aggregate that comprises one or more molecules of a compound of the disclosure with one or more molecules of solvent. The solvent may be water, in which case the solvate may be a hydrate. Alternatively, the solvent may be an organic solvent. Thus, the compounds and salts of the present disclosure may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases the compound of the disclosure may merely retain adventitious water or be a mixture of water plus some adventitious solvent.

### Preparation of Compounds of the Disclosure

Compounds of the present disclosure may be prepared by methods known in the art of organic synthesis as set forth in part by the synthetic schemes in the following description and examples in conjunction with the guidance provided herein. In the schemes described below and provided in the Figures, it is understood that protecting groups for sensitive or reactive groups may be employed where necessary in accordance with general principles or chemistry in accordance with the guidance provided herein. Protecting groups may be manipulated according to standard methods of organic synthesis (T. W. Greene and P. G. M. Wuts, “Protective Groups in Organic Synthesis,” Third edition, Wiley, New York 1999). These groups may be removed at a convenient stage of the compound synthesis using methods that are readily apparent to those skilled in the art based on the detailed teaching provided herein. The selection processes, as well as the reaction conditions and order of their execution, shall be consistent with the present disclosure.

Generally, the methods of preparing compounds of the present disclosure comprise combinations of reactions and conditions. By way of example, and not limitation, certain compounds can be prepared as outlined in the schemes shown below as well as in the examples set forth herein. It should be noted that one skilled in the art will understand how to modify the procedures set forth in the schemes and examples to arrive at the desired products.

General Schemes 1 and 2 illustrate representative, non-limiting strategies for preparation of compounds of the disclosure according to Formula I.

### Scheme 1. Preparation of Compounds of Formula I—Option 1

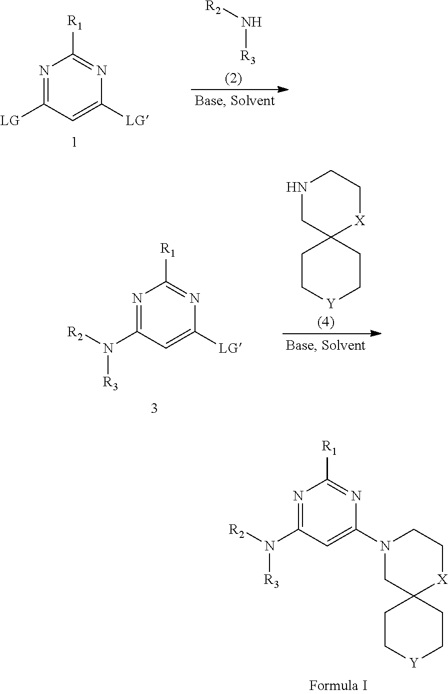

In some embodiments, the compound of Formula I is prepared according to general Scheme 1. With reference to Scheme 1, a pyrimidine 1 bearing substituents LG and LG′ at the 4 and 6 position, respectively, serves as the starting material. The substituents LG and LG′ represent leaving groups, which may be the same or different. Suitable leaving groups include, but are not limited to, chloro or bromo. Substituent R₁ is as defined above with respect to Formula I. In some embodiments, R₁ is H, CH₃, or CF₃. With continued reference to Scheme 1, the pyrimidine 1 is allowed to react with a suitable primary or secondary amine 2 having a formula R₂R₃NH, where R₂ and R₃ are as defined above with respect to Formula I, in a solvent and in the presence of a base.

The amine component 2 is generally either commercially available or readily prepared according to methods known in the art and/or as described herein in the Examples.

Suitable solvents are generally polar and aprotic in nature. Suitable solvents include, but are not limited to, dimethyl formamide, dimethylacetamide, N-methylpyrrolidone, dimethyl sulfoxide, and the like. In some embodiments, the solvent is dimethyl formamide or N-methyl pyrrolidone.

Suitable bases include but are not limited to tertiary amines. Suitable tertiary amine bases include, but are not limited to triethylamine and diisopropylethylamine. In some embodiments, the base is triethylamine, diisopropylethylamine, or a combination thereof.

With continued reference to Scheme 1, the reaction may be conducted at a range of temperatures, and for a period of time suitable to convert the majority of pyrimidine 1 to pyrimidine intermediate 3. In some embodiments, the reaction is performed at a temperature in a range from about 0 °C to about 200 °C, such as from about 0 °C to about 100 °C, about 0 °C to about 25 °C, or from about 20 °C to about 80 °C.

With continued reference to Scheme I, pyrimidine intermediate 3 is allowed to react with a cyclic amine 4, where X and Y as defined above with respect to Formula I, in a solvent and in the presence of a base. One of skill in the art will recognize that alternatively, in structure 2, 3, and 4 of Scheme I, groups X and Y may represent protected versions of or precursors of the actual groups X and Y described with respect to Formula I. Such precursors and protecting groups, and deprotection/conversion of same to the compound of Formula I, will be known to one of skill in the art.

The amine component 4 is generally either commercially available or readily prepared according to methods known in the art and/or as described herein in the Examples.

Suitable solvents are generally polar and aprotic in nature. Suitable solvents include, but are not limited to, dimethyl formamide, dimethylacetamide, N-methylpyrrolidone, dimethyl sulfoxide, and the like. In some embodiments, the solvent is dimethyl formamide or N-methyl pyrrolidone.

Suitable bases include but are not limited to tertiary amines. Suitable tertiary amine bases include, but are not limited to triethylamine and diisopropylethylamine. In some embodiments, the base is triethylamine, diisopropylethylamine, or a combination thereof.

In some embodiments, the solvent and base are the same for each N-alkylation step.

With continued reference to Scheme I, the reaction may be conducted at a range of temperatures, and for a period of time suitable to convert the majority of pyrimidine intermediate 3 to the final product (i.e., a compound of Formula I). In some embodiments, the reaction is performed at a temperature in a range from about 0 °C to about 200 °C, such as from about 50 °C to about 180 °C, or from about 80 °C to about 160 °C. Generally, a higher temperature is utilized for the second N-alkylation reaction relative to the first N-alkylation reaction.

Scheme 2. Preparation of compounds of Formula I—Option 2

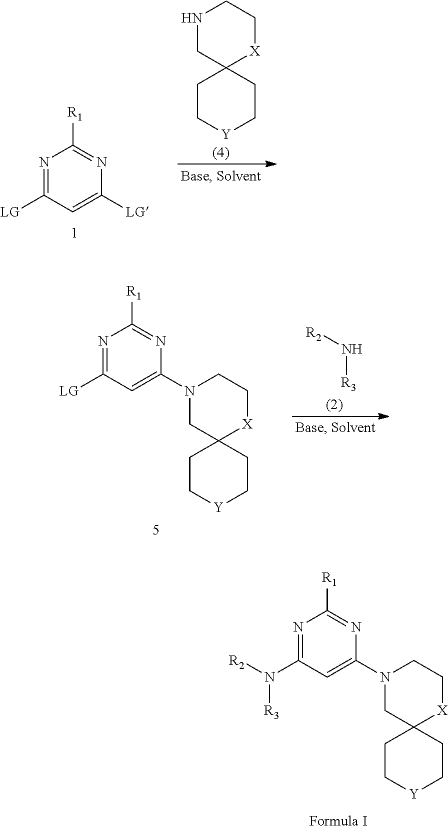

In some embodiments, the compound of Formula I is prepared according to general Scheme 2. Each of the reactants (1, 2, and 4) are the same as in Scheme 1, but the order of addition of reaction of the two amines (2 and 4) differ relative to Scheme 1, and the intermediate product 5 differs in structure from intermediate 3 of Scheme 1. One of skill in the art will recognize that the bases, solvents, and reaction conditions described with respect to Scheme 1 are also suitable for use according to Scheme 2.

The reactions and conditions described above with respect to Schemes 1 and 2 are not intended to be limiting, and alternative routes for the preparation of compounds of Formula I are contemplated herein, or may be known to one of skill in the art.

### Compositions

Although the compounds of Formula I provided herein can be used (e.g., administered) alone, the compounds are generally more advantageously provided in a composition for use in animal health, human health, or agriculture. Typically, such compositions comprise a therapeutically or agriculturally effective amount of the compound(s) and at least one carrier, e.g., a veterinarily acceptable carrier, pharmaceutically acceptable carrier, and/or agriculturally acceptable carrier. The term “carrier” is used herein to describe any component (e.g., excipient, diluent, and the like) other than the compound of the present disclosure or any additional optional active agent incorporated within the composition (e.g., a further antiparasitic agent, such as those referenced herein below). Compositions of the disclosure can be prepared, for example, by combining one or more compounds of the disclosure (or one or more salts, solvates, stereoisomers, or tautomers thereof), with an appropriate carrier, and may be formulated into preparations in solid, semi solid, liquid, or gaseous form.

A compound of the present disclosure, as well as its salts, solvates, isomers, and the like, can be administered to the subject by itself or in a composition where it is mixed with one or more biologically suitable and pharmaceutically/veterinarily acceptable carriers or excipients. The compositions can be in various forms, including, but not limited to, oral formulations, injectable formulations, suppository formulations, and topical, dermal, or subdermal formulations. Selection of an appropriate formulation of a compound as provided herein may be based, e.g., on the physicochemical properties of the compound (and any other optional active agent to be administered), the type of animal being treated, the condition of the animal being treated, and cost.

Compositions can comprise at least the compounds or salts described herein and one or more carriers, diluents, excipients, stabilizers, dispersing agents, suspending agents, and/or thickening agents. The particular components and the relative amounts of each will vary depending on, for example, the intended route of administration. The compositions can be formulated to contain a single daily dose or a convenient fraction of a daily dose in a dosage unit (e.g., a single tablet, single capsule, convenient volume of liquid/ointment, etc.). The amount of the compound of the formula provided above included within a given composition for use in animal and human health can vary widely. The compound is typically included within a composition in an amount likely to induce the desired effect. Compositions that will be administered to a subject or patient commonly take the form of one or more dosage units, where for example, a tablet may be a single dosage unit, and a container of one or more compounds of the disclosure, or acceptable salts, solvates, stereoisomers, or tautomers thereof, in aerosol form may hold a plurality of dosage units.

Compositions for use in accordance with the present disclosure may be formulated in a conventional manner using one or more acceptable carriers comprising excipients and auxiliaries, which facilitate processing of the active compound into preparations, which can be used pharmaceutically or veterinarily. Proper formulation is dependent upon the route of administration chosen. Acceptable excipients and carriers are generally known to those skilled in the art and are thus included in the instant disclosure. Such excipients and carriers are described, for example, in “Remingtons Pharmaceutical Sciences” Mack Pub. Co., New Jersey (1991). In general, compositions of the disclosed compounds may be manufactured in a manner that is itself known, e.g., by means of conventional mixing, dissolving, granulating, dragee-making, levigating, emulsifying, encapsulating, entrapping or lyophilizing processes. The techniques for formulation may be found in references well known to one of ordinary skill in the art, such as “Remington's Pharmaceutical Sciences,” Mack Publishing Co., Easton, PA, latest edition.

### Oral Dose Forms

The compositions described herein can be formulated for oral administration. When intended for oral administration, pharmaceutical compositions of the present disclosure typically are either solid or liquid form, where semi solid, semi liquid, suspension and gel forms may be included within the forms considered herein as either solid or liquid.

Solid dosage forms for oral administration include capsules, tablets, pills, powders, chews (e.g., flavored treats) and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, i) lubricants such as talc, calcium stearate, magnesium stearate, solid polyethylene glycols, sodium lauryl sulfate, and mixtures thereof, and j) flavoring agents. In the case of capsules, tablets and pills, the dosage form may also comprise buffering agents. When the pharmaceutical composition is in the form of a capsule, for example, a gelatin capsule, it may contain, in addition to materials disclosed herein, a liquid carrier such as polyethylene glycol or oil.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. In some embodiments, a capsule (a hard capsule or a soft capsule), e.g., a gelatin capsule comprising a compound according to a formula as described herein is provided. A hard gelatin capsule, in some embodiments, can a compound according to a formula as described herein admixed with an inert solid diluent, e.g., a starch, powdered cellulose (e.g., crystalline or microcrystalline cellulose), a sugar (e.g., fructose, mannitol, or sucrose), a grain flour, calcium carbonate, calcium phosphate, and/or kaolin, enclosed in a capsule. A soft or liquid gelatin capsule, in some embodiments, can comprise a compound according to a formula as described herein mixed with water or a solvent such as propylene glycol, polyethylene glycol (PEG), and/or ethanol or mixed with an oil medium, e.g., peanut oil, liquid paraffin, or olive oil, enclosed within a capsule. In some embodiments, such capsules are microcapsules.

In some embodiments, a composition in tablet form is provided, comprising a compound according to a formula as described herein in combination with one or more pharmaceutically acceptable excipients. Tablets are generally prepared via direct compression (e.g., wet granulation or dry granulation of ingredients). Excipients known to be suitable for the manufacture of tablets are generally known and include, for example, inert diluents (e.g., starches, lactose, mannitol, powdered sugar, powdered cellulose derivatives, kaolin, calcium carbonate, sodium carbonate, lactose, calcium phosphate, calcium sulfate, sodium phosphate, and/or inorganic salts such as sodium chloride); granulating and disintegrating agents (e.g., starch such as corn or potato starch, clay, cellulose, methylcellulose, carboxymethyl cellulose, algins, and/or alginic acid, agar, bentonite, wood cellulose, powdered natural sponge, cation-exchange resins, guar gum, citrus pulp, and/or sodium lauryl sulfate); binding agents (e.g., starch, gelatin, sugars (e.g., lactose, fructose, glucose, and the like), natural or synthetic gums such as acacia, alginates, methylcellulose, polyvinylpyrrolidine, and the like), polyethylene glycol, ethyl cellulose, and/or waxes; and lubricating agents, (e.g., magnesium stearate, calcium stearate, stearic acid, hydrogenated vegetable oil, and/or talc). Tablets may be uncoated or may be coated by known techniques (which can serve to delay disintegration and absorption in the gastrointestinal tract and thereby provide for sustained action over a longer period of time). For example, in some embodiments, a time delay material such as glyceryl monostearate or glyceryl distearate may be employed. Tablets may also be optionally coated by techniques described in U.S. Pat. No. 4,256,108 to Theeuwes; U.S. Pat. No. 4,166,452 to Generales Jr.; and U.S. Pat. No. 4,265,874 to Bonsen et al., which are all incorporated herein by reference with respect to the preparation of osmotic therapeutic tablets for controlled release. Troches are an example of tablets, and are typically formulated as small, hard tablets which dissolve slowly when placed under the tongue. Tablets can optionally be coated, e.g., with sugar as a flavorant and sealant or with film-forming protecting agents to modify the dissolution properties of the tablet.

Compositions of the disclosure may be in the form of a liquid, for example, an elixir, syrup, solution, emulsion or suspension. The liquid may be for oral administration or for delivery by injection, as two examples. When intended for oral administration, compositions of the disclosure typically contain, in addition to one or more compounds of the disclosure, or acceptable salts, solvates, stereoisomers, or tautomers thereof, one or more of a sweetening agent, preservatives, dye/colorant and flavor enhancer.

In some embodiments, a composition comprising a compound according to Formula I as described herein is provided in the form of an emulsion, e.g., in the form of an oil-in-water or a water-in-oil emulsion. The oily phase of the emulsion may be a vegetable oil, e.g., olive oil or arachis oil or may be a mineral oil, e.g., liquid paraffin, or a mixture of any such oils. In some embodiments, the oily phase is formed from unsaturated polyglycosylated glycerides, triglycerides, (e.g., medium-chain triglycerides, such as C₈-C₁₀ caprylic/capric triglycerides), or combinations thereof. The aqueous phase can comprise water or glycol derivatives (e.g., propylene glycol, glycol ether, polyethylene glycol, or glycerol). Specific examples include, but are not limited to, propylene glycol, diethylene glycol monoethyl ether, dipropylene glycol monomethyl ether, and mixtures thereof. Suitable emulsifying agents may include, e.g., naturally occurring phosphatides, e.g., soybean, lecithin, and esters or partial esters derived from fatty acids and hexitol anhydrides, e.g., sorbitan monoleate, and condensation products of the referenced partial esters with ethylene oxide, e.g., polyoxyethylene sorbitan monooleate. The emulsions may also optionally contain sweetening agents, bittering agents, flavoring agents, and/or preservatives. In one embodiment, the emulsion is in the form of a microemulsion (composed of stable dispersions of microdroplets of the aqueous phase in the oily phase or of microdroplets of the oily phase in the aqueous phase). Microemulsions are quaternary systems comprising an aqueous phase, an oily phase, a surfactant and a co-surfactant. They are translucent and isotropic liquids. The size of these microdroplets is less than 200 nm (in contrast to microdroplets with sizes of about 1000 nm to 100,000 nm for emulsions). In some embodiments, the oily phase will represent a % v/v range selected from the group consisting of about 2 to about 15%; about 7 to about 10%; and about 8 to about 9% v/v of the microemulsion. Generally, the aqueous phase will represent a proportion from about 1 to about 4% v/v in the microemulsion. The interfacial film is composed of an alternation of surface-active (SA) and co-surface-active (Co-SA) molecules which, by lowering the interfacial tension, allows the microemulsion to be formed spontaneously. Surfactants for the microemulsion include diethylene glycol monoethyl ether, dipropylene glycol monomethyl ether, polyglycolyzed C₈-C₁₀ glycerides or polyglyceryl-6 dioleate. In addition to these surfactants, the co-surfactants include short-chain alcohols, such as ethanol and propanol. Some compounds are common to the three components discussed above, i.e., aqueous phase, surfactant and co-surfactant. However, it is well within the skill level of the practitioner to use different compounds for each component of the same formulation. In one embodiment, the co-surfactant to surfactant ratio will be from about 1/7 to about 1/2. In another embodiment, there will be from about 25 to about 75% v/v of surfactant and from about 10 to about 55% v/v of co-surfactant in the microemulsion.

In some embodiments, a composition comprising a compound according to Formula I as described herein is provided in the form of a suspension, generally comprising the compound (optionally along with other ingredients) dispersed in a liquid. The compound is typically in the form of a dispersible powder or granule; dispersible powders and granules suitable for preparation of a suspension generally provide the active ingredient in admixture with a dispersing or wetting agent, suspending agent and one or more preservatives. Suitable dispersing or wetting agents and suspending agents are exemplified by those already mentioned above. Additional excipients, for example, sweetening, bittering, flavoring, and coloring agents, may also be present. The liquid can be oily or aqueous. Oily suspensions may be formulated by suspending the compound in a vegetable oil, for example, atachis oil, olive oil, sesame oil or coconut oil, or in mineral oil such as liquid paraffin. The oily suspensions may contain a thickening agent, for example, beeswax, hard paraffin or cetyl alcohol. Sweetening agents such as sucrose, saccharin or aspartame, bittering agents, and flavoring agents may be added to provide a palatable oral preparation. These compositions may be preserved by the addition of an antioxidant such as ascorbic acid, or other known preservatives. Aqueous suspensions contain the compound in admixture with excipients suitable for the manufacture of aqueous suspensions. For example, such aqueous suspensions may comprise excipients that are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide, with partial esters derived from fatty acids and hexitol anhydrides, for example polyethylene sorbitan monooleate. Aqueous suspensions may also contain one or more preservatives, for example ethyl, or n-propyl, p-hydroxybenzoate, one or more coloring agents, one or more flavoring agents, and one or more sweetening agents and/or bittering agents, such as those set forth above. Aqueous suspensions may comprise, e.g., juice (such as apple or orange juice).

In some embodiments, a composition comprising a compound according to Formula I as described herein is provided in the form of a syrup or elixir. Syrups and elixirs may be formulated with sweetening agents, for example, glycerol, propylene glycol, sorbitol, or sucrose. Such formulations may also contain a demulcent, a preservative, flavoring agent(s) and/or coloring agent(s).

In some embodiments, a composition comprising a compound according to Formula I as described herein is in paste form. Examples of embodiments in a paste form include but are not limited to those described in U.S. Pat. No. 6,787,342 to Chen; U.S. Pat. No. 7,001,889 to Freehauf et al.; and U.S. Pat. No. 7,563,773 to Freehauf, each of which is incorporated herein by reference in its entirety. In addition to the compound(s) of the disclosure, the paste can also contain components including, e.g., fumed silica; a viscosity modifier (e.g., selected from PEG 200, PEG 300, PEG 400, PEG 600, monoethanolamine, triethanolamine, glycerol, propylene glycol, polyoxyethylene (20) sorbitan monooleate (polysorbate 80 or Tween 80), and polyoxamers (e.g., Pluronic L 81)); a carrier (e.g., a hydrophilic carrier selected from triacetin, a monoglyceride, a diglyceride, and a triglyceride); optionally, an absorbent (e.g., selected from magnesium carbonate, calcium carbonate, starch, and cellulose and its derivatives); and optionally, a colorant (e.g., selected from the group consisting of titanium dioxide iron oxide, and FD&C Blue #1 Aluminum Lake), stabilizer, surfactant, and/or preservative.

Sustained-release preparations can also be prepared. Examples of sustained-release preparations can include semipermeable matrices of solid hydrophobic polymers that can contain the compound or salt, and these matrices can be in the form of shaped articles (e.g., films or microcapsules). Examples of sustained-release matrices can include polyesters, hydrogels (e.g., poly(2-hydroxyethyl-methacrylate), or poly (vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

### Injectable Forms

The compositions described herein can be formulated for administration as an injection. In a composition intended to be administered by injection, one or more of a surfactant, preservative, wetting agent, dispersing agent, suspending agent, buffer, stabilizer and isotonic agent may be included. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions described herein can be lyophilized or in powder form for reconstitution with a suitable vehicle, e.g., sterile pyrogen-free water, before use.

For parenteral administration, the compounds or salts can be formulated in a unit dosage injectable form (e.g., use letter solution, suspension, emulsion) in association with a pharmaceutically acceptable parenteral vehicle. Such vehicles can be inherently non-toxic, and non-therapeutic. Vehicles can be water, saline, Ringer's solution, dextrose solution, and 5% human serum albumin. Non-aqueous vehicles such as triacetin, butyldigol, miglyol, N-methylpyrollidinone, benzyl benzoate, glycerol formal, dipropylene glycol monomethyl ether, tetraglycol, fixed oils and ethyl oleate can also be used. Liposomes can be used as carriers. The vehicle can contain minor amounts of additives such as substances that enhance isotonicity and chemical stability (e.g., buffers and preservatives).

Liquid compositions of the disclosure, whether they be solutions, suspensions or other like form, may include one or more of the following adjuvants: sterile diluents such as water for injection, saline solution, physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. Parenteral preparations can be enclosed in ampoules, disposable syringes or multiple dose vials made of glass or plastic. In some embodiments, the adjuvant is physiological saline. In some embodiments, the injectable pharmaceutical composition is sterile.

### Other Dosage Forms

Compositions of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration.

In some embodiments, a composition suitable for topical, dermal, and/or subdermal formulation comprising a compound according to Formula I as described herein is provided. Topical, dermal, and subdermal formulations can include, e.g., emulsions, creams, ointments, gels, pastes, powders, patches, shampoos, pour-on formulations, ready-to-use formulations, spray formulations, and spot-on formulations. Such formulations can be, e.g., concentrated solutions, suspensions, microemulsions, or emulsions. Topical application can, in some embodiments, allow for the active compound(s) to be distributed through the glands (e.g., sebaceous glands) of the animal and/or allow the active compound(s) to achieve a systemic effect (plasma concentration) and/or allow for distribution throughout the haircoat. Certain suitable formulations for topical, dermal, and/or subdermal application include, but are not limited to, those disclosed in U.S. Pat. No. 6,395,765 to Etchegaray, which is incorporated herein by reference in its entirety.

Spot-on compositions are typically applied in a localized region which refers to an area other than the entire animal. Spot-on compositions are generally used by administering the composition at a particular location of an animal (e.g., between the shoulders). Another embodiment of a localized region is a stripe, e.g., a stripe from head to tail of the animal. Such compositions can be administered, e.g., via pipettes, squeeze-ons, or drop-ons.

The carrier can be a liquid carrier vehicle as described in U.S. Pat. No. 6,426,333, which is incorporated herein by reference in its entirety. For example, in one embodiment, a spot-on formulation comprises a solvent and a co-solvent wherein the solvent is selected from the group consisting of acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol, diethyl phthalate fatty acid esters, such as the diethyl ester or diisobutyl adipate, and a mixture of at least two of these solvents and the co-solvent is selected from the group consisting of absolute ethanol, isopropanol or methanol. The liquid carrier vehicle can optionally contain a crystallization inhibitor selected from the group consisting of an anionic surfactant, a cationic surfactant, a non-ionic surfactant, an amine salt, an amphoteric surfactant or polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose, and acrylic derivatives, or a mixture of these crystallization inhibitors.

Pour-on compositions are generally used by pouring the composition along an animal's backline (e.g., from the neck to the tail). The pour-on formulations are advantageously oily, and generally comprise a diluent or vehicle and also a solvent (e.g., an organic solvent) for the active ingredient if the latter is not soluble in the diluent. Certain non-limiting pour-on compositions are disclosed, for example, in U.S. Pat. No. 6,010,710 to Etchegaray and U.S. Pat. No. 8,097,266 to Gogolewski et al., which are incorporated herein by reference in their entireties.

Spray-on compositions are generally used by spraying a composition along an animal's backline (e.g., from the neck to the tail). Each of these compositions can involve application, e.g., of a concentrated solution, suspension, microemulsion or emulsion.

Such topical compositions (e.g., spot-on, spray-on, and pour-on compositions) can generally comprise the compound as provided herein in combination with one or more diluents/vehicles and/or one or more solvents. Diluents/vehicles include, but are not limited to, plant oils (e.g., soybean oil, groundnut oil, castor oil, corn oil, cotton oil, olive oil, grape seed oil, sunflower oil, etc.); mineral oils (e.g., petrolatum, paraffin, silicone, etc.); aliphatic or cyclic hydrocarbons; medium-chain (such as C8 to C12) triglycerides, and combinations thereof. Solvents (e.g., organic solvents) that can be added in some embodiments include, but are not limited to, acetyltributyl citrate, fatty acid esters such as the dimethyl ester, diisobutyl adipate, acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol and diethyl phthalate, and mixtures of two or more thereof. In some embodiments, an emollient and/or spreading and/or film-forming agent is included in the composition. An emollient and/or spreading and/or film-forming agent can be, for example, selected from the group consisting of: polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose, silicone oils, polydiorganosiloxane oils (such as polydimethylsiloxane (PDMS) oils), for example those containing silanol functionalities, or a 45V2 oil, anionic surfactants such as alkaline stearates, sodium, potassium or ammonium stearates; calcium stearate, triethanolamine stearate; sodium abietate; alkyl sulfates (e.g. sodium lauryl sulfate and sodium cetyl sulfate); sodium dodecylbenzenesulfonate, sodium dioctylsulphosuccinate; fatty acids (e.g. those derived from coconut oil), cationic surfactants such as water-soluble quaternary ammonium salts of formula N+R′R″R′″R″″, Y− in which the radicals R are optionally hydroxylated hydrocarbon radicals and Y− is an anion of a strong acid such as the halide, sulfate and sulfonate anions; cetyltrimethylammonium bromide is among the cationic surfactants which can be used, amine salts of formula N+R′R″R′″ in which the radicals R are optionally hydroxylated hydrocarbon radicals (e.g., octadecylamine hydrochloride), nonionic surfactants such as sorbitan esters, which are optionally polyoxyethylenated (e.g. polysorbate 80), polyoxyethylenated alkyl ethers; polyoxypropylated fatty alcohols such as polyoxypropylene-styrol ether; polyethylene glycol stearate, polyoxyethylenated derivatives of castor oil, polyglycerol esters, polyoxyethylenated fatty alcohols, polyoxyethylenated fatty acids, copolymers of ethylene oxide and propylene oxide, amphoteric surfactants such as the substituted lauryl compounds of betaine; and mixtures of at least two of these agents.

Compositions of the disclosure may be intended for rectal administration, in the form, for example, of a suppository, which will melt in the rectum and release the drug. Compositions for rectal administration may contain an oleaginous base as a suitable nonirritating excipient. Such bases may include, without limitation, lanolin, cocoa butter and polyethylene glycol.

Compositions of the disclosure may include various materials, which modify the physical form of a solid or liquid dosage unit. For example, the compositions may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

### Activity of Compounds

The compounds and compositions provided herein may be active against one or a number of different parasites. For example, in some embodiments, certain compounds and compositions comprising such compounds are effective against a single species or genus of parasite. In some embodiments, certain compounds and compositions comprising such compounds are effective against a broad spectrum of parasites.

In some embodiments, compounds of the disclosure exhibit anthelmintic activity. In some embodiments, compounds of the disclosure exhibit selective anthelmintic activity at the larval stage of the parasite. For example, in some embodiments, compounds of the disclosure exhibit anthelmintic activity against L1 staged Dirofilaria immitis (heartworm). In some embodiments, compounds of the disclosure have activity against the L3/L4 stage of Dirofilaria immitis.

As described herein below, the activity (i.e., efficacy) against parasites, including but not limited to heartworm, may vary. In some embodiments, compounds of the disclosure may be described as having a degree of efficacy at a particular time point following administration, or an efficacy lasting for a period of time following administration, and/or an efficacy at a time or period of time and specific to a particular lifecycle stage of a parasite. In some embodiments, efficacy is measured in terms of a minimum effective concentration at which larval motility is eliminated. Assays to determine efficacy against parasites (e.g., against heartworm) are known in the art and include but are not limited to the in vitro assay described in Example 44 of the disclosure. In some embodiments, compounds of the disclosure have a minimum effective dose (MED) toward elimination of Dirofilaria immitis L1 larvae of less than about 100 mM, or less than about 30 mM, or less than about 10 mM, such as from about 1 mM to about 10 mM, about 10 mM to about 30 mM, or about 30 mM to about 100 mM.

In some embodiments, the antiparasitic activity is effected by inhibition of an enzyme within the parasite. For example, in some embodiments, a compound of the disclosure exhibits antiparasitic activity by virtue of inhibition of a parasite isoform of the Acetyl-CoA carboxylase (ACC) enzyme. ACC is a biotin-dependent enzyme that catalyzes the irreversible carboxylation of acetyl-CoA in the biosynthesis of fatty acids. ACC is a large, multi-domain enzyme in the cytoplasm of most eukaryotes. When the enzyme is active, malonyl-CoA, a building block for new fatty acids, is produced through its two catalytic activities, biotin carboxylase (BC) and carboxyltransferase (CT). Assays to determine inhibitory activity toward ACC are known in the art and include but are not limited to in the vitro assays such as those described in Example 43 of the disclosure. In some embodiments, compounds of the disclosure have an IC50 for ACC inhibition of less than about 500 nM, such as from 1 nM to about 500 nM. In some embodiments, compounds of the disclosure have an IC50 for ACC inhibition in a range from about 500 nM to about 5 mM. In some embodiments, compounds of the disclosure have an IC50 for ACC inhibition of about 5 mM or greater. In some embodiments, compounds of the disclosure exhibit antiparasitic activity in the absence of potent inhibition of ACC. In some embodiments, compounds of the disclosure exhibit antiparasitic activity and potent inhibition of ACC.

Methods of Treating and/or Preventing Parasitic Infection

The disclosure provides methods of preventing, treating, and/or controlling a parasite infestation and/or infection or parasitic disease, e.g., in an animal. The parasitic infestation or infection can be an ectoparasitic infestation or infection or can be an endoparasitic infestation or infection. In some embodiments, the method is a method of treating a parasitic disease (i.e., a disease caused by one or more of the parasites described herein). The methods comprise administering an effective amount of a compound or veterinary/pharmaceutical composition as described herein to the animal. It is noted that pharmaceutical and veterinary compositions are typically provided in dosage units; according to the disclosed methods, one or more such dosage units can be administered to an animal.

### Administration

The route of administration may depend, e.g., on the type and extent of the infection, infestation, or disease being treated, and/or the type of animal. One of skill in the art will be able to, in some embodiments, select a suitable composition as described above for the desired route of administration. As such, the compound(s) or compositions described herein can be administered to the subject enterally (e.g., orally or rectally), topically, or parenterally (e.g., intravenously, intramuscularly, or subcutaneously).

The amount of compound or composition administered to an animal in need of prevention or treatment can vary widely. The amount of compound administered can depend, e.g., on such factors as the efficacy of the compound, the type of animal being treated, the animal's body weight, the animal's age, the desired effect, the type of infestation/infection to be prevented or treated, and the extent of infestation/infection.

Accordingly, the amount of compound or composition administered to a subject in need of treatment can vary widely. The amount of compound administered can depend, e.g., on such factors as the efficacy of the compound, the type of subject being treated, the subject's body weight, the subject's age, the desired effect, and the nature and severity of the disease. It is to be understood that the dosages may vary depending upon the requirements of each subject and the severity of the disorders or diseases being treated. One of skill in the art will be able to discern a specific efficacious dose. Generally, a therapeutically effective amount is the amount of compound or composition sufficient to provide a beneficial effect or to otherwise reduce a detrimental non-beneficial event to the subject to whom the compound or composition is administered. A therapeutically effective dose can be a dose that produces one or more desired or desirable (e.g., beneficial) effects for which it is administered, such administration occurring one or more times over a given period of time. Also, it is to be understood that an initial, higher dosage (i.e., one or more loading doses) may be administered in order to rapidly achieve the desired plasma concentration. On the other hand, the initial dosage may be smaller than the optimum and the daily dosage may be progressively increased during the course of treatment depending on the particular situation (i.e., dose titration).

Administration of the compounds or compositions provided herein may vary in treatment timing. Administration may, for example, be intermittent in time and can be administered daily, weekly, biweekly, monthly, bimonthly, quarterly, or even for longer durations of time. In some embodiments, administration of the desired dose may be presented as a single dose or as divided doses administered at appropriate intervals, for example, as two, three, four or more sub-doses per day. The sub-dose itself may be further divided, e.g., into a number of discrete, loosely spaced administrations, such as multiple oral dose forms. In some embodiments, the administration is daily for a period of time of at least about one week, two weeks, three weeks, a month, or two months, and up to six months, a year, or multiple years, including for the lifetime of the subject. The time period between treatments can depend upon factors such as the parasite(s) being treated, the degree of infestation, the type of animal being treated, and the environment in which the animal resides. One of skill in the art will be able to develop a specific administration protocol for a particular situation.

In some embodiments, a compound or composition as provided herein is administered in a once-daily dose over a period of time ranging from one day to a period of time of days, weeks, months, or years. In some embodiments, a compound or composition as provided herein is administered twice, three, four, or more times daily. In some embodiments, a compound or composition as provided herein is administered as a single dose every month, every three months, every six months, or every year.

It will be recognized by one of skill in the art that the optimal quantity and spacing of individual dosages of a compound as described herein or a composition comprising the compound as described herein will be determined by the nature and extent of the condition being treated, the form, route and site of administration, and the age and condition of the particular subject being treated, and that a physician or veterinarian will ultimately determine appropriate dosages, frequency and treatment duration to be used. The selected dosage may be repeated as often as appropriate. If side effects develop the amount and/or frequency of the dosage can be altered or reduced, in accordance with normal clinical practice. One of skill in the art will be able to develop a specific administration protocol for a particular situation.

### Animal

Animals to which the disclosed compounds and/or compositions are administered according to the disclosed method can be any type of animal, e.g., mammals, birds, and fish. The animals can be at any stage of development, including embryonic and fetal stages. The animals can be of any age; in some embodiments, the methods relate to the treatment of adult animals. In some embodiments, the methods relate to the treatment of juvenile animals. In some embodiments, the methods relate to the treatment of mammals. In some embodiments, the methods relate to treatment of companion animals (e.g., dogs, cats, llamas, and horses). In some embodiments, the methods relate to treatment of livestock (e.g., swine, camel, rabbits, goat, sheep, deer, elk, cattle, and bison). Certain animals that can be effectively treated include, but are not limited to, humans, cattle, bison, swine, sheep, deer, elk, horses, pigs, chicken, and dogs, as well as horses, zebras, goats, llamas, alpacas, camels, yaks, water buffalo, donkeys, mules, fallow deer, reindeer, cats, rabbits, rodents, fur-bearing animals, such as, for example, mink, chinchilla, raccoon, birds, such as, for example, hens, chickens, geese, turkeys, quails, ostriches, and ducks, and fish such as those of the taxonomic classes Chondrichthyes (e.g., sharks and rays) and Osteichthyes (bony fish), including, but not limited to, shark, salmon, trout, whitefish, catfish, tilapia, seabass, tuna, halibut, turbot, flounder, sole, striped bass, eel, yellowtail, grouper, and the like. In some embodiments, the animals treated are humans and in other embodiments, the animals treated are non-human animals. In some embodiments, the animal is a companion animal, for example, a dog or cat. In specific embodiments, the animal is a dog. In some embodiments, the animal is a fish.

### Pests and Parasites

Parasite infestations and/or infections treated according to methods provided herein can be from various types of pests. Animal pests can be, e.g., ectoparasites or endoparasites. In some embodiments, the pests are ectoparasites selected from insects, arachnids, fleas, ticks, mites, mosquitoes, flies, lice, and/or blowfly. In some embodiments, the pests are endoparasites, e.g., selected from helminths (e.g., filarie) including cestodes (tapeworms), nematodes (roundworms), and trematodes (flatworms or flukes). In some embodiments, compounds and compositions provided herein are active against both ectoparasites and endoparasites.

In some embodiments, the pests are ectoparasitic pests from the order of the Anoplura, for example, Haematopinus spp., Linognathus spp., Solenoptes spp., Pediculus spp., Pthirus spp.; from the order of the Mallophaga, for example, Trimenopon spp., Menopon spp., Eomenacanthus spp., Menacanthus spp., Trichodectes spp., Felicola spp., Damalinea spp., Bovicola spp.; from the order of the Diptera, suborder Brachycera, for example, Chrysops spp., Tabanus spp., Musca spp., Hydrotaea spp., Muscina spp., Haematobosca spp., Haematobia spp., Stomoxys spp., Fannia spp., Glossina spp., Lucilia spp., Calliphora spp., Auchmeromyia spp., Cordylobia spp., Cochliomyia spp., Chrysomyia spp., Sarcophaga spp., Wohlfahrtia spp., Gasterophilus spp., Oedemagena spp., Hypoderma spp., Oestrus spp., Rhinoestrus spp., Melophagus spp., Hippobosca spp.; from the order of the Diptera, suborder Nematocera, for example, Culex spp., Aedes spp., Anopheles spp., Culicoides spp., Phlebotomus spp., Simulium spp.; from the order of the Siphonaptera, for example, Ctenocephalides spp., Echidnophaga spp., Ceratophyllus spp., Pulex spp.; from the order of the Metastigmata, for example, Hyalomma spp., Rhipicephalus spp., Boophilus spp., Amblyomma spp., Haemaphysalis spp., Dermacentor spp., Ixodes spp., Argas spp., Otobius spp.; from the order of the Mesostigmata, for example, Dermanyssus spp., Ornithonyssus spp., Pneumonyssus spp.; from the order of the Prostigmata, for example, Cheyletiella spp., Psorergates spp., Myobia spp., Demodex spp., Neotrombicula spp.; from the order of the Astigmata, for example, Acarus spp., Myocoptes spp., Psoroptes spp., Chorioptes spp., Otodectes spp., Sarcoptes spp., Notoedres spp., Knemidocoptes spp., Neoknemidocoptes spp., Cytodites spp., Laminosioptes spp.; and fleas (Siphonaptera, for example, Ctenocephalides spp., Echidnophaga spp., Ceratophyllus spp., Pulex spp.), ticks (Hyalomma spp., Rhipicephalus spp., Boophilus spp., Amblyomma spp., Haemaphysalis spp., Dermacentor spp., Ixodes spp., Argas spp., Ornithodorus spp., and Otobius spp).

In some embodiments, the parasite is an animal parasite (i.e., a parasite causing infection in an animal, such as a mammal). Specific non-limiting animal parasites against which the disclosed compounds and compositions may be effective include, but are not limited to, cat and dog fleas (Ctenocephalides felis, Ctenocephalides spp. and the like), ticks (Rhipicephalus spp., Ixodes spp., Dermacentor spp., Amblyoma spp. and the like), and mites (Dmodex spp., Sarcoptes spp. (e.g., Sarcoptes scabici), Octodectes spp., Psoroptes sp. (e.g., Psoroptes ovis) and the like), lice (Trichodectes spp., Cheyletiella spp., Lignonathus spp. (e.g., Linognathus vitulorum) and the like), mosquitoes (Aedes spp., Culex spp., Anopheles spp., and the like) and flies (Hematobia spp. (e.g., Haematobia irritans (horn fly)), Musca spp. (e.g. Musca domestica), Stomoxys spp. (e.g., Stomoxys calcitrans (stable fly)), Dematobia spp., Cochliomyia spp., and the like). Further non-limiting parasites include parasites of the tick genus Boophilus (e.g., those of the species microplus (cattle tick), decoloratus and annulatus); myiases such as Dermatobia hominis and Cochliomyia hominivorax (greenbottle); sheep myiases such as Lucilia sericata, Lucilia cuprina (blowfly strike); and migrating dipterous larvae.

In some embodiments, the animal pests are endoparasitic pests, e.g., including, but not limited to, Anaplocephala, Ancylostoma (e.g., Ancylostoma duodenale), Anecator, Ascaris, Capillaria, Caenorhabditis, Capillaria, Cooperia, Dipylidium, Dipyllidinum, Dirofilaria (e.g., D. immitis, D. repens, D. ursi, D. tenuis, D. spectans, D. lutrae, and the like), Echinococcus, Enterobius, Fasciola, Haemonchus (e.g., Haemonchus contortus), Oesophagostumum, Ostertagia, Parascaris, Toxocara, Strongyloides, Toxascaris, Trichinella, Trichuris (e.g., Trichuris trichiura), and Trichostrongylus. Further non-limiting examples of pests are filarial nematodes within the Onchocercidae family, including, e.g., Brugia spp., Wucheria spp., Dirofilaria spp., Dipetalonema spp. (e.g., D. reconditum, D. repens, and the like), Necator spp. (e.g., Necator americanus), Onchocerca spp., Elaeophora spp. (e.g., E. bohmi, E. elaphi, E. poeli, E. sagitta, E. schneideri, and the like), Mansonella spp. (e.g., M. ozzardi, M. perstans, and the like), and Loa spp. (e.g., L. loa).

In some embodiments, a compound or composition as provided herein is used to treat or prevent a disease caused by helminthic infection (e.g., a filarial worm infection). In some embodiments, a compound or composition as provided herein is used to treat a disease caused by parasitic worm infection (including, but not limited to, heartworm disease, ascariasis, trichuriasis, schistosomiasis, haemonchosis, onchocerciasis, and lymphatic filariasis). Parasitic disease can be associated with any of the parasites disclosed herein (e.g., a helminth, including nematodes, cestodes, and trematodes; an insect, an arachnid, or an arthropod, including lice, fleas, flies, ticks, etc.). In some embodiments, the parasitic disease is selected from the group consisting of enterobiasis, oxyuriasis, ascariasis, ancylostomiasis, necatoriasis, dracunculiasis, elephantiasis, filariasis (e.g., lymphatic filariasis, bancroftian filariasis, subcutaneous filariasis, or serious cavity filariasis), haemonchosis (e.g., in sheep and goats, e.g., caused by Haemonchus contortus), onchocerciasis, schistosomiasis (e.g., urinary schistosomiasis, viscerial schistosomiasis, visceral schistosomiasis, acute schistosomiasis, or intestinal schistosomiasis, e.g. Asian intestinal schistosomiasis), or trichuriasis.

In some embodiments, the parasite is a fish parasite (i.e., infecting marine or freshwater fish). Like animals, fish may suffer from parastic infestations, and the parasite may be an endo- or an ectoparasites. Fish parasites include, but are not limited to, tapeworms (e.g., Schistocephalus solidus), Gyrodactylus salaris, Ichthyophthirius multifliis, cryptocaryon, velvet disease, Brooklynella hostilis, Hole in the head, Glugea, Ceratomyxa shasta, Kudoa thyrsites, Tetracapsuloides bryosalmonae, Cymothoa exigua, leeches, nematodes, flukes, and lice, such as carp lice and salmon lice. In some embodiments, the parasite is a sea louse, such as sea lice of the species Caligus or Lepeophtheirus. Sea lice are ectoparasites which feed on mucous, blood, and skin, and migrate and latch onto the skin of fish such as salmon (farm raised or wild) during their free-swimming, planktonic naupli or copepodid larval stages. In some embodiments, the compounds of the disclosure are effective against a larval stage of a parasite of the Caligus or Lepeophtheirus spp. Accordingly, in one aspect is provided a method of treating or preventing a sea lice infection in a fish, the method comprising administering to the fish an effective amount of a compound of Formula I, a salt thereof, or a composition comprising the compound or salt. In some embodiments, the fish is salmon.

In some embodiments, the parasitic disease is haemonchosis, which can be caused by infection with e.g., Haemonchus contortus (Barber's Pole worm). Haemonchus contortus is a common parasite and highly pathogenic nematode. The most common hosts are ruminants such as sheep, cattle, and goats. Haemonchosis results in anemia, edema, depression, and gastrointestinal disturbances, often leading to death of the host. The adult female worm is 18-30 millimeters and exhibits a characteristic trademark “barber pole” coloration by virtue of white ovaries coiled around the red, blood-filled intestine. The male adult worm is smaller (10-20 millimeters and displays a well-developed copulatory bursa, containing an asymmetrical dorsal lobe and a Y-shaped dorsal ray. Infection is prevalent in tropical and subtropical and warm temperate and summer rainfall regions relative to cool and cold temperate and arid regions. A current challenge in the treatment and prevention of haemonchosis is the widespread presence of Haemonchus strains resistant to the various anthelmintics, making the control of the infection difficult, affecting ruminants, causing anemia, edema, morbidity, weight loss and death of infected animals (e.g., sheep and goats).

In the Haemonchus contortus lifecycle, adult female worms in the infected host release large numbers of eggs, which are passed in the animal's feces. The eggs then develop in in the feces into the L1 (rhabditiform) and L2 juvenile stages, feeding on bacteria in the feces. The L2 rhabditform sheds its cuticle sheds its cuticle, developing into the L3 infective filiariform larvae. Sheep, goats, and other ruminants become infected when they graze and ingest the L3 larvae. The larvae pass through the first three stomach chambers to reach the abomasum, where they shed their cuticles and burrow into the internal layer of the abomasum. Once burrowed, the larvae develop into preadult larvae (L4). The L4 larvae then molt and develop into adult form (L5) where they feed on blood from the abomasum. Male and female larvae mate and produce fertilized eggs there, continuing the lifecycle.

In some embodiments, the compounds of the disclosure are effective against Haemonchus contortus. Accordingly, in one aspect is provided a method of treating or preventing haemonchosis in a ruminant, the method comprising administering to the ruminant an effective amount of a compound of Formula I, a salt thereof, or a composition comprising the compound or salt.

In some embodiments, the parasitic disease is dirofilariasis (i.e., heartworm, such as canine heartworm), which can be caused, e.g., by Dirofilaria immitis, Dirofilaria repens, or Dirofilaria honkongensis. In some embodiments, compounds of the disclosure have been found to be highly effective against Dirofilaria immitis. Accordingly, in one aspect is provided a method of treating or preventing heartworm in a companion animal, the method comprising administering to the animal an effective amount of a compound of Formula I, a salt thereof, or a composition comprising the compound or salt.

Heartworm infection is a severe and life-threatening disease. Heartworms go through several life stages before they become adults infecting the pulmonary artery of the host mammal, particularly dogs. The most common hosts are dogs and cats but other mammals such as ferrets and raccoons may also be infected. The worms require the mosquito as an intermediate host to complete their life cycle. The period between the initial infection when a host mammal (e.g., dog) is bitten by a mosquito and the maturation of the worms into adults living in the heart and pulmonary arteries is six to seven months in dogs and is known as the “prepatent period.” In the life cycle of Dirofilaria immitis, a mosquito ingests Dirofilaria immitis larvae microfilariae (first stage juvenile, L1), after a blood meal from an infected host, e.g., a dog). The L1 larvae develop into second stage larvae (L2) in the malpighian tubules of the mosquito, followed by development of third stage larvae (L3) which enter the mosquito body cavity. Development from the L1 to the L3 stage takes 15-16 days. L3 larvae migrate during blood feeding of the mosquito to the tip of the mosquito's mouth parts (labium), leave the mosquito and are deposited on the skin of the host mammal (e.g., dog), where they then migrate through the bite wound into the host. Most L3 larvae molt to fourth-stage larvae (L4) in the definitive host 0-14 days after infection. The L4 migrate to submuscular membranes and subcutaneous tissue and remain dormant. Then, they migrate to the muscles of the chest and abdomen, and 45 to 60 days after infection, molt to the fifth stage (L5, immature adult). Between 75 and 120 days after infection, these immature heartworms then enter the bloodstream and are carried through the heart to reside in the pulmonary artery. The L5 mature into adults that further migrate to the right ventricle or pulmonary artery 85-120 days after infection. The adults reach maturity in a further 2-month period, sexually reproduce in the pulmonary arteries and right ventricle, and shed microfilariae into the blood, repeating the cycle. Adult males are around 15 cm in length, and females are around 25 cm in length and their normal life span as adults is calculated to be about 5 years.

Treatment of mature heartworm infection with an adulticide (e.g., melarsomine dihydrochloride) is expensive and can cause serious adverse side effects. Accordingly, prevention by monthly administration of drugs that interrupt larvae development is widely used. The goal of marketed heartworm preventive therapies in dogs is to prevent the development of the parasite to adult heartworms by interrupting the Dirofilaria life cycle post-infection. The macrocyclic lactones (MLs; e.g., ivermectin, eprinomectin, milbemycin oxime, moxidectin, and selamectin) are the most commonly used chemoprophylaxis agents and have been effective against Dirofilaria immitis infective third-stage larvae (L3) as well as maturing fourth-stage larvae (L4). Recently, however, it has been reported that some populations of Dirofilaria immitis have developed selectional resistance to heartworm preventives (Heartworm Preventive Resistance. Is it Possible?, vol. 37. 2010 Bulletin of the American Heartworm Society, p. 5).

### Combination Therapy

In some embodiments, prevention or treatment is effected by administering a compound as provided herein along with an additional active agent as part of a combination therapy. The term “combination” in the context of combination with an additional active agent or combination therapy includes co-administration of a compound of the disclosure with an additional active agent; administration of a compound of the disclosure, followed by administration of an additional active agent; or administration of the additional active agent, followed by administration of the compound of the disclosure. Such combinations can include, e.g., administration of a single veterinary/pharmaceutical composition comprising both the compound of the disclosure and the additional active agent or the two (or more) active agents can be administered independently (e.g., as two independent compositions).

By providing such combination treatments, in some embodiments, the compounds and compositions provided herein can combat multiple pests or may control multiple lifecycle stages of certain pests. In certain embodiments, it may be advantageous to employ such compounds or compositions in combination with compounds known to be effective against other parasites or lifecycle stages thereof.

Various veterinary therapeutic agents that may be used for such combination therapies (by incorporation within a composition as provided herein or by separate administration) are known in the art. See e.g., Plumb's Veterinary Drug Handbook, 5th Edition, ed. Donald C. Plumb, Blackwell Publishing, (2005) and The Merck Veterinary Manual, 9th Edition (January 2005)), which are incorporated by reference herein in their entireties. Specific examples of suitable additional active agents include, but are not limited to, acarbose, acepromazine maleate, acetaminophen, acetazolamide, acetazolamide sodium, acetic acid, acetohydroxamic acid, acetylcysteine, acitretin, acyclovir, albendazole, albuterol sulfate, alfentanil, allopurinol, alprazolam, altrenogest, amantadine, amikacin sulfate, aminocaproic acid, aminopentamide hydrogen sulfate, aminophylline/theophylline, amiodarone, amitraz, amitriptyline, amlodipine besylate, ammonium chloride, ammonium molybdenate, amoxicillin, clavulanate potassium, amphotericin B desoxycholate, amphotericin B lipid-based, ampicillin, amprolium, antacids (oral), antivenin, apomorphione, apramycin sulfate, ascorbic acid, asparaginase, aspiring, atenolol, atipamezole, atracurium besylate, atropine sulfate, aurnofin, aurothioglucose, avermectins (e.g., abamectin, dimadectin, doramectin, emamectin, eprinomectin, ivermectin, latidectin, lepimectin, moxidectin, selamectin, and the like), azaperone, azathioprine, azithromycin, baclofen, barbituates, benazepril, betamethasone, bethanechol chloride, bisacodyl, bismuth subsalicylate, bleomycin sulfate, boldenone undecylenate, bromides, bro-mocriptine mesylate, budenoside, buprenorphine, buspirone, busulfan, butorphanol tartrate, cabergoline, calcitonin salmon, calcitrol, calcium salts, cambendazole, captopril, carbenicillin indanyl sodium, carbimazole, carboplatin, carnitine, carprofen, carvedilol, cefadroxil, cefazolin sodium, cefixime, clorsulon, cefoperazone sodium, cefotaxime sodium, cefotetan disodium, cefoxitin sodium, cefpodoxime proxetil, ceftazidime, ceftiofur sodium, ceftiofur, ceftiaxone sodium, cephalexin, cephalosporins, cephapirin, charcoal (activated), chlorambucil, chloramphenicol, chlordiazepoxide, chlordiazepoxide+/−clidinium bromide, chlorothiazide, chlorpheniramine maleate, chlorpromazine, chlorpropamide, chlortetracycline, chorionic gonadotropin (HCG), chromium, cimetidine, ciprofloxacin, cisapride, cisplatin, citrate salts, clarithromycin, clemastine fumarate, clenbuterol, clindamycin, clofazimine, clomipramine, claonazepam, clonidine, cloprostenol sodium, clorazepate dipotassium, clorsulon, cloxacillin, codeine phosphate, colchicine, corticotropin (ACTH), cosyntropin, cyclophosphamide, cyclosporine, cyproheptadine, cytarabine, dacarbazine, dactinomycin/actinomycin D, dalteparin sodium, danazol, dantrolene sodium, dapsone, decoquinate, deferoxamine mesylate, demiditraz, deracoxib, deslorelin acetate, desmopressin acetate, desoxycorticosterone pivalate, detomidine, dexamethasone, dexpanthenol, dexraazoxane, dextran, diazepam, diazoxide (oral), dichlorphenamide, diclofenac sodium, dicloxacillin, diethylcarbamazine, diethylcarbamazine citrate, diethylstilbestrol (DES), difloxacin, digoxin, dihydrotachysterol (DHT), diltiazem, dimenhydrinate, dimercaprol/BAL, dimethyl sulfoxide, dinoprost tromethamine, diphenylhydramine, disopyramide phosphate, dobutamine, docusate/DSS, dolasetron mesylate, domperidone, dopamine, doramectin, doxapram, doxepin, doxorubicin, doxycycline, edetate calcium disodium.calcium EDTA, edrophonium chloride, enalapril/enalaprilat, enoxaparin sodium, enrofloxacin, ephedrine sulfate, epinephrine, epoetin/erythropoietin, eprinomectin, epsiprantel, erythromycin, esmolol, estradiol cypionate, ethacrynic acid/ethacrynate sodium, ethanol (alcohol), etidronate sodium, etodolac, etomidate, euthanasia agents w/pentobarbital, famotidine, fatty acids (essential/omega), felbamate, fenbendazole, fentanyl, ferrous sulfate, filgrastim, finasteride, fipronil, florfenicol, flubendazole, fluconazole, flucytosine, fludrocortisone acetate, flumazenil, flumethasone, flunixin meglumine, fluorouracil (5-FU), fluoxetine, fluticasone propionate, fluvoxamine maleate, fomepizole (4-MP), furazolidone, furosemide, gabapentin, gemcitabine, gentamicin sulfate, glimepiride, glipizide, glucagon, glucocorticoid agents, glucosamine/chondroitin sulfate, glutamine, glyburide, glycerine (oral), glycopyrrolate, gonadorelin, grisseofulvin, guaifenesin, halothane, hemoglobin glutamer-200 (OXYGLOBIN®), heparin, hetastarch, hyaluronate sodium, hydrazaline, hydrochlorothiazide, hydrocodone bitartrate, hydrocortisone, hydromorphone, hydroprene, hydroxyurea, hydroxyzine, ifosfamide, imidacloprid, imidocarb dipropinate, impenem-cilastatin sodium, imipramine, inamrinone lactate, insulin, interferon alfa-2a (human recombinant), iodide (sodium/potassium), ipecac (syrup), ipodate sodium, iron dextran, isoflurane, isoproterenol, isotretinoin, isoxsuprine, itraconazole, ivermectin, kaolin/pectin, ketamine, ketoconazole, ketoprofen, ketorolac tromethamine, kinoprene, lactulose, leuprolide, levamisole, levetiracetam, levothyroxine sodium, lidocaine, lincomycin, liothyronine sodium, lisinopril, lomustine (CCNU), lufenuron, lysine, magnesium, mannitol, marbofloxacin, mebendazole, mechlorethamine, meclizine, meclofenamic acid, medetomidine, medium chain triglycerides, medroxyprogesterone acetate, megestrol acetate, melarsomine, melatonin, meloxican, melphalan, meperidine, mercaptopurine, meropenem, metaflumizone, metformin, methadone, methazolamide, methenamine mandelate/hippurate, methimazole, methionine, methocarbamol, methohexital sodium, methoprene, methotrexate, methoxyflurane, methylene blue, methylphenidate, methylprednisolone, metoclopramide, metoprolol, metronidaxole, mexiletine, mibolerlone, midazolam, milbemycins (e.g., milbemectin, milbeycin D, moxidectin, and nemadectin), milbemycin oxime, mineral oil, minocycline, misoprostol, mitotane, mitoxantrone, morantel, morphine sulfate, moxidectin, naloxone, mandrolone decanoate, naproxen, narcotic (opiate) agonist analgesics, neomycin sulfate, neostigmine, niacinamide, niclosamide, nitazoxanide, nitenpyram, nitrofurantoin, nitroglycerin, nitroprusside sodium, nizatidine, novobiocin sodium, nystatin, octadepsipeptides, octreotide acetate, olsalazine sodium, omeprozole, ondansetron, opiate antidiarrheals, orbifloxacin, oxacillin sodium, oxantel, oxazepam, oxfendazole, oxibendazole, oxibutynin chloride, 5-oxo and 5-oxime derivatives of avermectins and milbemycins, oxymorphone, oxytretracycline, oxytocin, pamidronate disodium, pancreplipase, pancuronium bromide, paraherquamides (e.g., derquantel), parbendazole, paromomycin sulfate, parozetine, pencillamine, general information penicillins, penicillin G, penicillin V potassium, pentazocine, pentobarbital sodium, pentosan polysulfate sodium, pentoxifylline, pergolide mesylate, permethrin, phenobarbital, phenoxybenzamine, pheylbutazone, phenylephrine, phenypropanolamine, phenytoin sodium, pheromones, parenteral phosphate, phytonadione/vitamin K-1, pimobendan, piperazines, pirlimycin, piroxicam, polysulfated glycosaminoglycan, ponazuril, potassium chloride, pralidoxime chloride, praziquantel, prazosin, prednisolone/prednisone, primidone, procainamide, procarbazine, prochlorperazine, propantheline bromide, propionibacterium MeCNes injection, propofol, propranolol, protamine sulfate, pseudoephedrine, psyllium hydrophilic mucilloid, pyrantel pamoate, pyrethrins, pyridostigmine bromide, pyrilamine maleate, pyrantel pamoate, pyrimethamine, pyriproxyfen, quinacrine, quinidine, ranitidine, rifampin, s-adenosyl-methionine (SAMe), saline/hyperosmotic laxative, selamectin, selegiline/1-deprenyl, sertraline, sevelamer, sevoflurane, silymarin/milk thistle, sodium bicarbonate, sodium polystyrene sulfonate, sodium stibogluconate, sodium sulfate, sodum thiosulfate, somatotropin, sotalol, spectinomycin, spironolactone, stanozolol, streptokinase, streptozocin, succimer, succinylcholine chloride, sucralfate, sufentanil citrate, sulfachlorpyridazine sodium, sulfadiazine/trimethroprim, sulfamethoxazole/trimethoprim, sulfadimentoxine, sulfadimethoxine/ormetoprim, sulfasalazine, taurine, tepoxaline, terbinafline, terbutaline sulfate, testosterone, tetracycline, tetramisole, thiabendazole, thiacetarsamide sodium, thiamine, thioguanine, thiopental sodium, thiotepa, thyrotropin, tiamulin, ticarcilin disodium, tiletamine/zolazepam, tilmocsin, tiopronin, tobramycin sulfate, tocamide, tolazoline, telfenamic acid, topiramate, tramadol, triclabendazole, trimcinolone acetonide, trientine, trilostane, trimepraxine tartrate w/prednisolone, tripelennamine, tylosin, urdosiol, valproic acid, vanadium, vancomycin, vasopressin, vecuronium bromide, verapamil, vinblastine sulfate, vincristine sulfate, vitamin E/selenium, warfarin sodium, xylazine, yohimbine, zafirlukast, zidovudine (AZT), zinc acetate/zinc sulfate, zonisamide and mixtures thereof.

Certain additional active agents that can be administered in the disclosed combination therapies are insect growth regulators (IGRs), which interfere with the development or growth of insect pests. Compounds belonging to this group are known to one of skill in the art and represent a wide range of chemical classes. Some IGRs mimic juvenile hormones or modulate levels of juvenile hormones in insects and include, but are not limited to, azadirachtin, diofenolan, fenoxycarb, hydroprene, kinoprene, methoprene, pyriproxyfen, tetrahydroazadirachtin, and 4-chloro-2(2-chloro-2-methyl-propyl)-5-(6-iodo-3-pyridylmethoxy)pyridazine-3(2H)one. Some IGRs are chitin synthesis inhibitors and include, but are not limited to, chlorofluazuron, cyromazine, diflubenzuron, fluazuron, flucycloxuron, formamidines (such as amitraz), flufenoxuron, hexaflumoron, lufenuron, novaluron, pyrethroids, tebufenozide, teflubenzuron, triflumoron, 1-(2,6-difluorobenzoyl)-3-(2-fluoro-4-(trifluoromethyl)phenylurea, 1-(2,6-difluorobenzoyl)-3-(2-fluoro-4-(1,1,2,2-tetrafluoroethoxy)-phenylurea and 1-(2,6-difluorobenzoyl)-3-(2-fluoro-4-trifluoromethyl)phenylurea.

Certain additional active agents that can be administered in the disclosed combination therapies are adulticide insecticides and acaricides. Such compounds include, e.g., pyrethrins (including cinerin I, cinerin II, jasmolin I, jasmolin II, pyrethrin I, pyrethrin II, and mixtures thereof) and pyrethroids, arylpyrazoles (e.g., fipronil, sisapronil) and carbamates (e.g., benomyl, carbanolate, cararyl, carbofuran, meththiocarb, metolcarb, promacyl, propoxyr, aldicarb, butocarboxim, oxamyl, thiocarboxime, and thiofanox).

Certain additional active agents that can be administered in the disclosed combination therapies are antinematodal agents. Suitable antinematodal agents include, but are not limited to, benzimidazoles, imidazothiazoles, tetrahydropyrimidines, and organophosphate compounds. Specific, non-limiting examples of antinematodal agents include thiabendazole, cambendazole, parbendazole, oxibendazole, mebendazole, flubendazole, fenbendazole, oxfendazole, albendazole, cyclobendazole, febantel, thiophanate, and its o,o-dimethyl analogue, tetramisole, levamisole, butamisole, pyrantel, oxantel, morantel, coumaphos, trichlorfon, haloxon, naftalofos and dichlorovos, heptenophos, mevinphos, monocrotophos, TEPP, tetrachlorvinphos, phenothiazine, piperazine as the neutral compound and in various salt forms, diethylcarbamazine, phenols such as disophenol, arsenicals such as arsenamide, ethanolamines such as bephenium, thenium closylate, and methyridine; cyanine dyes including pyrvinium chloride, pyrvinium pamoate and dithiazanine iodide; isothiocyanates including bitoscanate, suramin sodium, phthalofyne, and various natural products including, but not limited to, hygromycin B, α-santonin and kainic acid.

Certain additional active agents that can be administered in the disclosed combination therapies are antinematodal agents. Suitable antinematodal agents include, but are not limited to, the miracils such as miracil D and mirasan; praziquantel, clonazepam and its 3-methyl derivative, oltipraz, lucanthone, hycanthone, oxamniquine, amoscanate, niridazole, nitroxynil, various bisphenol compounds known in the art including hexachlorophene, bithionol, bithionol sulfoxide and menichlopholan; various salicylanilide compounds including tribromsalan, oxyclozanide, clioxanide, rafoxanide, nitroxynil, brotianide, bromoxanide and closantel; triclabendazole, diamfenetide, clorsulon, hetolin and emetine. Anticestodal compounds may also be advantageously used in certain compositions provided herein including, but not limited to, arecoline in various salt forms, bunamidine, niclosamide, nitroscanate, paromomycin, paromomycin II, praziquantel and epsiprantel.

Certain additional active agents that can be administered in the disclosed combination therapies are compounds that are effective against arthropod parasites. Such active agents include, but are not limited to, bromocyclen, chlordane, DDT, endosulfan, lindane, methoxychlor, toxaphene, bromophos, bromophosethyl, carbophenothion, chlorfenvinphos, chlorpyrifos, crotoxyphos, cythioate, diazinon, dichlorenthion, dicyclanil, diemthoate, dioxathion, ethion, famphur, fenitrothion, fenthion, fospirate, iodofenphos, malathion, naled, phosalone, phosmet, phoxim, propetamphos, ronnel, stirofos, allethrin, cyhalothrin, cypermethrin, deltamethrin, fenvalerate, flucythrinate, permethrin, phenothrin, pyrethrins, resmethrin, benzyl benzoate, carbon disulfide, crotamiton, diflubenzuron, diphenylamine, disulfiram, isobornyl thiocyanato acetate, methoprene, monosulfiram, pirenonylbutoxide, rotenone, triphenyltin acetate, triphenyltin hydroxide, deet, dimethylphthalate, and the compounds 1,5a,6,9,9a,9b-hexahydro-4a(4H)-dibenzofurancarboxaldehyde (MGK-11), 2-(2-ethylhexyl)-3a,4,7,7a-tetrahydro-4,7-methano-1H-isoindole-1,3(2H)dione (MGK-264), dipropyl-2,5-pyridinedicarboxylate (MGK-326) and 2-(octylthio)ethanol (MGK-874).

Certain additional active agents that can be administered in the disclosed combination therapies are biologically active peptides or proteins including, but not limited to, depsipeptides (e.g., including, but not limited to, emodepside), which act at the neuromuscular junction by simulating presynaptic receptors belonging to the secretin receptor family, resulting in the paralysis/death of parasites.

Certain additional active agents that can be administered in the disclosed combination therapies are from the neonicotinoid class of parasiticides, which bind and inhibit insect-specific nicotinic acetylchloline receptors. Non-limiting examples of such parasiticides include substituted pyridylmethyl derivative compounds, e.g., imidacloprid, and nitenpyram. Certain additional active agents are semicarbazones, such as metaflumizone. Certain additional active agents are isoxazoline compounds as known in the art, such as fluralaner, which may be highly effective against ectoparasites in particular. Certain additional active agents are nodulisporic acid and its derivatives (a class of known acaricidal, anthelmintic, anti-parasitic, and insecticidal agents), used to treat or prevent infections in humans and animals.

Certain additional active agents that can be administered in the disclosed combination therapies are anthelmintic compounds of the amino acetonitrile class (AAD) of compounds, e.g., monepantel (ZOLVIX®). Certain additional active agents are aryloazol-2-yl cyanoethylamino compounds and thioamide derivatives thereof. Certain additional active agents are paraherquamide compounds, which include a spirodioxepino indole core with activity against certain parasites and derivatives thereof (e.g., derquantel), as well as structurally related marcfortine compounds, such as marcfortines A-C. Certain additional active agents are spinosyns produced by the soil actinomycete Saccharopolyspora spinosa or a semi-synthetic spinosoid active agent (e.g., including, but not limited to, spinosyn A, spinosyn D, spinosad, spinetoram, or combinations thereof).

Although the technology herein has been described with reference to particular embodiments, it is to be understood that these embodiments are merely illustrative of the principles and applications of the present technology. It will be apparent to those skilled in the art that various modifications and variations can be made to the method and apparatus of the present technology without departing from the spirit and scope of the technology. Thus, it is intended that the present technology include modifications and variations that are within the scope of the appended claims and their equivalents. Accordingly, the disclosure is not limited except as by the appended claims.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Any ranges cited herein are inclusive.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

Aspects of the present technology are more fully illustrated with reference to the following examples. Before describing several exemplary embodiments of the technology, it is to be understood that the technology is not limited to the details of construction or process steps set forth in the following description. The technology is capable of other embodiments and of being practiced or being carried out in various ways. The following examples are set forth to illustrate certain aspects of the present technology and are not to be construed as limiting thereof. It is understood that one skilled in the art may be able to make these compounds by similar methods or by combining other methods known to one skilled in the art. It is also understood that one skilled in the art would be able to make, in a similar manner as described below, further compounds within the scope of the present disclosure by using appropriate starting materials and modifying the synthetic route as needed. In general, starting materials and reagents can be obtained from commercial vendors or synthesized according to sources known to those skilled in the art or prepared as described herein.

Reagent/reactant names given are as named on the commercial bottle or as generated by IUPAC conventions, ChemDraw 19.0 (CAMBRIDGESOFT®; PerkinElmer). Compounds designated as salts (e.g., hydrochloride, acetate, sulfate) may include more than one molar equivalent of the acid.

### EXEMPLIFICATION

Aspects of the present disclosure are more fully illustrated by the following examples, which are set forth to illustrate certain aspects of the present invention and are not to be construed as limiting thereof.

### Example 1. Synthesis of (S)-N-methyl-N-((4-(methylsulfonyl)morpholin-2-yl)methyl)-6-(2-azaspiro[5.5]undecan-2-yl)-2-(trifluoromethyl)pyrimidin-4-amine and (R)-N-methyl-N-((4-(methylsulfonyl)morpholin-2-yl)methyl)-6-(2-azaspiro[5.5]undecan-2-yl)-2-(trifluoromethyl)pyrimidin-4-amine (Compounds 1 and 2)

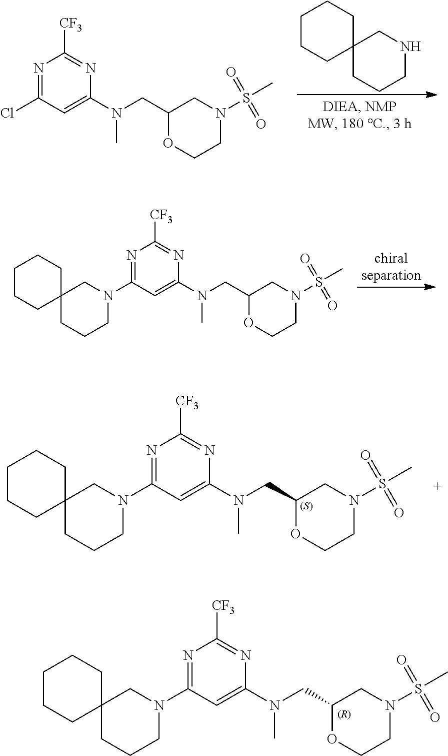

### Step A. Synthesis of N-methyl-N-((4-(methylsulfonyl)morpholin-2-yl)methyl)-6-(2-azaspiro[5.5]undecan-2-yl)-2-(trifluoromethyl)pyrimidin-4-amine

To a solution of 6-chloro-N-methyl-N-((4-(methylsulfonyl)morpholin-2-yl)methyl)-2-(trifluoromethyl)pyrimidin-4-amine (100 mg, 0.26 mmol, 1.00 equiv.) in N-methylpyrrolidone (NMP; 2 mL) was added 2-azaspiro[5.5]undecane (79 mg, 0.52 mmol, 2.00 equiv.) and diisopropylethylamine (DIEA; 99 mg, 0.77 mmol, 3.00 equiv.). The mixture was irradiated in a microwave at 180 °C for 3 h. The mixture was concentrated and purified by flash chromatography (PE/EA=20/1~10/1) to afford N-methyl-N-((4-(methylsulfonyl)morpholin-2-yl)methyl)-6-(2-azaspiro[5.5]undecan-2-yl)-2-(trifluoromethyl)pyrimidin-4-amine (180 mg, >100%) as a yellow oil. Then 180 mg of crude product was separated by Prep Chiral HPLC with the following conditions: Column: CHIRALPAK IH 3×25 cm, 5 μm; Mobile Phase A: CO₂, Mobile Phase B: IPA; Flow rate: 50 g/min; Gradient: 20% B. Wavelength: 230 nm. This afforded (S)-N-methyl-N-((4-(methylsulfonyl)morpholin-2-yl)methyl)-6-(2-azaspiro[5.5]undecan-2-yl)-2-(trifluoromethyl)pyrimidin-4-amine (37 mg, 28.3%) and (R)-N-methyl-N-((4-(methylsulfonyl)morpholin-2-yl)methyl)-6-(2-azaspiro[5.5]undecan-2-yl)-2-(trifluoromethyl)pyrimidin-4-amine (45 mg, 34.4%) as yellow solids.

Compound 1: LCMS: [M+H]⁺=506.2. ¹H NMR (400 MHz, CD₃OD): δ 1.23-1.31 (2H, m), 1.40-1.67 (12H, m), 2.64 (1H, dd, J=11.6 Hz, 10.0 Hz), 2.82 (3H, s), 2.88 (1H, td, J=11.6 Hz, 3.2 Hz), 3.10 (3H, s), 3.47 (1H, d, J=11.6 Hz), 3.52-3.66 (7H, m), 3.75-3.83 (1H, m), 3.84-3.90 (1H, m), 3.98-4.04 (1H, m), 5.68 (1H, s).

Compound 2: LCMS: [M+H]⁺=506.2. ¹H NMR (400 MHz, CD₃OD): δ 1.23-1.33 (2H, m), 1.40-1.68 (12H, m), 2.64 (1H, dd, J=11.6 Hz, 10.0 Hz), 2.82 (3H, s), 2.88 (1H, td, J=11.6 Hz, 3.2 Hz), 3.10 (3H, s), 3.47 (1H, d, J=11.6 Hz), 3.52-3.66 (7H, m), 3.76-3.83 (1H, m), 3.84-3.90 (1H, m), 3.98-4.03 (1H, m), 5.68 (1H, s).

### Example 2. Synthesis of Synthesis of (R) and (S)-N-methyl-N-((4-(methylsulfonyl)morpholin-2-yl)methyl)-6-(1-oxa-4-azaspiro[5.5]undecan-4-yl)-2-(trifluoromethyl)pyrimidin-4-amine (Compounds 3 & 4)

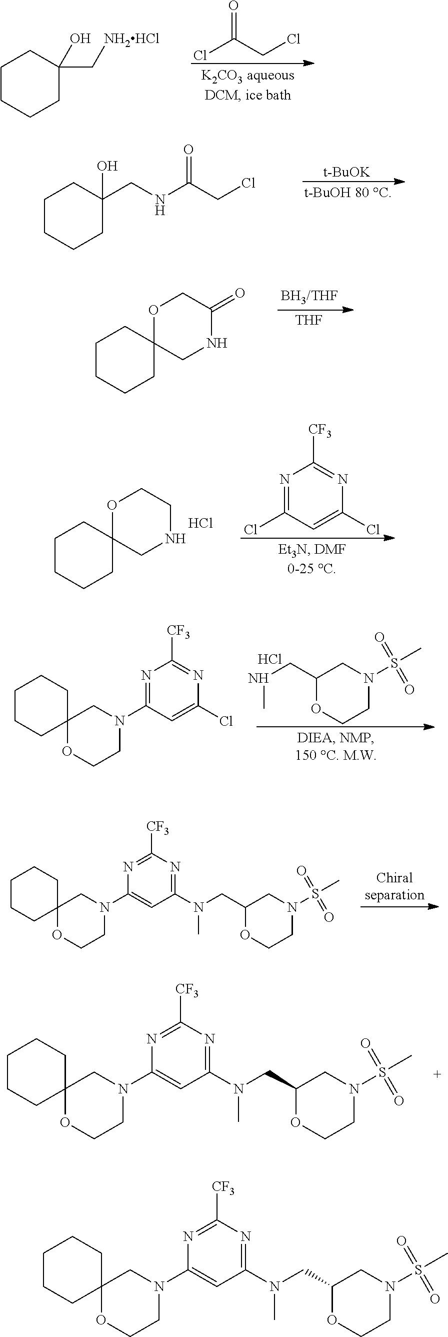

### Step A. Synthesis of 2-chloro-N-((1-hydroxycyclohexyl)methyl)acetamide

To a solution of 1-(aminomethyl)cyclohexanol hydrochloride (10 g, 60.61 mmol, 1.00 equiv.) in DCM (700 mL) was added K₂CO₃ aqueous (20.94 g, 151.52 mmol, 2.50 equiv.) and 2-chloroacetyl chloride (8.14 g, 72.73 mmol, 1.20 equiv.) at 0 °C. The mixture was stirred at 0 °C for 2 h. The reaction was diluted with DCM and washed with water and brine. The organic layer was dried over anhydrous sodium sulfate and evaporated to afford 2-chloro-N-((1-hydroxycyclohexyl)methyl)acetamide (10.99 g, 88%) as a white solid. LCMS: [M+H]⁺=206.

### Step B. Synthesis of 1-oxa-4-azaspiro[5.5]undecan-3-one

To a solution of t-BuOK (7.82 g, 69.69 mmol, 1.30 equiv) in t-BuOH (150 mL) was added 2-chloro-N-((1-hydroxycyclohexyl)methyl)acetamide (10.99 g, 53.61 mmol, 1.00 equiv.) dropwise at 80 °C, followed by stirring at 80 °C for 10 min. The solvent was evaporated, pH adjusted to 8-9 and extracted with DCM. The organic layer was dried over anhydrous sodium sulfate and evaporated to afford 1-oxa-4-azaspiro[5.5]undecan-3-one (8.50 g, 93%) as white solid. LCMS: [M+H]⁺=170.

### Step C. Synthesis of 1-oxa-4-azaspiro[5.5]undecane

A solution of 1-oxa-4-azaspiro[5.5]undecan-3-one (8.50 g, 50.00 mmol, 1.00 equiv.) in BH₃/THF (4M, 20 mL) at 0 °C was stirred at 60 °C for 16 h. MeOH and 2M HCl were added into the mixture. The reaction was stirred at R.T. for 20 min and evaporated under reduced pressure to get 1-oxa-4-azaspiro[5.5]undecane (18 g, >100%) as white solid, which was used in the next step without purification. LCMS: [M+H]⁺=156.

### Step D. Synthesis of 4-(6-chloro-2-(trifluoromethyl)pyrimidin-4-yl)-1-oxa-4-azaspiro[5.5]undecane

To a solution of crude 1-oxa-4-azaspiro[5.5]undecane (17.00 g, 11.40 mmol, 1.00 equiv.) in DCM (200 mL) was added 4,6-dichloro-2-(trifluoromethyl)pyrimidine (2.50 g, 11.40 mmol, 1.00 equiv.) and Et₃N (76 mL, 55.00 mmol, 4.80 equiv.) at 0 °C. The reaction was stirred at 25 °C for 2 h. The reaction was diluted with ethyl acetate and washed with water and brine. The organic layer was dried over anhydrous sodium sulfate, filtered, and concentrated to obtain crude product. The crude product was purified by flash chromatography (EA/PE=8%) to afford 4-(6-chloro-2-(trifluoromethyl)pyrimidin-4-yl)-1-oxa-4-azaspiro[5.5]undecane (4.67 g, 27.8%) as white solid. LCMS: [M+H]⁺=336.

### Step E. Synthesis of N-methyl-N-((4-(methylsulfonyl)morpholin-2-yl)methyl)-6-(1-oxa-4-azaspiro[5.5]undecan-4-yl)-2-(trifluoromethyl)pyrimidin-4-amine

To a solution of 4-(6-chloro-2-(trifluoromethyl)pyrimidin-4-yl)-1-oxa-4-azaspiro[5.5]undecane (100 mg, 0.30 mmol, 1.00 equiv) and DIEA (97 mg, 0.75 mmol, 2.50 equiv) in NMP (2 mL) was added N-methyl-1-(4-(methylsulfonyl)morpholin-2-yl)methanamine HCl (145 mg, 0.60 mmol, 2.00 equiv). The mixture was stirred at 150 °C for 2 h in microwave. The mixture was concentrated. The residue was purified by Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 60% B to 80% B in 10 min, Wavelength: 214 nm. This afforded N-methyl-N-((4-(methylsulfonyl)morpholin-2-yl)methyl)-6-(1-oxa-4-azaspiro[5.5]undecan-4-yl)-2-(trifluoromethyl)pyrimidin-4-amine (97 mg, 63%). The residue was separated by using Chiral Prep-HPLC to afford Compound 3 (25 mg) and Compound 4 (29 mg) as white solids.

Compound 3: LCMS: [M+H]⁺=508.2. 1H NMR (400 MHz, CDCl3): δ 1.22-1.53 (7H, m), 1.60-1.80 (3H, m), 2.60-2.67 (1H, m), 2.74 (3H, s), 2.77-2.82 (1H, m), 3.04 (3H, s), 3.40-3.51 (3H, m), 3.52-3.61 (4H, m), 3.62-3.69 (1H, m), 3.72-3.80 (3H, m), 3.96-4.10 (2H, m), 5.39 (1H, s).

Compound 4: LCMS: [M+H]⁺=508.2. ¹H NMR (400 MHz, CDCl₃): δ 1.30-1.50 (7H, m), 1.59-1.76 (3H, m), 2.60-2.67 (1H, m), 2.74 (3H, s), 2.76-2.85 (1H, m), 3.04 (3H, s), 3.44-3.51 (3H, m), 3.52-3.61 (4H, m), 3.62-3.69 (1H, m), 3.72-3.81 (3H, m), 3.96-4.10 (2H, m), 5.39 (1H, s).

### Example 3. Synthesis of 4-(((6-(2-azaspiro[5.5]undecan-2-yl)-2-(trifluoromethyl)pyrimidin-4-yl)(methyl)amino)methyl)-N,N-dimethylbenzenesulfonamide (Compound 5)

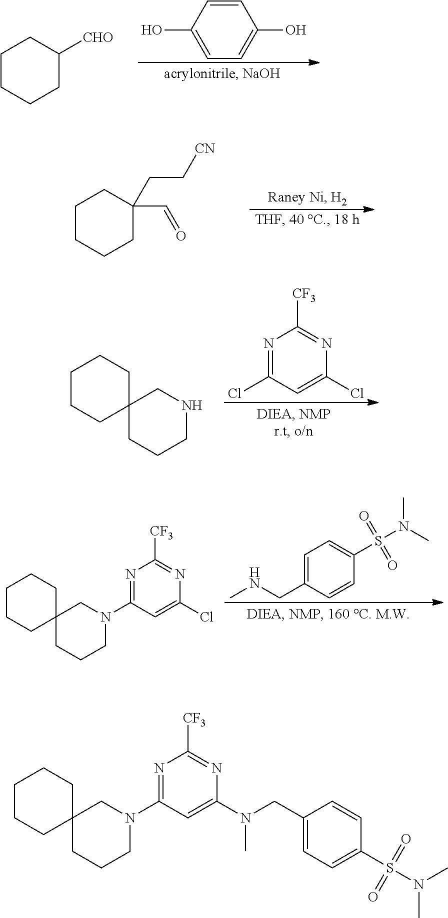

### Step A. Synthesis of 3-(1-formylcyclohexyl)propanenitrile

To a stirred solution of cyclohexanecarbaldehyde (2.50 g, 22.32 mmol, 1.00 equiv), hydroquinone (12 mg, 0.11 mmol, 0.005 equiv) and acrylonitrile (2.85 mL, 27.90 mmol, 1.25 equiv) in 1,4-dioxane (13 mL) were added 5% NaOH aq (2.3 mL) and stirred at 65 °C for 2.5 h. The resulting mixture was stirred at 25 °C for 16 h. The mixture was concentrated under reduced pressure. The crude was purified by flash chromatography (PE/EA=20/1) to afford 3-(1-formylcyclohexyl)propanenitrile (2.60 g, 71%) as a colorless oil.

### Step B. Synthesis of 2-azaspiro[5.5]undecane

To a stirred solution of 3-(1-formylcyclohexyl)propanenitrile (2.30 g, 13.90 mmol, 1.00 equiv) in THF (120 mL) were added Raney nickel (1.00 g) at room temperature. The resulting mixture was stirred at 40 °C for 48 h. The mixture was filtered and The mixture was concentrated under reduced pressure to afford 2-azaspiro[5.5]undecane (2.00 g, 95%) as a yellow oil. LCMS: [M+H]⁺=154.

### Step C. Synthesis of 2-(6-chloro-2-(trifluoromethyl)pyrimidin-4-yl)-2-azaspiro[5.5]undecane

To a stirred solution of 4,6-dichloro-2-(trifluoromethyl)pyrimidine (0.71 g, 3.27 mmol, 1.00 equiv) and 2-azaspiro[5.5]undecane (0.50 g, 3.27 mmol, 1.00 equiv) in NMP (8 mL) was added DIEA (1.26 g, 9.80 mmol, 3.00 equiv). The resulting mixture was stirred at 30 °C for 16 h. The mixture was concentrated under reduced pressure. The crude was purified by flash chromatography (PE/EA=20/1) to afford N-methyl-4-(methylsulfonyl)morpholine-2-carboxamide (0.32 g, 29%) as a white solid. LCMS: [M+H]⁺=334.

### Step D. Synthesis of 4-(((6-(2-azaspiro[5.5]undecan-2-yl)-2-(trifluoromethyl)pyrimidin-4-yl)(methyl)amino)methyl)-N,N-dimethylbenzenesulfonamide

To a solution of N,N-dimethyl-4-((methylamino)methyl)benzenesulfonamide (114 mg, 0.50 mmol, 1.00 equiv) in NMP (2 mL) were added DIEA (194 mg, 1.50 mmol, 3.00 equiv) and 2-(6-chloro-2-(trifluoromethyl)pyrimidin-4-yl)-2-azaspiro[5.5]undecane (167 mg, 0.50 mmol, 1.00 equiv). The reaction mixture was stirred at 160 °C for 3 hours in microwave. The mixture was added to water, extracted with EtOAc (10 mL×3). The organic layer was washed with brine, dried over Na₂SO₄ and concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 80% B to 90% B in 10 min, Wavelength: 214 nm. This afforded the title compound (32 mg, 12.2%) as white solid. LCMS: [M+H]⁺=526.2. ¹H NMR (400 MHz, CDCl₃): δ 1.21-1.28 (2H, m), 1.33-1.38 (4H, m), 1.42-1.50 (6H, m), 1.60-1.63 (2H, m), 2.70 (6H, s), 3.00 (3H, s), 3.46 (2H, s), 3.52 (2H, J=5.60 Hz, t), 4.90 (2H, s), 5.45 (1H, s), 7.40 (2H, J=8.40, d), 7.72 (2H, J=8.40, d).

### Example 4. Synthesis of N-methyl-N-(4-(methylsulfonyl)benzyl)-6-(2-azaspiro[5.5]undecan-2-yl)-2-(trifluoromethyl)pyrimidin-4-amine (Compound 6)

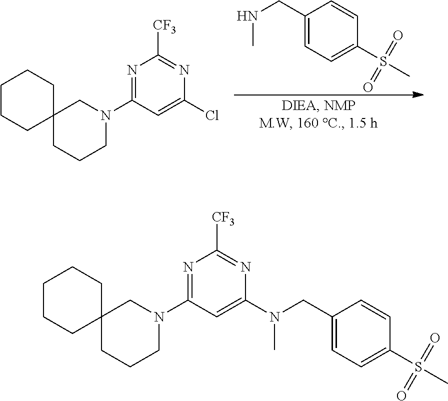

To a stirred solution of 2-(6-chloro-2-(trifluoromethyl)pyrimidin-4-yl)-2-azaspiro[5.5]undecane (50 mg, 0.15 mmol, 1.00 equiv) in NMP (2 mL) was added DIEA (58 mg, 0.45 mmol, 3.00 equiv) and N-methyl-1-(4-(methylsulfonyl)phenyl)methanamine (45 mg, 0.23 mmol, 1.50 equiv). The resulting mixture was stirred at 160 °C for 1.5 h under Microwave. The reaction mixture was purified using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 65% B to 90% B in 12 min; Wavelength: 214 nm. This afforded the title compound (15 mg, 20%) as a white solid. LCMS: [M+H]⁺=497.2. ¹HNMR (400 MHz, DMSO-d₆): δ 1.14-1.51 (14H, m), 3.05 (3H, s), 3.18 (3H, s), 3.47-3.54 (4H, m), 4.89 (2H, s), 5.81 (1H, s), 7.47 (2H, d, J=8.4 Hz), 7.88 (2H, d, J=8.4 Hz).

### Example 5. Synthesis of N-(6-((6-(2-azaspiro[5.5]undecan-2-yl)-2-(trifluoromethyl)pyrimidin-4-yl)(methyl)amino)spiro[3.3]heptan-2-yl)methanesulfonamide (Compound 7)

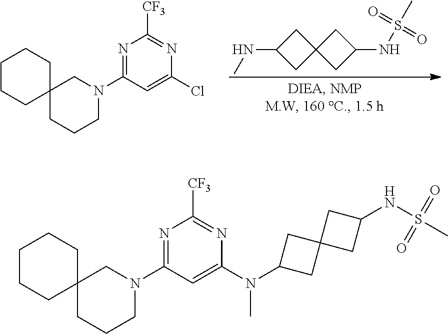

To a stirred solution of 2-(6-chloro-2-(trifluoromethyl)pyrimidin-4-yl)-2-azaspiro[5.5]undecane (100 mg, 0.30 mmol, 1.00 equiv) in NMP (2 mL) was added DIEA (116 mg, 0.90 mmol, 3.00 equiv) and N-(6-(methylamino)spiro[3.3]heptan-2-yl)methanesulfonamide (98 mg, 0.45 mmol, 1.50 equiv). The resulting mixture was stirred at 160 °C for 1.5 h under Microwave. The reaction mixture was purified using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 55% B to 80% B in 12 min; Wavelength: 214 nm. This afforded the title compound (20 mg, 13%) as a white solid. LCMS: [M+H]⁺=516.3. ¹HNMR (400 MHz, DMSO-d₆): δ 1.12-1.52 (14H, m), 1.94-2.36 (8H, m), 2.83 (3H, s), 2.89 (3H, s), 3.50-3.52 (4H, m), 3.62-3.69 (1H, m), 4.69-4.73 (1H, m), 5.68 (1H, s), 7.30 (1H, d, J 8.4 Hz).

### Example 6. Synthesis of N-methyl-N-(2-phenoxyethyl)-6-(2-azaspiro[5.5]undecan-2-yl)-2-(trifluoromethyl)pyrimidin-4-amine (Compound 8)

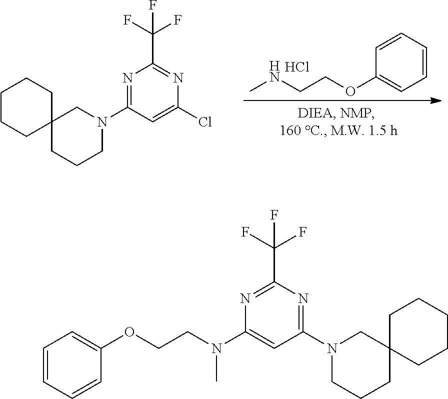 [SMILES: FC(F)(F)c1nc(Cl)cc(N2CCCC3(CCCCC3)C2)n1 ; CNCCOc1ccccc1 ; Cl ; CN(CCOc1ccccc1)c1cc(N2CCCC3(CCCCC3)C2)nc(C(F)(F)F)n1]

To a stirred solution of 2-(6-chloro-2-(trifluoromethyl)pyrimidin-4-yl)-2-azaspiro[5.5]undecane (50 mg, 0.15 mmol, 1.00 equiv) in NMP (2 mL) was added DIEA (58 mg, 0.45 mmol, 3.00 equiv) and N-methyl-2-phenoxyethan-1-amine hydrochloride (42 mg, 2.25 mmol, 1.5 equiv). The resulting mixture was stirred at 160 °C for 1.5 h under Microwave. To the mixture was added water (20 mL), extracted with EtOAc (10 mL). The combined organic layer was washed with brine and concentrated in vacuo. The residue was purified using Prep-HPLC with the following conditions: Column: XBridge Prep C18 Column, 19×150 mm, 5 μm; Mobile Phase A: Water (0.1% NH4HCO3), Mobile Phase B: ACN; Flow rate: 15 mL/min; Gradient: 80% B to 95% B in 12 min; Wavelength: 214 nm. This afforded the title compound (35 mg, 52%) as a white solid. LCMS: [M+H]⁺=449.3. ¹HNMR (400 MHz, CDCl₃-d): δ 1.20-1.52 (12H, m), 1.58-1.62 (2H, m), 3.12 (3H, s), 3.47-3.53 (4H, m), 3.95-3.98 (2H, m), 4.15-4.18 (2H, m), 5.43 (1H, s), 6.91-6.95 (3H, m), 7.26-7.29 (2H, m).

### Example 7. Synthesis of N-methyl-N-((1-(methylsulfonyl)pyrrolidin-3-yl)methyl)-6-(2-azaspiro[5.5]undecan-2-yl)-2-(trifluoromethyl)pyrimidin-4-amine (Compound 9)

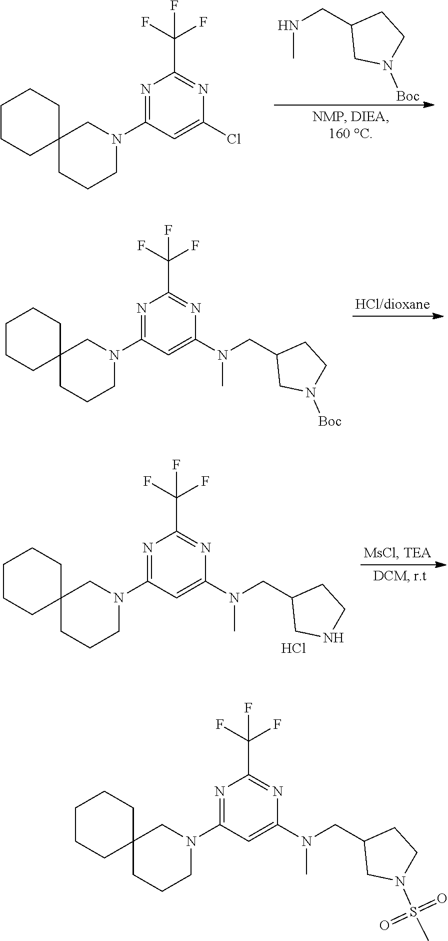 [SMILES: FC(F)(F)c1nc(Cl)cc(N2CCCC3(CCCCC3)C2)n1 ; CNCC1CCN(C(=O)OC(C)(C)C)C1 ; CN(CC1CCN(C(=O)OC(C)(C)C)C1)c1cc(N2CCCC3(CCCCC3)C2)nc(C(F)(F)F)n1 ; CN(CC1CCNC1)c1cc(N2CCCC3(CCCCC3)C2)nc(C(F)(F)F)n1 ; Cl ; CN(CC1CCN(S(C)(=O)=O)C1)c1cc(N2CCCC3(CCCCC3)C2)nc(C(F)(F)F)n1]

### Step A. Synthesis of tert-butyl 3-(((6-(2-azaspiro[5.5]undecan-2-yl)-2-(trifluoromethyl)pyrimidin-4-yl)(methyl)amino)methyl)pyrrolidine-1-carboxylate

To a solution of 2-(6-chloro-2-(trifluoromethyl)pyrimidin-4-yl)-2-azaspiro[5.5]undecane (333 mg, 1.00 mmol, 1.00 equiv) in NMP (5 mL) was added DIEA (258 mg, 2.00 mmol, 2.00 equiv) and tert-butyl 3-((methylamino)methyl)pyrrolidine-1-carboxylate (214 mg, 1.00 mmol, 1.00 equiv) at 25 °C. The reaction mixture was stirred at 160 °C for 2 hours. Then the organic layer was concentrated in vacuo and the residue was purified by Flash chromatography (EA/PE=10%) to afford tert-butyl 3-(((6-(2-azaspiro[5.5]undecan-2-yl)-2-(trifluoromethyl)pyrimidin-4-yl)(methyl)amino)methyl)pyrrolidine-1-carboxylate (420 mg, 82%) as yellow oil. LCMS: [M+H]⁺=512.

### Step B. Synthesis of N-methyl-N-(pyrrolidin-3-ylmethyl)-6-(2-azaspiro[5.5]undecan-2-yl)-2-(trifluoromethyl)pyrimidin-4-amine (HCl Salt)

A solution of tert-butyl 3-(((6-(2-azaspiro[5.5]undecan-2-yl)-2-(trifluoromethyl)pyrimidin-4-yl)(methyl)amino)methyl)pyrrolidine-1-carboxylate (420 mg, 0.82 mmol, 1.00 equiv) in 4 M HCl/dioxane (8 mL) was stirred at 25 °C for 1 hour. Then the solvents were removed in vacuo and the residue was dried in vacuo to get the N-methyl-N-(pyrrolidin-3-ylmethyl)-6-(2-azaspiro[5.5]undecan-2-yl)-2-(trifluoromethyl)pyrimidin-4-amine (520 mg, 100%) as yellow solid, which was used directly in the next step without further purification. LCMS: [M+H]⁺=412.

### Step C. Synthesis of N-methyl-N-((1-(methylsulfonyl)pyrrolidin-3-yl)methyl)-6-(2-azaspiro[5.5]undecan-2-yl)-2-(trifluoromethyl)pyrimidin-4-amine

To a solution of N-methyl-N-(pyrrolidin-3-ylmethyl)-6-(2-azaspiro[5.5]undecan-2-yl)-2-(trifluoromethyl)pyrimidin-4-amine (270 mg, 0.60 mmol, 1.00 equiv) in DCM (5 mL) were added TEA (182 mg 1.80 mmol, 3.00 equiv) and MsCl (69 mg, 0.60 mmol, 1.00 equiv). The reaction mixture was stirred at 25 °C for 1 hour. The mixture was extracted with EtOAc (20 mL×3) and washed with brine. The organic layer was dried over Na₂SO₄ and concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 70% B to 80% B in 11 min, Wavelength: 214 nm. This afforded the title compound (46 mg, 16%) as white solid. LCMS: [M+H]⁺=490.2. ¹H NMR (400 MHz, CDCl₃): δ 1.24-1.27 (2H, m), 1.35-1.51 (10H, m), 1.58-1.63 (2H, m), 1.73-1.75 (1H, m), 2.01-2.03 (1H, m), 2.66-2.69 (1H, m), 2.83 (3H, s), 2.98 (3H, s), 3.08-3.12 (1H, m), 3.35-3.54 (5H, m), 3.59-3.61 (2H, m), 3.63-3.73 (2H, in)), 5.39 (1H, s).

### Example 8. Synthesis of 6-(6-(2-azaspiro[5.5]undecan-2-yl)-2-(trifluoromethyl)pyrimidin-4-yl)-N,N-dimethyl-2,6-diazaspiro[3.3]heptane-2-sulfonamide (Compound 10)

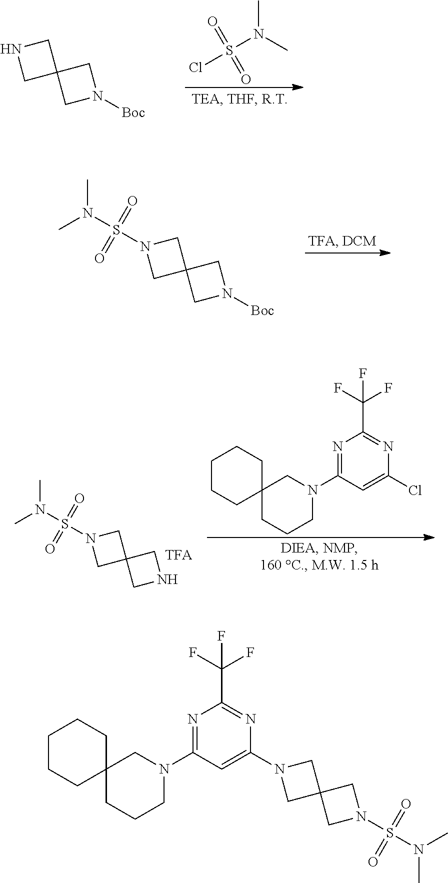

### Step A. Synthesis of tert-butyl 6-(N,N-dimethylsulfamoyl)-2,6-diazaspiro[3.3]heptane-2-carboxylate

To a solution of tert-butyl 2,6-diazaspiro[3.3]heptane-2-carboxylate (300 mg, 1.52 mmol, 1.00 equiv) in THF (10 mL) was added TEA (307 mg, 3.04 mmol, 2.0 equiv) and dimethylsulfamoyl chloride (260 mg, 1.82 mmol, 1.2 equiv). The mixture was stirred at R.T. for 16 h. The mixture was added water, extracted with EtOAc. The combined organic was dried over Na₂SO₄, filtered and concentrated to give the crude tert-butyl 6-(N,N-dimethylsulfamoyl)-2,6-diazaspiro[3.3]heptane-2-carboxylate (480 mg, >100%) as a white solid. LCMS: [M+H-56]⁺=250.

### Step B. Synthesis of N,N-dimethyl-2,6-diazaspiro[3.3]heptane-2-sulfonamide

To a solution of tert-butyl 6-(N,N-dimethylsulfamoyl)-2,6-diazaspiro[3.3]heptane-2-carboxylate (200 mg, 0.59 mmol, 90% wt, 1.00 equiv) in DCM (8 mL) was added TFA (2 mL). The mixture was stirred at R.T. for 2 h. The mixture was concentrated in vacuo to get the crude N,N-dimethyl-2,6-diazaspiro[3.3]heptane-2-sulfonamide (300 mg, >100%) as a colorless oil. LCMS: [M+H]⁺=206.

### Step C. Synthesis of 6-(6-(2-azaspiro[5.5]undecan-2-yl)-2-(trifluoromethyl)pyrimidin-4-yl)-N,N-dimethyl-2,6-diazaspiro[3.3]heptane-2-sulfonamide

To a stirred solution of 2-(6-chloro-2-(trifluoromethyl)pyrimidin-4-yl)-2-azaspiro[5.5]undecane (80 mg, 0.24 mmol, 1.00 equiv) in NMP (2 mL) was added DIEA (93 mg, 0.72 mmol, 3.00 equiv) and N,N-dimethyl-2,6-diazaspiro[3.3]heptane-2-sulfonamide (crude 115 mg, 0.36 mmol, 1.5 equiv). The resulting mixture was stirred at 160 °C for 1.5 h under Microwave. To the mixture was added water (20 mL), extracted with EtOAc (10 mL). The combined organic layer was washed with brine and concentrated in vacuo. The residue was purified using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 65% B to 90% B in 10 min; Wavelength: 214 nm. This afforded the title compound (45 mg, 37%) as a white solid. LCMS: [M+H]⁺=503.1. ¹HNMR (400 MHz, CDCl₃): δ 1.20-1.49 (13H, m), 1.50-1.52 (1H, m), 2.81 (6H, s), 3.45-3.51 (4H, m), 4.05 (4H, s), 4.16 (4H, s), 5.43 (1H, s).

### Example 9. Synthesis of 2-(2-((6-(2-azaspiro[5.5]undecan-2-yl)-2-(trifluoromethyl)pyrimidin-4-yl)(methyl)amino)ethyl)isothiazolidine 1,1-dioxide (Compound 11)

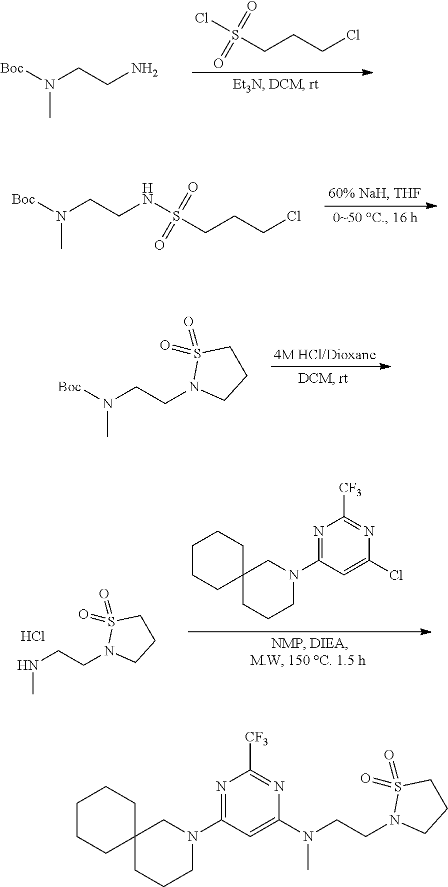

### Step A. Synthesis of tert-butyl (2-((3-chloropropyl)sulfonamido)ethyl)(methyl)carbamate

To a solution of tert-butyl (2-aminoethyl)(methyl)carbamate (500 mg, 2.87 mmol, 1.00 equiv) and TEA (575 mg, 5.75 mmol, 2.00 equiv) in DCM (10 mL) was slowly added 3-chloropropane-1-sulfonyl chloride (505 mg, 2.87 mmol, 1.00 equiv) at 0 °C. The mixture was stirred at room temperature for 16 h. The mixture was extracted with EtOAc and washed with water. The organic layer was dried over Na₂SO₄ and concentrated. This afforded crude tert-butyl (2-((3-chloropropyl)sulfonamido)ethyl)(methyl)carbamate (724 mg, 80%) as yellow oil. LCMS: [M+H-100]⁺=215.

### Step B. Synthesis of tert-butyl (2-(1,1-dioxidoisothiazolidin-2-yl)ethyl)(methyl)carbamate

To a solution of crude tert-butyl (2-((3-chloropropyl)sulfonamido)ethyl)(methyl)carbamate (600 mg, 1.91 mmol, 1.00 equiv) in THF (10 mL) was slowly added 60% NaH (153 mg, 3.82 mmol, 2.00 equiv). The reaction mixture was stirred at 0 °C for 30 min. Then the mixture was stirred at 50 °C for 16 h. The mixture was extracted with EtOAc and washed with brine. The organic layer was dried over Na₂SO₄ and concentrated. It was given the crude tert-butyl (2-(1,1-dioxidoisothiazolidin-2-yl)ethyl)(methyl)carbamate (600 mg, >100%) as yellow oil. LCMS: [M+H-56]⁺=223.

### Step C. Synthesis of 2-(2-(methylamino)ethyl)isothiazolidine 1,1-dioxide (HCl Salt)

To a solution of crude tert-butyl (2-(1,1-dioxidoisothiazolidin-2-yl)ethyl)(methyl)carbamate (600 mg, 2.16 mmol, 1.00 equiv) in DCM (10 mL) was added 4 mL HCl (4M in 1,4-dioxane). The mixture was stirred at room temperature for 2 h. The mixture was concentrated to get crude 2-(2-(methylamino)ethyl)isothiazolidine 1,1-dioxide (HCl salt) (540 mg, >100%) as off-white solid. LCMS: [M+H]⁺=179

### Step D. Synthesis of 2-(2-((6-(2-azaspiro[5.5]undecan-2-yl)-2-(trifluoromethyl)pyrimidin-4-yl)(methyl)amino)ethyl)isothiazolidine 1,1-dioxide

To a solution of crude 2-(6-chloro-2-(trifluoromethyl)pyrimidin-4-yl)-2-azaspiro[5.5]undecane (45 mg, 0.14 mmol, 1.00 equiv) and 2-(2-(methylamino)ethyl)isothiazolidine 1,1-dioxide (HCl salt) (90 mg, 0.42 mmol, 3.00 equiv) in NMP (2 mL) was added DIEA (72 mg, 0.56 mmol, 4.00 equiv). The mixture was stirred at 150 °C for 1.5 h in a microwave. The mixture was extracted with EtOAc and washed with brine. The organic layer was dried over Na₂SO₄ and concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 70% B to 90% B in 10 min. Wavelength: 214 nm. This afforded the title compound (70 mg, 87%) as white solid. LCMS: [M+H]⁺=476.2. ¹H NMR (400 MHz, DMSO-d6): δ 1.14-1.19 (2H, m), 1.31-1.52 (12H, m), 2.12-2.18 (2H, m), 3.01 (3H, s), 3.05-3.13 (4H, m), 3.27 (2H, t, J=6.4 Hz), 3.50-3.54 (4H, m), 3.69-3.72 (2H, m), 5.75 (1H, s).

### Example 10. Synthesis of N-(6-(2-azaspiro[5.5]undecan-2-yl)-2-(trifluoromethyl)pyrimidin-4-yl)-2-(methylsulfonyl)-2-azaspiro[3.3]heptan-6-amine (Compound 12)

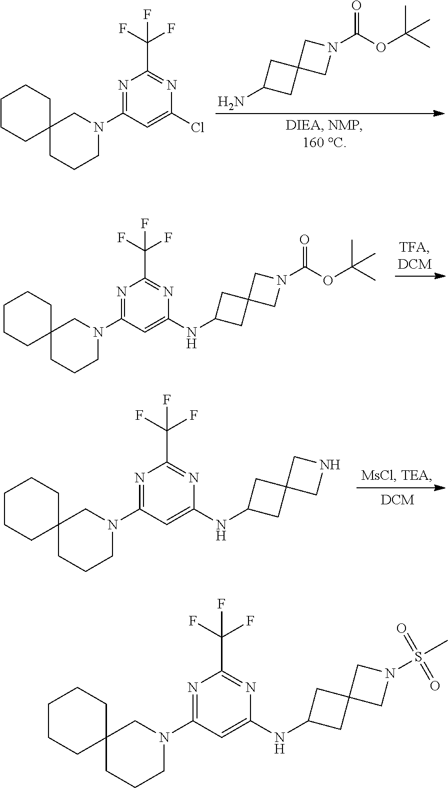 [SMILES: FC(F)(F)c1nc(Cl)cc(N2CCCC3(CCCCC3)C2)n1 ; CC(C)(C)OC(=O)N1CC2(CC(N)C2)C1 ; CC(C)(C)OC(=O)N1CC2(CC(Nc3cc(N4CCCC5(CCCCC5)C4)nc(C(F)(F)F)n3)C2)C1 ; FC(F)(F)c1nc(NC2CC3(CNC3)C2)cc(N2CCCC3(CCCCC3)C2)n1 ; CS(=O)(=O)N1CC2(CC(Nc3cc(N4CCCC5(CCCCC5)C4)nc(C(F)(F)F)n3)C2)C1]

### Step A. Synthesis of tert-butyl 6-((6-(2-azaspiro[5.5]undecan-2-yl)-2-(trifluoromethyl)pyrimidin-4-yl)amino)-2-azaspiro[3.3]heptane-2-carboxylate

To a solution of 2-(6-chloro-2-(trifluoromethyl)pyrimidin-4-yl)-2-azaspiro[5.5]undecane (90 mg, 0.27 mmol, 1.00 equiv) in NMP (2 mL) were added DIEA (104 mg, 0.81 mmol, 3.00 equiv) and tert-butyl 6-amino-2-azaspiro[3.3]heptane-2-carboxylate (57 mg, 0.27 mmol, 1.00 equiv). The reaction mixture was stirred at 160 °C for 6 hours in microwave. The mixture was extracted with EtOAc (10 mL×3) and washed with brine. The organic layer was dried over Na₂SO₄ and concentrated. The residue was purified by SGC (PE/EA=5/1) to get tert-butyl 6-((6-(2-azaspiro[5.5]undecan-2-yl)-2-(trifluoromethyl)pyrimidin-4-yl)amino)-2-azaspiro[3.3]heptane-2-carboxylate (50 mg, 37%) as yellow solid. LCMS: [M+H]⁺=510.

### Step B. Synthesis of N-(6-(2-azaspiro[5.5]undecan-2-yl)-2-(trifluoromethyl)pyrimidin-4-yl)-2-azaspiro[3.3]heptan-6-amine

A solution of tert-butyl 6-((6-(2-azaspiro[5.5]undecan-2-yl)-2-(trifluoromethyl)pyrimidin-4-yl)amino)-2-azaspiro[3.3]heptane-2-carboxylate (50 mg, 0.10 mmol, 1.00 equiv) in DCM (2 mL) was added TFA (1 mL), stirred at 25 °C for 1 hour. The mixture was concentrated under vacuum to get N-(6-(2-azaspiro[5.5]undecan-2-yl)-2-(trifluoromethyl)pyrimidin-4-yl)-2-azaspiro[3.3]heptan-6-amine crude (130 mg, >100%) as yellow oil, which was used directly in the next step without further purification. LCMS: [M+H]⁺=410.2.

### Step C. Synthesis of N-(6-(2-azaspiro[5.5]undecan-2-yl)-2-(trifluoromethyl)pyrimidin-4-yl)-2-(methylsulfonyl)-2-azaspiro[3.3]heptan-6-amine

To a solution of N-(6-(2-azaspiro[5.5]undecan-2-yl)-2-(trifluoromethyl)pyrimidin-4-yl)-2-azaspiro[3.3]heptan-6-amine (130 mg, 0.10 mmol, 1.00 equiv) in DCM (5 mL) was added TEA (51 mg, 0.50 mmol, 5.00 equiv) and MsCl (14 mg, 0.12 mmol, 1.20 equiv) at 25 °C. The reaction mixture was stirred at 25 °C for 2 hours. Then the mixture was extracted with DCM (20 mL×3), washed by brine. The organic layer was dried over Na₂SO₄ and concentrated. The residue was purified by prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 70% B to 80% B in 10 min, Wavelength: 214 nm. This afforded the title compound (7 mg, 14% yield) as white solid. LCMS: [M+H]⁺=488.1. ¹H NMR (400 MHz, DMSO-d₆): δ 1.13-1.52 (16H, m), 2.06-2.11 (2H, m), 2.56-2.61 (2H, m), 2.95 (3H, s), 3.45-3.47 (3H, m), 3.80 (2H, s), 3.93 (2H, s), 5.54 (1H, s), 7.44 (1H, s).

### Example 11. Synthesis of N-methyl-N-((1-(methylsulfonyl)azetidin-3-yl)methyl)-6-(2-azaspiro[5.5]undecan-2-yl)-2-(trifluoromethyl)pyrimidin-4-amine (Compound 13)

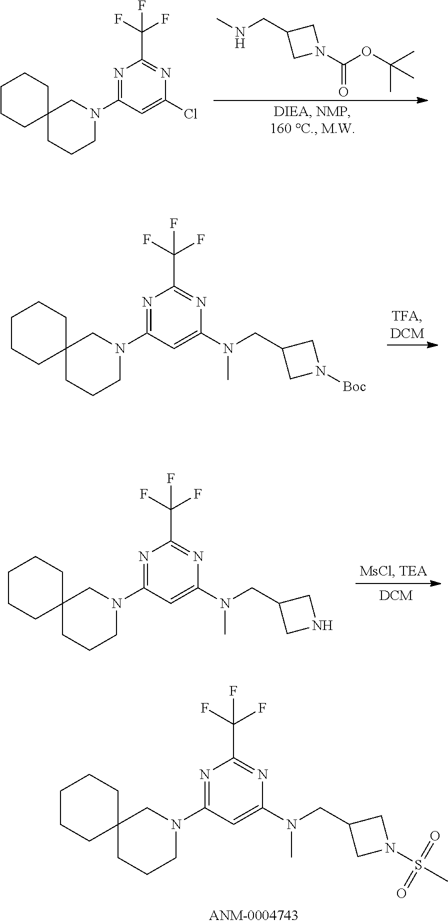 [SMILES: FC(F)(F)c1nc(Cl)cc(N2CCCC3(CCCCC3)C2)n1 ; CNCC1CN(C(=O)OC(C)(C)C)C1 ; CN(CC1CN(C(=O)OC(C)(C)C)C1)c1cc(N2CCCC3(CCCCC3)C2)nc(C(F)(F)F)n1 ; CN(CC1CNC1)c1cc(N2CCCC3(CCCCC3)C2)nc(C(F)(F)F)n1 ; CN(CC1CN(S(C)(=O)=O)C1)c1cc(N2CCCC3(CCCCC3)C2)nc(C(F)(F)F)n1]

### Step A. Synthesis of tert-butyl 3-(((6-(2-azaspiro[5.5]undecan-2-yl)-2-(trifluoromethyl)pyrimidin-4-yl)(methyl)amino)methyl)azetidine-1-carboxylate

To a solution of tert-butyl 3-((methylamino)methyl)azetidine-1-carboxylate (72 mg, 0.36 mmol, 1.00 equiv) in NMP (5 mL) was added DIEA (93 mg, 0.72 mmol, 2.00 equiv) and 2-(6-chloro-2-(trifluoromethyl)pyrimidin-4-yl)-2-azaspiro[5.5]undecane (120 mg, 0.36 mmol, 1.00 equiv) at R.T. and the reaction mixture was stirred at 160 °C for 3 hour in microwave. Then the mixture was extracted with EtOAc (20 mL×3), washed by brine. The organic layer was dried over Na₂SO₄ and concentrated to get the tert-butyl 3-(((6-(2-azaspiro[5.5]undecan-2-yl)-2-(trifluoromethyl)pyrimidin-4-yl)(methyl)amino)methyl)azetidine-1-carboxylate (179 mg, 100% yield) as yellow oil, which was used directly in the next step without further purification. LCMS: [M+H]⁺=498.

### Step B. Synthesis of N-(azetidin-3-ylmethyl)-N-methyl-6-(2-azaspiro[5.5]undecan-2-yl)-2-(trifluoromethyl)pyrimidin-4-amine

A solution of tert-butyl 3-(((6-(2-azaspiro[5.5]undecan-2-yl)-2-(trifluoromethyl)pyrimidin-4-yl)(methyl)amino)methyl)azetidine-1-carboxylate (179 mg, 0.36 mmol, 1.00 equiv) in TFA/DCM (3/3 mL) was stirred at 25 °C for 1 hour. Then the solution was removed in vacuo and the residue was concentrated in vacuo to get the N-(azetidin-3-ylmethyl)-N-methyl-6-(2-azaspiro[5.5]undecan-2-yl)-2-(trifluoromethyl)pyrimidin-4-amine crude (200 mg, 100% yield) as brown oil, which was used directly in the next step without further purification. LCMS: [M+H]⁺=398.

### Step C. Synthesis of N-methyl-N-((1-(methylsulfonyl)azetidin-3-yl)methyl)-6-(2-azaspiro[5.5]undecan-2-yl)-2-(trifluoromethyl)pyrimidin-4-amine

To a solution of N-(azetidin-3-ylmethyl)-N-methyl-6-(2-azaspiro[5.5]undecan-2-yl)-2-(trifluoromethyl)pyrimidin-4-amine N-(azetidin-3-ylmethyl)-N-methyl-6-(2-azaspiro[5.5]undecan-2-yl)-2-(trifluoromethyl)pyrimidin-4-amine (200 mg, 0.50 mmol, 1.00 equiv) in DCM (5 mL) were added TEA (151 mg, 1.50 mmol, 3.00 equiv) and MsCl (58 mg, 0.50 mmol, 1.00 equiv). The reaction mixture was stirred at 25 °C for 1 hour. The mixture was extracted with EtOAc (20 mL×3) and washed with brine. The organic layer was dried over Na₂SO₄ and concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 68% B to 82% B in 12 min, Wavelength: 214 nm. This afforded the title compound (71 mg, 30%) as white solid. LCMS: [M+H]⁺=476.2. ¹H NMR (400 MHz, CDCl₃): δ 1.22-1.26 (2H, m), 1.35-1.43 (4H, m), 1.44-1.51 (4H, m), 1.58-1.64 (4H, m), 2.84 (3H, s), 2.89-2.97 (1H, m), 2.99 (3H, s), 3.48-3.54 (4H, m), 3.76-3.80 (2H, m), 3.85-3.87 (2H, m), 3.95-3.99 (2H, m), 5.39 (1H, s).

### Example 12. Synthesis of N-methyl-N-((1-(methylsulfonyl)piperidin-3-yl)methyl)-6-(2-azaspiro[5.5]undecan-2-yl)-2-(trifluoromethyl)pyrimidin-4-amine (Compound 14)

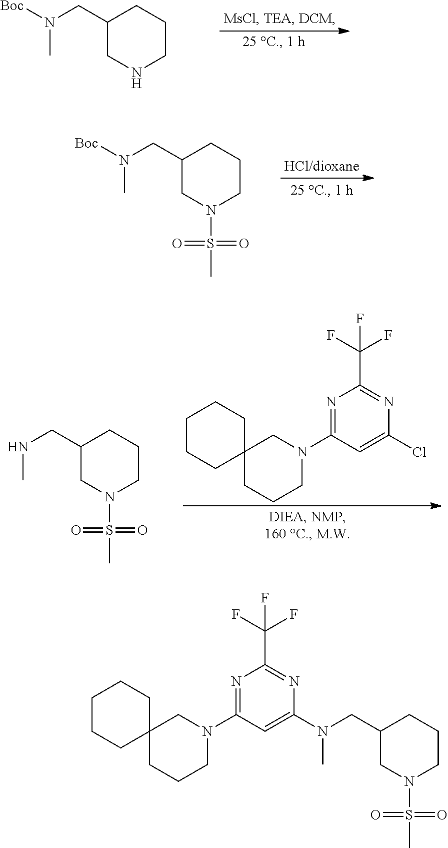 [SMILES: CN(CC1CCCNC1)C(=O)OC(C)(C)C ; CN(CC1CCCN(S(C)(=O)=O)C1)C(=O)OC(C)(C)C ; CNCC1CCCN(S(C)(=O)=O)C1 ; FC(F)(F)c1nc(Cl)cc(N2CCCC3(CCCCC3)C2)n1 ; CN(CC1CCCN(S(C)(=O)=O)C1)c1cc(N2CCCC3(CCCCC3)C2)nc(C(F)(F)F)n1]

### Step A. Synthesis of tert-butyl methyl((1-(methylsulfonyl)piperidin-3-yl)methyl)carbamate

To a solution of tert-butyl methyl(piperidin-3-ylmethyl)carbamate (228 mg, 1.00 mmol, 1.00 equiv) in DCM (15 mL) was added TEA (202 mg, 2.00 mmol, 2.00 equiv) and (137 mg, 1.20 mmol, 1.0 equiv) at 25 °C. The reaction mixture was stirred at 25 °C for 1 hour. Then the mixture was extracted with EtOAc (20 mL×3) and washed with brine. The organic layer was dried over Na₂SO₄ and concentrated in vacuo to give the tert-butyl methyl((1-(methylsulfonyl)piperidin-3-yl)methyl)carbamate crude (306 mg, 100%) as yellow oil which was used directly in the next step without further purification. LCMS: [M+H]⁺=307.

### Step B. Synthesis of N-methyl-1-(1-(methylsulfonyl)piperidin-3-yl)methanamine

A solution of tert-butyl methyl((1-(methylsulfonyl)piperidin-3-yl)methyl)carbamate (306 mg, 1.00 mmol, 1.00 equiv) in 4 M HCl/dioxane (6 mL) was stirred at 25 °C for 1 hour. Then the solution was removed in vacuo and the residue was dried in vacuo to get the N-methyl-1-(1-(methylsulfonyl)piperidin-3-yl)methanamine (20 mg, 100%) as yellow solid, which was used directly in the next step without further purification. LCMS: [M+H]⁺=207.

### Step C. Synthesis of N-methyl-N-((1-(methylsulfonyl)piperidin-3-yl)methyl)-6-(2-azaspiro[5.5]undecan-2-yl)-2-(trifluoromethyl)pyrimidin-4-amine

To a solution of 2-(6-chloro-2-(trifluoromethyl)pyrimidin-4-yl)-2-azaspiro[5.5]undecane (88 mg, 0.26 mmol, 1.00 equiv) in NMP (5 mL) were added DIEA (201 mg, 1.56 mmol, 6.00 equiv) and N-methyl-1-(1-(methylsulfonyl)piperidin-3-yl)methanamine (206 mg, 1.00 mmol, 3.85 equiv). The reaction mixture was stirred at 160 °C for 3 hours in a microwave. The mixture was extracted with EtOAc (20 mL×3) and washed with brine. The organic layer was dried over Na₂SO₄ and concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 80% B to 90% B in 11 min, Wavelength: 214 nm. This afforded the title compound (43 mg, 32%) as white solid. LCMS: [M+H]⁺=504.3. ¹H NMR (400 MHz, CDCl₃): δ 1.14-1.27 (4H, m), 1.37-1.40 (4H, m), 1.47-1.50 (5H, m), 1.58-1.67 (3H, m), 1.68-1.75 (1H, m), 1.81-1.86 (1H, m), 2.04-2.09 (1H, m), 2.54-2.60 (1H, m), 2.67-2.70 (1H, m), 2.72 (3H, s), 2.98 (3H, s), 3.35-3.40 (1H, m), 3.47-3.53 (5H, m), 3.62-3.65 (1H, m), 3.73-3.79 (1H, m), 5.39 (1H, s).

### Example 13. Synthesis of N-((4-(methylsulfonyl)morpholin-2-yl)methyl)-6-(2-azaspiro[5.5]undecan-2-yl)-2-(trifluoromethyl)pyrimidin-4-amine (Compound 15)

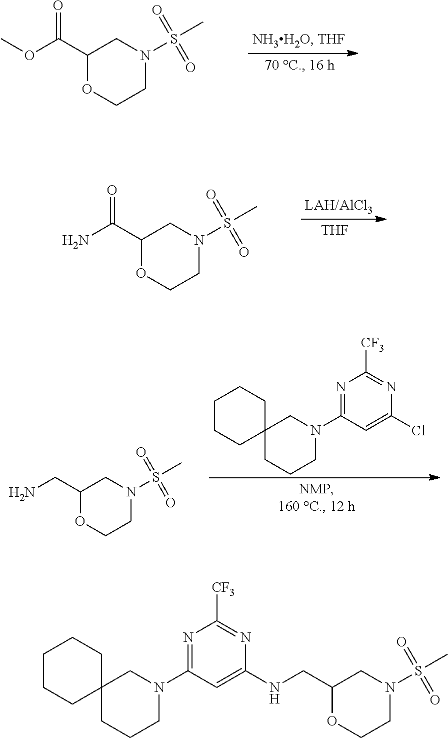

### Step A. Synthesis of 4-(methylsulfonyl)morpholine-2-carboxamide

To a solution of methyl 4-(methylsulfonyl)morpholine-2-carboxylate (300 mg, 1.35 mmol, 1.00 equiv) in THF/NH₃·H₂O (3 mL/6 mL). The mixture was stirred at 70 °C for 16 h under the sealed tube. The mixture was concentrated to get the 4-(methylsulfonyl)morpholine-2-carboxamide (277 mg, 98%) as white solid. LCMS: [M+H]⁺=209.

### Step B. Synthesis of (4-(methylsulfonyl)morpholin-2-yl)methanamine

To a solution of 4-(methylsulfonyl)morpholine-2-carboxamide (277 mg, 1.33 mmol, 1.00 equiv) in THF (48 mL) was added LiAlH₄ (4 mL, 1 M in THF, 3.00 equiv) and AlCl₃ (177 mg, 1.33 mmol, 1.00 equiv). The mixture was stirred at room temperature for 2 h. The mixture was diluted with ice/NaHCO₃ aqueous (10 mL), extracted with DCM (6×20 mL). The combined organic layers were washed with brine and concentrated in vacuo. This afforded (4-(methylsulfonyl)morpholin-2-yl)methanamine (195 mg, 94%) as white solid. LCMS: [M+H]⁺=195.

### Step C. Synthesis of N-((4-(methylsulfonyl)morpholin-2-yl)methyl)-6-(2-azaspiro[5.5]undecan-2-yl)-2-(trifluoromethyl)pyrimidin-4-amine

To a stirred solution of (4-(methylsulfonyl)morpholin-2-yl)methanamine (195 mg, 1.00 mmol, 4.10 equiv) and 2-(6-chloro-2-(trifluoromethyl)pyrimidin-4-yl)-2-azaspiro[5.5]undecane (80 mg, 0.24 mmol, 1.00 equiv) in NMP (5 mL) was added DIEA (93 mg, 0.72 mmol, 3.0 equiv). The resulting mixture was stirred at 160 °C for 12 h. The mixture was concentrated under reduced pressure. The resulting solution was purified using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 30% B to 60% B in 12 min; Wavelength: 214 nm. This afforded the title compound (50 mg, 42%) as a white solid. LCMS: [M+H]⁺=492. ¹H NMR (400 MHz, DMSO-d6): δ 1.16-1.30 (2H, m), 1.32-1.52 (12H, m), 2.59 (1H, m), 2.79-2.86 (1H, m), 2.88 (3H, s), 3.35-3.60 (10H, m), 3.92-3.95 (1H, m), 5.77 (1H, s), 7.15 (1H, t, J=2.8 Hz).

### Example 14. Synthesis of N-methyl-N-((4-(methylsulfonyl)morpholin-2-yl)methyl)-6-(6-oxa-9-azaspiro[4.5]decan-9-yl)-2-(trifluoromethyl)pyrimidin-4-amine (Compound 16)

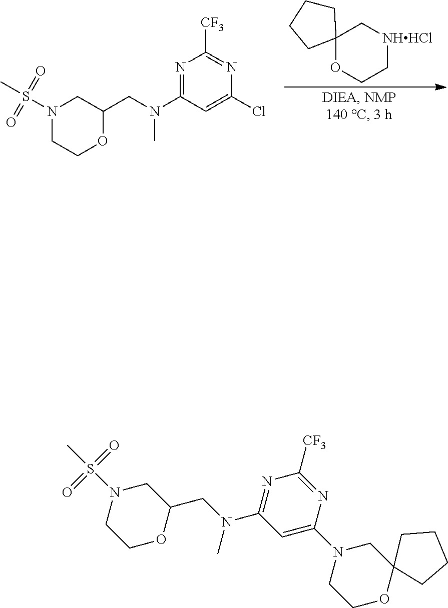

To a stirred solution of 6-chloro-N-methyl-N-((4-(methylsulfonyl)morpholin-2-yl)methyl)-2-(trifluoromethyl)pyrimidin-4-amine (117 mg, 0.30 mmol, 1.00 equiv) in NMP (3 mL) was added DIEA (116 mg, 0.90 mmol, 3.00 equiv) and 6-oxa-9-azaspiro[4.5]decane hydrochloride (80 mg, 0.45 mmol, 1.50 equiv). The resulting mixture was stirred at 140 °C for 3 hours. The reaction mixture was purified using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 65% B to 85% B in 12 min; Wavelength: 214 nm. This afforded the title compound (60 mg, 41%) as a white solid. LCMS: [M+H]⁺=494.1. ¹HNMR (400 MHz, DMSO-d₆): δ 1.55-1.67 (8H, m), 2.56-2.61 (1H, m), 2.79-2.82 (1H, m), 2.86 (3H, s), 3.05 (3H, s), 3.29-3.40 (2H, m), 3.46-3.73 (10H, m), 3.91-3.95 (1H, m), 5.85 (1H, s).

### Example 15. Synthesis of 6-(8-oxa-3-azabicyclo[3.2.1]octan-3-yl)-N-methyl-N-((4-(methylsulfonyl)morpholin-2-yl)methyl)-2-(trifluoromethyl)pyrimidin-4-amine (Compound 17)

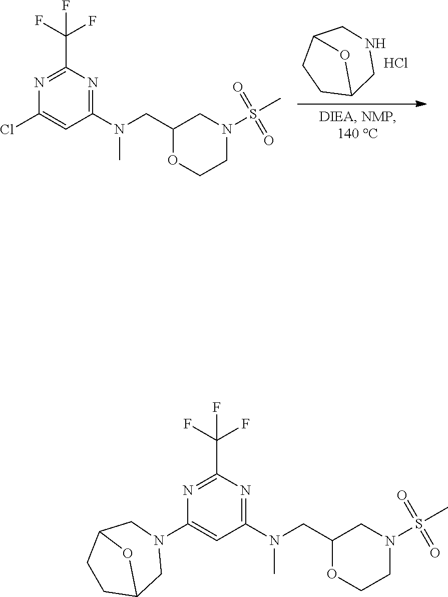

To a solution of 6-chloro-N-methyl-N-((4-(methylsulfonyl)morpholin-2-yl)methyl)-2-(trifluoromethyl)pyrimidin-4-amine (100 mg, 0.26 mmol, 1.00 equiv) and 8-oxa-3-azabicyclo[3.2.1]octane (47 mg, 0.31 mmol, 1.20 equiv) in NMP (3 mL) was added DIEA (101 mg, 0.78 mmol, 3.00 equiv). The mixture was stirred at 140 °C for 3 h. The mixture was extracted with EtOAc and washed with brine. The organic layer was dried over Na₂SO₄ and concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 40% B to 70% B in 9 min. Wavelength: 214 nm. This afforded the title compound (73 mg, 60%) as white solid. LCMS: [M+H]⁺=466.1. ¹H NMR (400 MHz, CDCl₃): δ 1.81-1.85 (2H, m), 1.94-1.99 (2H, m), 2.61-2.66 (1H, m), 2.78 (3H, s), 2.81-2.85 (1H, m), 3.04 (3H, s), 3.14-3.18 (2H, m), 3.41-3.46 (1H, m), 3.53-3.56 (2H, m), 3.63-3.69 (1H, m), 3.73-3.93 (3H, m), 3.98-4.10 (2H, m), 4.74 (2H, s), 5.37 (1H, s).

### Example 16. Synthesis of 6-(6-oxa-3-azabicyclo[3.1.1]heptan-3-yl)-N-methyl-N-((4-(methylsulfonyl)morpholin-2-yl)methyl)-2-(trifluoromethyl)pyrimidin-4-amine (Compound 18)

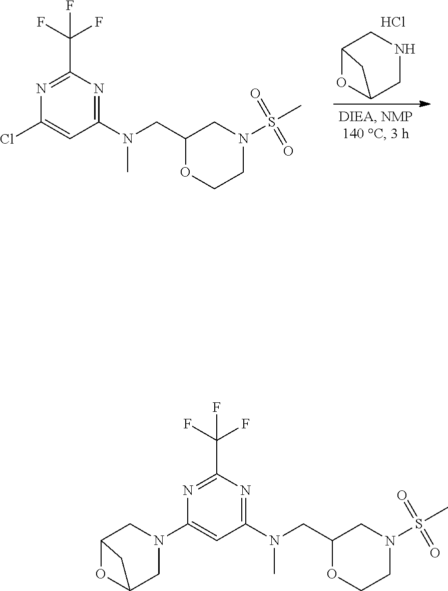 [SMILES: CN(CC1CN(S(C)(=O)=O)CCO1)c1cc(Cl)nc(C(F)(F)F)n1 ; C1NCC2CC1O2 ; Cl ; CN(CC1CN(S(C)(=O)=O)CCO1)c1cc(N2CC3CC(C2)O3)nc(C(F)(F)F)n1]

To a solution of 6-chloro-N-methyl-N-((4-(methylsulfonyl)morpholin-2-yl)methyl)-2-(trifluoromethyl)pyrimidin-4-amine (110 mg, 0.28 mmol, 1.00 equiv) in NMP (5 mL) was added DIEA (73 mg, 0.57 mmol, 2.00 equiv) and 6-oxa-3-azabicyclo[3.1.1]heptane (42 mg, 0.31 mmol, 1.10 equiv) at r.t. The mixture was stirred at 140 °C for 3 h. The mixture was extracted with EA (10.00 mL×3) and water (10.00 mL). The organic layer was dried over Na₂SO₄ and concentrated in vacuum to remove the solvent. The residue was purified by Prep-HPLC with the following conditions: Column: XBridge C18 SN271/3611211208 Waters, 19×150 mm, 5 μm; Mobile Phase A: Water (0.1% NH₄HCO₃), Mobile Phase B: ACN; Flow rate: 15 mL/min; Gradient: 45% B to 55% B in 12 min. Wavelength: 214 nm. This afforded the title compound (46 mg, 36%) as a white solid. LCMS: [M+H]⁺=452.1. ¹H NMR (400 MHz, CDCl₃): δ 1.94 (1H, d, J=8.8 Hz), 2.60-2.68 (1H, m), 2.75 (3H, s), 2.77-2.86 (1H, m), 3.07 (3H, s), 3.25-3.33 (1H, m), 3.43-3.83 (9H, m), 3.96-4.03 (1H, m), 4.04-4.14 (1H, m), 4.74 (2H, d, J=6.4 Hz), 5.39 (1H, s)

### Example 17. Synthesis of N-benzyl-N-((4-(methylsulfonyl)morpholin-2-yl)methyl)-6-(1-oxa-4-azaspiro[5.5]undecan-4-yl)-2-(trifluoromethyl)pyrimidin-4-amine (Compound 19)

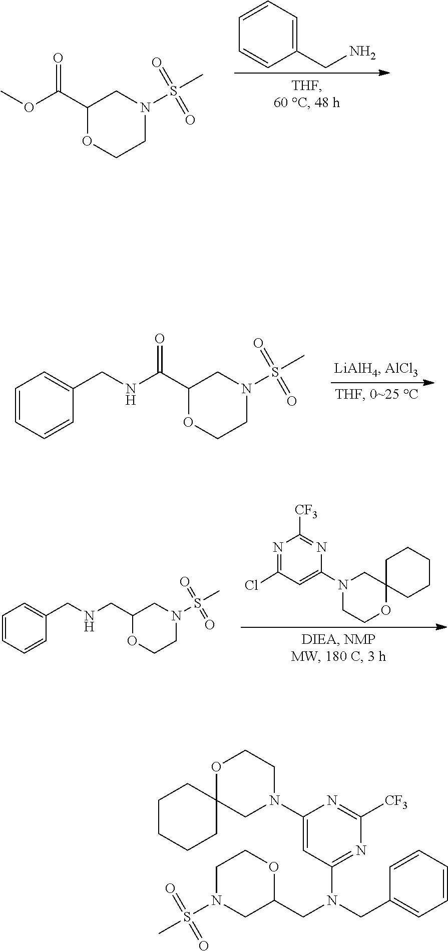

### Step A. Synthesis of N-benzyl-4-(methylsulfonyl)morpholine-2-carboxamide

To a solution of methyl 4-(methylsulfonyl)morpholine-2-carboxylate (250 mg, 1.12 mmol, 1.00 equiv.) in THF (5 mL) added phenylmethanamine (360 mg, 3.36 mmol, 3.00 equiv.). The mixture was stirred at 60 °C for 48 h. The mixture was concentrated to get crude product as yellow oil and the residue crude was washed with PE to get the N-benzyl-4-(methylsulfonyl)morpholine-2-carboxamide (364 mg, >100%) as light yellow solid. LCMS: [M+H]⁺=299.

### Step B. Synthesis of N-benzyl-1-(4-(methylsulfonyl)morpholin-2-yl)methanamine

To a solution of AlCl₃ (118 mg, 0.88 mmol, 1.00 equiv.) in THF (5 mL) at 0 °C was added LiAlH₄/THF (2.6 mL, 2.65 mmol, 3.00 equiv.). The mixture was stirred at 25 °C for 15 min and then added N-benzyl-4-(methylsulfonyl)morpholine-2-carboxamide (264 mg, 0.88 mmol, 1.00 equiv.) at 0 °C. The mixture was stirred at 25 °C for 6 h. The mixture was added to saturated Na₂SO₄ solutions at 0 °C, diluted with EA, filtered and dried over Na₂SO₄. The mixture was concentrated and purified by Flash chromatography (DCM/MeOH=20/1~10/1) to get the N-benzyl-1-(4-(methylsulfonyl)morpholin-2-yl)methanamine (110 mg, 44%) as yellow oil. LCMS: [M+H]⁺=285.

### Step C. Synthesis of N-benzyl-N-((4-(methylsulfonyl)morpholin-2-yl)methyl)-6-(1-oxa-4-azaspiro[5.5]undecan-4-yl)-2-(trifluoromethyl)pyrimidin-4-amine

To a solution of N-benzyl-1-(4-(methylsulfonyl)morpholin-2-yl)methanamine (90 mg, 0.32 mmol, 1.00 equiv.) in NMP (2 mL) was added 4-(6-chloro-2-(trifluoromethyl)pyrimidin-4-yl)-1-oxa-4-azaspiro[5.5]undecane (107 mg, 0.32 mmol, 1.00 equiv.) and DIEA (106 mg, 0.79 mmol, 2.50 equiv.). The mixture was irradiated in a microwave at 180 °C for 3 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 55% B to 90% B in 10 min. Wavelength: 214 nm. This afforded the title compound (38 mg, 20%) as white solid. LCMS: [M+H]⁺=584.2. ¹H NMR (400 MHz, DMSO-d₆): δ 1.20-1.69 (11H, m), 2.64-2.73 (1H, m), 2.78-2.89 (1H, m), 2.92 (3H, s), 3.42-3.70 (9H, m), 3.75-3.83 (2H, m), 3.98-4.02 (1H, m), 4.73-4.79 (1H, m), 4.98-5.02 (1H, m), 5.90 (1H, s), 7.25-7.35 (3H, m), 7.35-7.45 (2H, m).

### Example 18. Synthesis of N-ethyl-N-((4-(methylsulfonyl)morpholin-2-yl)methyl)-6-(1-oxa-4-azaspiro[5.5]undecan-4-yl)-2-(trifluoromethyl)pyrimidin-4-amine (Compound 20)

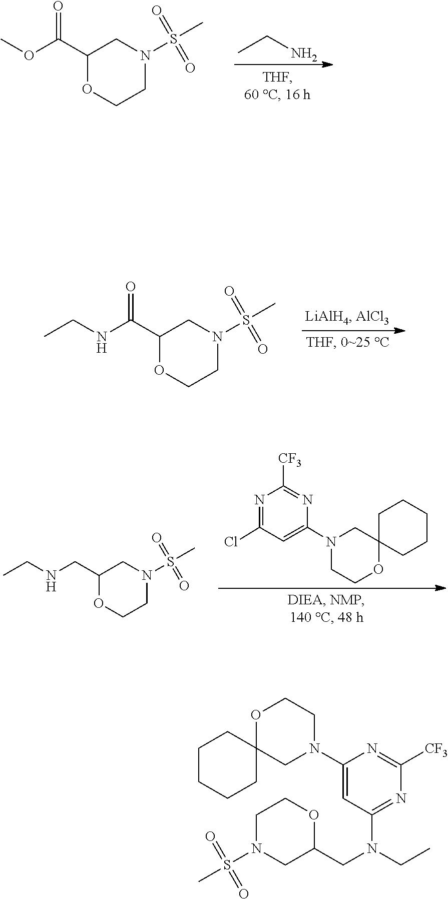

### Step A. Synthesis of N-ethyl-4-(methylsulfonyl)morpholine-2-carboxamide

To methyl 4-(methylsulfonyl)morpholine-2-carboxylate (200 mg, 0.90 mmol, 1.00 equiv.) was added ethanamine (5 mL, 1M in THF). The mixture was stirred at 60 °C in a sealed tube for 16 h. The mixture was concentrated to get crude N-ethyl-4-(methylsulfonyl)morpholine-2-carboxamide (200 mg, 95%) as light yellow solid. LCMS: [M+H]⁺=237.

### Step B. Synthesis of N-((4-(methylsulfonyl)morpholin-2-yl)methyl)ethanamine

To a solution of AlCl₃ (113 mg, 0.85 mmol, 1.00 equiv.) in THF (5 mL) was added LiAlH₄ (2.6 mL, 2.54 mmol, 3.00 equiv.) at 0 °C. The mixture was stirred at 25 °C for 15 min and then added N-ethyl-4-(methylsulfonyl)morpholine-2-carboxamide (200 mg, 0.85 mmol, 1.00 equiv.) at 0 °C. The mixture was stirred at 25 °C for 24 h. The mixture was added to saturated Na₂SO₄ solution at 0 °C, diluted with EA, filtered and dried over Na₂SO₄. The mixture was filtered, concentrated and purified by Flash chromatography (DCM/MeOH=20/1~10/1) to get the N-((4-(methylsulfonyl)morpholin-2-yl)methyl)ethanamine (200 mg, >100%) as yellow oil. LCMS: [M+H]⁺=223

### Step C. Synthesis of N-ethyl-N-((4-(methylsulfonyl)morpholin-2-yl)methyl)-6-(1-oxa-4-azaspiro[5.5]undecan-4-yl)-2-(trifluoromethyl)pyrimidin-4-amine (ANM-0005608)

To a solution of N-((4-(methylsulfonyl)morpholin-2-yl)methyl)ethanamine (100 mg, 0.45 mmol, 1.00 equiv.) in NMP (2 mL) was added 4-(6-chloro-2-(trifluoromethyl)pyrimidin-4-yl)-1-oxa-4-azaspiro[5.5]undecane (151 mg, 0.45 mmol, 1.00 equiv.) and DIEA (144 mg, 1.12 mmol, 2.50 equiv.). The mixture was stirred at 140 °C for 48 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 50% B to 90% B in 10 min. Wavelength: 214 nm. This afforded the title compound (40 mg, 17%) as white solid. LCMS: [M+H]⁺=522.2. ¹H NMR (400 MHz, DMSO-d6): δ 1.08 (3H, t, J=7.2 Hz), 1.20-1.48 (9H, m), 1.58-1.65 (2H, m), 2.57-2.63 (1H, m), 2.78-2.83 (1H, m), 2.86 (3H, s), 3.40-3.57 (9H, m), 3.64-3.70 (4H, m), 3.91-3.95 (1H, m), 5.84 (1H, s).

### Example 19. Synthesis of 6-(9,9-difluoro-1-oxa-4-azaspiro[5.5]undecan-4-yl)-N-methyl-N-((4-(methylsulfonyl)morpholin-2-yl)methyl)-2-(trifluoromethyl)pyrimidin-4-amine (Compound 21)

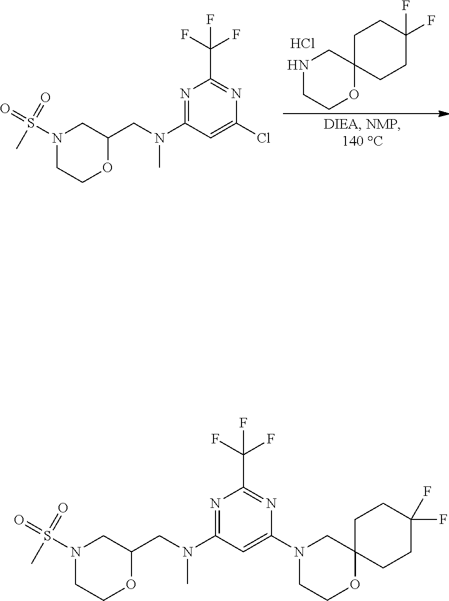 [SMILES: CN(CC1CN(S(C)(=O)=O)CCO1)c1cc(Cl)nc(C(F)(F)F)n1 ; Cl ; FC1(F)CCC2(CC1)CNCCO2 ; CN(CC1CN(S(C)(=O)=O)CCO1)c1cc(N2CCOC3(CCC(F)(F)CC3)C2)nc(C(F)(F)F)n1]

To a solution of 6-chloro-N-methyl-N-((4-(methylsulfonyl)morpholin-2-yl)methyl)-2-(trifluoromethyl)pyrimidin-4-amine (80 mg, 0.20 mmol, 1.00 equiv) and DIEA (52 mg, 0.30 mmol, 2.00 equiv) in NMP (2 mL) was added 9,9-difluoro-1-oxa-4-azaspiro[5.5]undecane (HCl salt) (68 mg, 0.30 mmol, 1.50 equiv). The mixture was stirred at 140 °C for 4 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 40% B to 90% B in 9 min, Wavelength: 214 nm. This afforded the title compound (69 mg, 63%) as white solid. LCMS: [M+H]⁺=544.2. ¹H NMR (400 MHz, CDCl₃): δ 1.61 (2H, s), 1.90-2.06 (6H, m), 2.58-2.67 (1H, m), 2.74 (3H, s), 2.76-2.85 (1H, m), 3.04 (3H, s), 3.40-3.44 (1H, m), 3.47-3.55 (4H, m), 3.56-3.70 (3H, m), 3.74-3.79 (3H, m), 3.95-4.00 (1H, m), 4.01-4.02 (1H, m), 5.40 (1H, s).

### Example 20. Synthesis of N-((4,4-difluoro-1-(methylsulfonyl)piperidin-3-yl)methyl)-6-(5,5-difluoro-2-azaspiro[5.5]undecan-2-yl)-N-methyl-2-(trifluoromethyl)pyrimidin-4-amine (Compound 22)

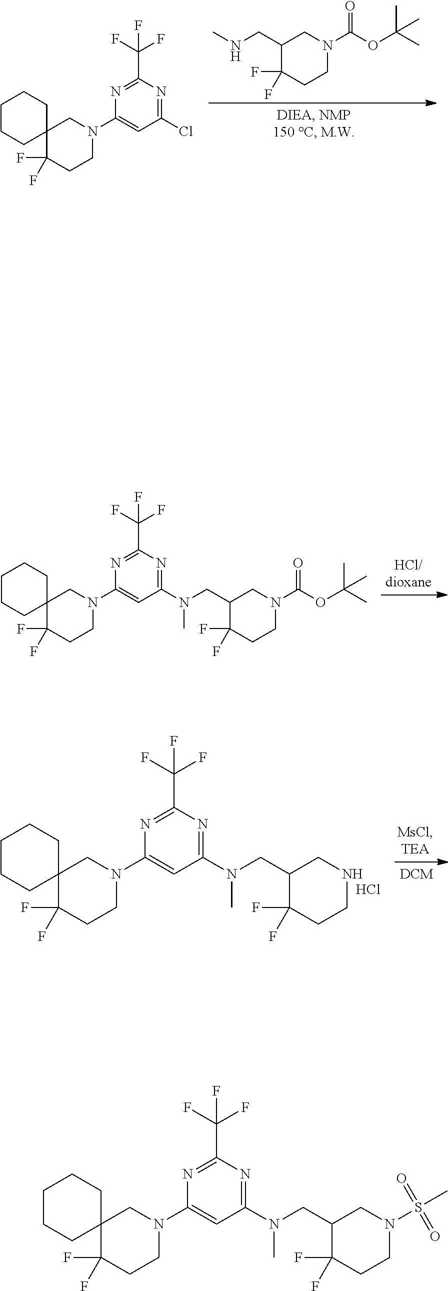 [SMILES: FC(F)(F)c1nc(Cl)cc(N2CCC(F)(F)C3(CCCCC3)C2)n1 ; CNCC1CN(C(=O)OC(C)(C)C)CCC1(F)F ; CN(CC1CN(C(=O)OC(C)(C)C)CCC1(F)F)c1cc(N2CCC(F)(F)C3(CCCCC3)C2)nc(C(F)(F)F)n1 ; CN(CC1CNCCC1(F)F)c1cc(N2CCC(F)(F)C3(CCCCC3)C2)nc(C(F)(F)F)n1 ; Cl ; CN(CC1CN(S(C)(=O)=O)CCC1(F)F)c1cc(N2CCC(F)(F)C3(CCCCC3)C2)nc(C(F)(F)F)n1]

### Step A. Synthesis of tert-butyl 3-(((6-(5,5-difluoro-2-azaspiro[5.5]undecan-2-yl)-2-(trifluoromethyl)pyrimidin-4-yl)(methyl)amino)methyl)-4,4-difluoropiperidine-1-carboxylate

To a solution of 2-(6-chloro-2-(trifluoromethyl)pyrimidin-4-yl)-5,5-difluoro-2-azaspiro[5.5]undecane (70 mg, 0.19 mmol, 1.00 equiv) and tert-butyl 4,4-difluoro-3-((methylamino)methyl)piperidine-1-carboxylate (75 mg, 0.28 mmol, 1.50 equiv) in NMP (2 mL) was added DIEA (49 mg, 0.38 mmol, 2.00 equiv). The mixture was stirred at 150 °C for 2 h in microwave. The mixture was extracted with EtOAc and washed with brine. The organic layer was dried over Na₂SO₄ and concentrated by vacuo to get tert-butyl 3-(((6-(5,5-difluoro-2-azaspiro[5.5]undecan-2-yl)-2-(trifluoromethyl)pyrimidin-4-yl)(methyl)amino)methyl)-4,4-difluoropiperidine-1-carboxylate (56 mg, 49%) as yellow solid. LCMS: [M+H]⁺=598.

### Step B. Synthesis of 6-(5,5-difluoro-2-azaspiro[5.5]undecan-2-yl)-N-((4,4-difluoropiperidin-3-yl)methyl)-N-methyl-2-(trifluoromethyl)pyrimidin-4-amine (HCl Salt)

To a solution of tert-butyl 3-(((6-(5,5-difluoro-2-azaspiro[5.5]undecan-2-yl)-2-(trifluoromethyl)pyrimidin-4-yl)(methyl)amino)methyl)-4,4-difluoropiperidine-1-carboxylate (56 mg, 0.09 mmol, 1.00 equiv in DCM (1 mL) was added 4M HCl/dioxane (0.5 mL). The mixture was stirred at room temperature for 2 h. The mixture was concentrated to get the crude 6-(5,5-difluoro-2-azaspiro[5.5]undecan-2-yl)-N-((4,4-difluoropiperidin-3-yl)methyl)-N-methyl-2-(trifluoromethyl)pyrimidin-4-amine (HCl salt) (57 mg, >100%) as yellow solid. LCMS: [M+H]⁺=498.

### Step C. Synthesis of N-((4,4-difluoro-1-(methylsulfonyl)piperidin-3-yl)methyl)-6-(5,5-difluoro-2-azaspiro[5.5]undecan-2-yl)-N-methyl-2-(trifluoromethyl)pyrimidin-4-amine

To a solution of 6-(5,5-difluoro-2-azaspiro[5.5]undecan-2-yl)-N-((4,4-difluoropiperidin-3-yl)methyl)-N-methyl-2-(trifluoromethyl)pyrimidin-4-amine (HCl salt) (57 mg, 0.11 mmol, 1.00 equiv) and TEA (23 mg, 0.22 mmol, 2.00 equiv) in DCM (2 mL) was added MsCl (19 mg, 0.17 mmol, 1.50 equiv). The mixture was stirred at room temperature for 1 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 75% B to 90% B in 10 min, Wavelength: 214 nm. This afforded the title compound (10 mg, 15%) as white solid. LCMS: [M+H]⁺=576.2. ¹H NMR (400 MHz, CDCl₃): δ 1.18-1.19 (2H, m), 1.45-1.52 (5H, m), 1.60-1.75 (3H, m), 1.94-2.10 (4H, m), 2.15-2.25 (1H, m), 2.79 (3H, s), 2.94-2.99 (1H, m), 3.03 (3H, s), 3.07-3.11 (1H, m), 3.50-3.56 (1H, m), 3.57-3.80 (6H, m), 4.00-4.12 (1H, m), 5.49 (1H, s).

### Example 21. Synthesis of 6-(5,5-difluoro-2-azaspiro[5.5]undecan-2-yl)-N-methyl-N-((4-(methylsulfonyl)morpholin-2-yl)methyl)-2-(trifluoromethyl)pyrimidin-4-amine (Compound 23)

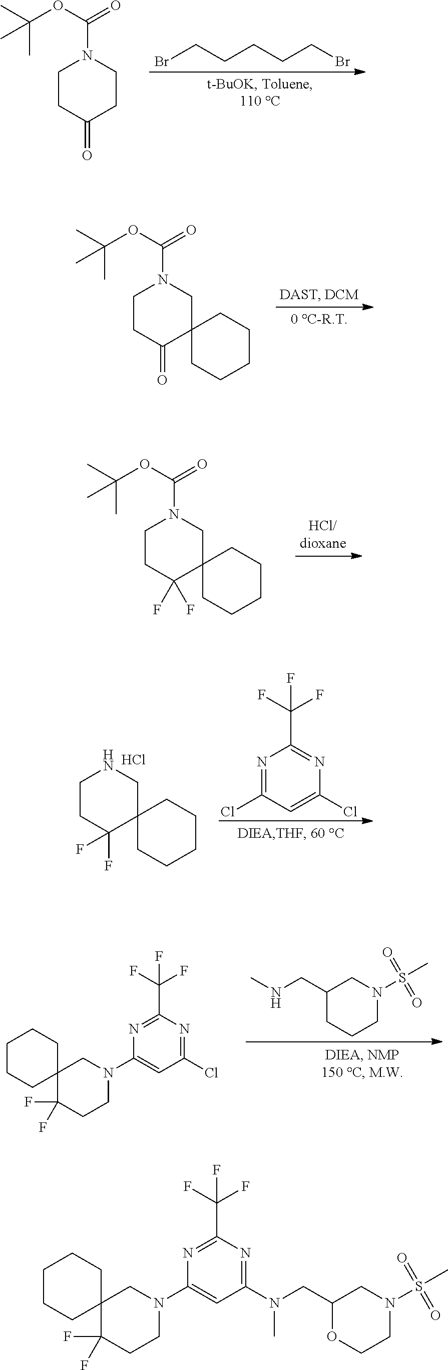

### Step A. Synthesis of tert-butyl 5-oxo-2-azaspiro[5.5]undecane-2-carboxylate

To a solution of tert-butyl 4-oxopiperidine-1-carboxylate (10.0 g, 0.05 mol, 1.00 equiv) in Toluene (80 mL) was added t-BuOK (14.0 g, 0.125 mol, 2.50 equiv) under N₂ at rt. After 1 h 1,5-dibromopentane (11.5 g, 0.05 mol, 1.00 equiv) was added dropwise over 10 min and the reaction heated at reflux for 3 h. The mixture was extracted with EtOAc and washed with saturated NH₄Cl. The organic layer was dried over Na₂SO₄ and concentrated. The residue was purified by column eluted with PE/EA=10/1 to get the tert-butyl 5-oxo-2-azaspiro[5.5]undecane-2-carboxylate (629 mg, 4.0%) as yellow oil. LCMS: [M+H-56]⁺=212.

### Step B. Synthesis of tert-butyl 5,5-difluoro-2-azaspiro[5.5]undecane-2-carboxylate

To a solution of tert-butyl 5-oxo-2-azaspiro[5.5]undecane-2-carboxylate (280 mg, 1.04 mmol, 1.00 equiv) in DCM (5 mL) at 0 °C was added DAST (252 mg, 1.50 mmol, 1.50 equiv). The mixture was stirred at room temperature for 18 h. The mixture was added 20 mL saturated NaHCO₃ and extracted with DCM and washed with brine. The organic layer was dried over Na₂SO₄ and concentrated by vacuo to get the crude tert-butyl 5,5-difluoro-2-azaspiro[5.5]undecane-2-carboxylate (312 mg, >100%) as yellow oil. LCMS: [M+H]⁺=290.

### Step C. Synthesis of 5,5-difluoro-2-azaspiro[5.5]undecane

To a solution of tert-butyl 5,5-difluoro-2-azaspiro[5.5]undecane-2-carboxylate (312 mg, 1.07 mmol, 1.00 equiv) in DCM (1 mL) was added 2 mL HCl (4 N in 1,4-dioxane). The mixture was stirred at room temperature for 2 h. The mixture was concentrated to get the crude 5,5-difluoro-2-azaspiro[5.5]undecane (HCl salt) (300 mg, >100%) as yellow oil. LCMS: [M+H]⁺=190.

### Step D. Synthesis of 2-(6-chloro-2-(trifluoromethyl)pyrimidin-4-yl)-5,5-difluoro-2-azaspiro[5.5]undecane

To a solution of 4,6-dichloro-2-(trifluoromethyl)py (397 mg, 1.80 mmol, 1.00 equiv in THF (10 mL) was added 5,5-difluoro-2-azaspiro[5.5]undecane (HCl salt) (350 mg, 1.80 mmol, 1.00 equiv) and DIEA (468 mg, 3.6 mmol, 2.00 equiv). The mixture was stirred at 60 °C for 4 h. The mixture was concentrated and purified by column eluted with PE/EA=10/1 to get the 2-(6-chloro-2-(trifluoromethyl)pyrimidin-4-yl)-5,5-difluoro-2-azaspiro[5.5]undecane (178 mg, 26%) as yellow oil. LCMS: [M+H]⁺=370.

### Step E. Synthesis of 6-(5,5-difluoro-2-azaspiro[5.5]undecan-2-yl)-N-methyl-N-((4-(methylsulfonyl)morpholin-2-yl)methyl)-2-(trifluoromethyl)pyrimidin-4-amine

To a solution of 2-(6-chloro-2-(trifluoromethyl)pyrimidin-4-yl)-5,5-difluoro-2-azaspiro[5.5]undecane (50 mg, 0.13 mmol, 1.00 equiv) and DIEA (42 mg, 0.33 mmol, 2.50 equiv) in NMP (2 mL) was added N-methyl-1-(4-(methylsulfonyl)morpholin-2-yl)methanamine (HCl salt) (65 mg, 0.27 mmol, 2.00 equiv). The mixture was stirred at 150 °C for 2 h in a microwave. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 70% B to 85% B in 8 min, Wavelength: 214 nm. This afforded the title compound (30 mg, 43%) as white solid. LCMS: [M+H]⁺=542.2. ¹H NMR (400 MHz, CDCl₃): δ 1.12-1.30 (1H, m), 1.40-1.55 (6H, m), 1.57-1.65 (3H, m), 1.95-2.10 (2H, m), 2.60-2.67 (1H, m), 2.75 (3H, s), 2.76-2.85 (1H, m), 3.05 (3H, s), 3.40-3.46 (1H, m), 3.52-3.59 (2H, m), 3.62-3.67 (3H, m), 3.66-3.79 (3H, m), 3.79-4.09 (2H, m), 5.48 (1H, s).

### Example 22. Synthesis of N-((4,4-difluoro-1-(methylsulfonyl)piperidin-3-yl)methyl)-N-methyl-6-(1-oxa-4-azaspiro[5.5]undecan-4-yl)-2-(trifluoromethyl)pyrimidin-4-amine (Compound 24)

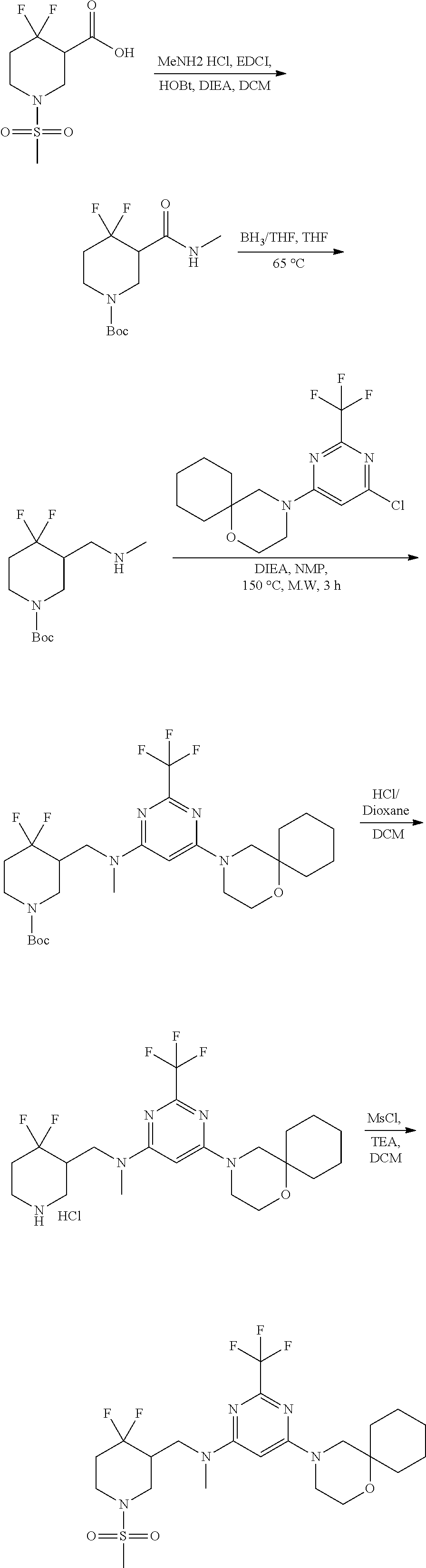

### Step A. Synthesis of tert-butyl 4,4-difluoro-3-(methylcarbamoyl)piperidine-1-carboxylate

To a stirred solution of 1-(tert-butoxycarbonyl)-4,4-difluoropiperidine-3-carboxylic acid (255 mg, 0.96 mmol, 1.00 equiv) in DCM (8 mL) was added DIEA (372 mg, 0.2.88 mmol, 3.00 equiv), EDCI (277 mg, 1.44 mmol, 1.50 equiv), HOBt (130 mg, 0.96 mmol, 1.00 equiv) and Methylamine hydrochloride (97 mg, 1.44 mmol, 1.50 equiv). The resulting mixture was stirred at R.T. for 16 h. The mixture was added water (20 mL), extracted with DCM (10 mL). The combined organic layer was washed with brine and concentrated in vacuo. The residue was purified by flash chromatography (EA/PE=33%~50%) to afford tert-butyl 4,4-difluoro-3-(methylcarbamoyl)piperidine-1-carboxylate (230 mg, 86%) as colorless oil. LCMS: [M+H-56]=223.

### Step B. Synthesis of tert-butyl 4,4-difluoro-3-((methylamino)methyl)piperidine-1-carboxylate

To a stirred solution of tert-butyl 4,4-difluoro-3-(methylcarbamoyl)piperidine-1-carboxylate (230 mg, 0.83 mmol, 1.00 equiv) in THF (6 mL) was added dropwise 1.0 M Borane-tetrahydrofuran complex (8.3 mL, 8.27 mmol, 10.00 equiv). The resulting mixture was stirred at 65 °C for 3 h. The mixture was cooled to R.T. and quenched with MeOH (5 mL) slowly, stirred at R.T. for 30 min. The mixture was concentrated in vacuo to give the crude tert-butyl 4,4-difluoro-3-((methylamino)methyl)piperidine-1-carboxylate (295 mg, >100%) as colorless oil. LCMS: [M+H]=265.

### Step C. Synthesis of tert-butyl 3-(((6-(1-oxa-4-azaspiro[5.5]undecan-4-yl)-2-(trifluoromethyl)pyrimidin-4-yl)(methyl)amino)methyl)-4,4-difluoropiperidine-1-carboxylate

To a stirred solution of 4-(6-chloro-2-(trifluoromethyl)pyrimidin-4-yl)-1-oxa-4-azaspiro[5.5]undecane (88 mg, 0.26 mmol, 1.00 equiv) in NMP (3 mL) was added DIEA (101 mg, 0.78 mmol, 3.00 equiv) and tert-butyl 4,4-difluoro-3-((methylamino)methyl)piperidine-1-carboxylate (crude 100 mg, 0.37 mmol, 1.45 equiv). The resulting mixture was stirred at 150 °C for 3 h in a microwave. To the mixture was added water (20 mL), extracted with EtOAc (10 mL). The combined organic layer was washed with brine and concentrated in vacuo. The residue was purified by flash chromatography (EA/PE=6%) to afford tert-butyl 3-(((6-(1-oxa-4-azaspiro[5.5]undecan-4-yl)-2-(trifluoromethyl)pyrimidin-4-yl)(methyl)amino)methyl)-4,4-difluoropiperidine-1-carboxylate (102 mg, 69%) as a yellow oil. LCMS: [M+H]=564

### Step D. Synthesis of N-((4,4-difluoropiperidin-3-yl)methyl)-N-methyl-6-(1-oxa-4-azaspiro[5.5]undecan-4-yl)-2-(trifluoromethyl)pyrimidin-4-amine hydrochloride

To a solution of tert-butyl 3-(((6-(1-oxa-4-azaspiro[5.5]undecan-4-yl)-2-(trifluoromethyl)pyrimidin-4-yl)(methyl)amino)methyl)-4,4-difluoropiperidine-1-carboxylate (77 mg, 0.14 mmol, 1.00 equiv) in DCM (4 mL) was added 4M HCl/dioxane (1 mL). The mixture was stirred at R.T. for 1 h. The mixture was concentrated in vacuo to get the crude N-((4,4-difluoropiperidin-3-yl)methyl)-N-methyl-6-(1-oxa-4-azaspiro[5.5]undecan-4-yl)-2-(trifluoromethyl)pyrimidin-4-amine hydrochloride (110 mg, >100%) as yellow oil. LCMS: [M+H]⁺=464.

### Step E. Synthesis of N-((4,4-difluoro-1-(methylsulfonyl)piperidin-3-yl)methyl)-N-methyl-6-(1-oxa-4-azaspiro[5.5]undecan-4-yl)-2-(trifluoromethyl)pyrimidin-4-amine (ANM-0005613)

To a solution of N-((4,4-difluoropiperidin-3-yl)methyl)-N-methyl-6-(1-oxa-4-azaspiro[5.5]undecan-4-yl)-2-(trifluoromethyl)pyrimidin-4-amine hydrochloride (crude 110 mg, 0.14 mmol, 1.00 equiv) in DCM (4 mL) was added TEA (42 mg, 0.41 mmol, 3.00 equiv) and MsCl (24 mg, 0.26 mmol, 1.50 equiv). The mixture was stirred at R.T. for 0.5 h. The mixture was added water, extracted with DCM. The combined organics was concentrated in vacuo. The residue was purified using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 50% B to 90% B in 12 min; Wavelength: 214 nm. This afforded the title compound (25 mg, 33%) as a white solid. LCMS: [M+H]⁺=542.2. ¹HNMR (400 MHz, CDCl₃-d): δ 1.25-1.64 (9H, m), 1.72-1.75 (2H, m), 2.03-2.23 (2H, m), 2.41-2.48 (1H, m), 2.78 (3H, s), 2.95-3.09 (5H, m), 3.46-3.79 (9H, m), 4.12-4.17 (1H, m), 5.34 (1H, s).

### Example 23. Synthesis of 2-(((6-(1-oxa-4-azaspiro[5.5]undecan-4-yl)-2-(trifluoromethyl)pyrimidin-4-yl)(methyl)amino)methyl)-4-(methylsulfonyl)thiomorpholine 1,1-dioxide (Compound 25)

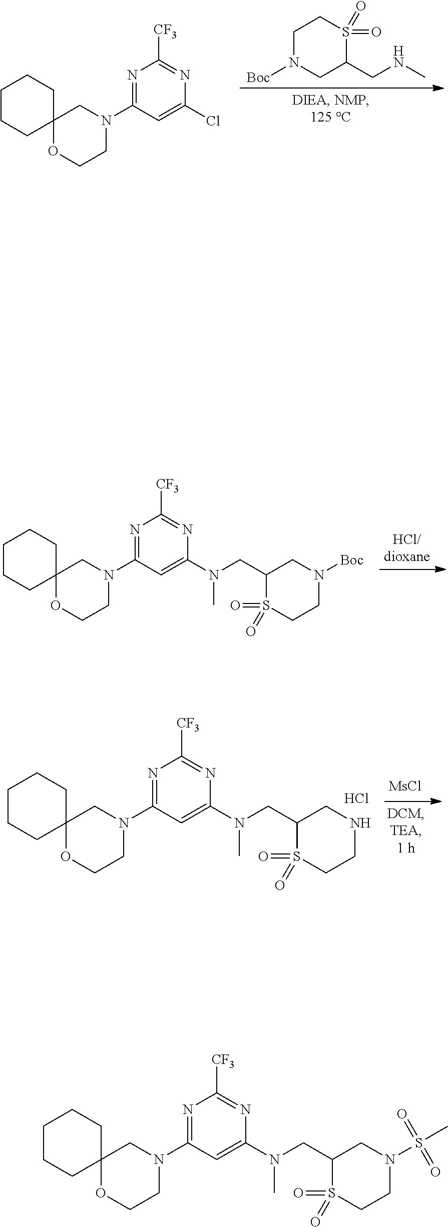

### Step A. Synthesis of tert-butyl 2-(((6-(1-oxa-4-azaspiro[5.5]undecan-4-yl)-2-(trifluoromethyl)pyrimidin-4-yl)(methyl)amino)methyl)thiomorpholine-4-carboxylate 1,1-dioxide

To a solution of 4-(6-chloro-2-(trifluoromethyl)pyrimidin-4-yl)-1-oxa-4-azaspiro[5.5]undecane (50 mg, 0.15 mmol, 1.00 equiv) and tert-butyl 2-((methylamino)methyl)thiomorpholine-4-carboxylate 1,1-dioxide (83 mg, 0.30 mmol, 2.00 equiv) in NMP (2 mL) was added DIEA (58 mg, 0.45 mmol, 3.00 equiv). The mixture was stirred at 125 °C for 16 h. The mixture was extracted with EtOAc and washed with brine. The organic layer was dried over Na₂SO₄ and concentrated. The residue was purified by column eluted with PE/EA=5/1 to get the tert-butyl 2-(((6-(1-oxa-4-azaspiro[5.5]undecan-4-yl)-2-(trifluoromethyl)pyrimidin-4-yl)(methyl)amino)methyl)thiomorpholine-4-carboxylate 1,1-dioxide (47 mg, 66%) as yellow oil. LCMS: [M+H]⁺=578.

### Step B. Synthesis of 2-(((6-(1-oxa-4-azaspiro[5.5]undecan-4-yl)-2-(trifluoromethyl)pyrimidin-4-yl)(methyl)amino)methyl)thiomorpholine 1,1-dioxide (HCl Salt)

To a solution of tert-butyl 2-(((6-(1-oxa-4-azaspiro[5.5]undecan-4-yl)-2-(trifluoromethyl)pyrimidin-4-yl)(methyl)amino)methyl)thiomorpholine-4-carboxylate 1,1-dioxide (47 mg, 0.08 mmol, 1.00 equiv) in DCM (3 mL) was added 1 mL HCl (4N in 1,4-dioxane). The mixture was stirred at room temperature for 2 h. The mixture was concentrated to get the crude 2-(((6-(1-oxa-4-azaspiro[5.5]undecan-4-yl)-2-(trifluoromethyl)pyrimidin-4-yl)(methyl)amino)methyl)thiomorpholine 1,1-dioxide (HCl salt) (51 mg, >100%) as yellow oil. LCMS: [M+H]⁺=478.

### Step C. Synthesis of 2-(((6-(1-oxa-4-azaspiro[5.5]undecan-4-yl)-2-(trifluoromethyl)pyrimidin-4-yl)(methyl)amino)methyl)-4-(methylsulfonyl)thiomorpholine 1,1-dioxide

To a solution of crude 2-(((6-(1-oxa-4-azaspiro[5.5]undecan-4-yl)-2-(trifluoromethyl)pyrimidin-4-yl)(methyl)amino)methyl)thiomorpholine 1,1-dioxide (HCl salt) (50 mg, 0.10 mmol, 1.00 equiv) and TEA (29 mg, 0.29 mmol, 3.00 equiv) in DCM (3 mL) was added MsCl (22 mg, 0.20 mmol, 2.00 equiv). The mixture was stirred at room temperature for 1 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 55% B to 90% B in 10 min. Wavelength: 214 nm. This afforded the title compound (21 mg, 39%) as white solid. LCMS: [M+H]⁺=556.2. ¹H NMR (400 MHz, CD₃OD): δ 1.31-1.74 (11H, m), 2.96 (3H, s), 3.14 (3H, s), 3.27-3.29 (1H, m), 3.49-3.66 (7H, m), 3.76-3.79 (2H, m), 3.94-3.99 (3H, m), 4.07-4.14 (1H, m), 5.78 (1H, s).

### Example 24. Synthesis of N-methyl-N-((4-(methylsulfonyl)thiomorpholin-2-yl)methyl)-6-(1-oxa-4-azaspiro[5.5]undecan-4-yl)-2-(trifluoromethyl)pyrimidin-4-amine (Compound 26)

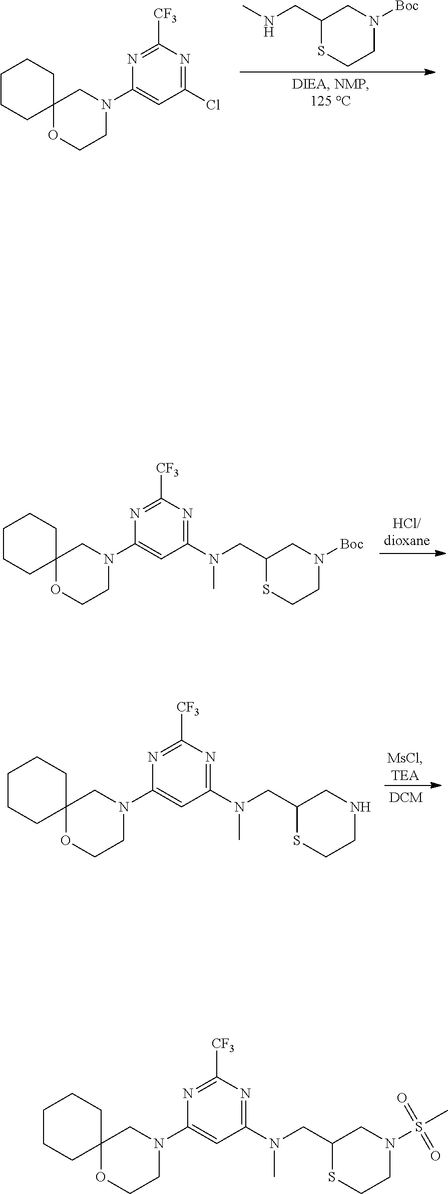 [SMILES: FC(F)(F)c1nc(Cl)cc(N2CCOC3(CCCCC3)C2)n1 ; CNCC1CN(C(=O)OC(C)(C)C)CCS1 ; CN(CC1CN(C(=O)OC(C)(C)C)CCS1)c1cc(N2CCOC3(CCCCC3)C2)nc(C(F)(F)F)n1 ; CN(CC1CNCCS1)c1cc(N2CCOC3(CCCCC3)C2)nc(C(F)(F)F)n1 ; "MsCl,TEA" ; CN(CC1CN(S(C)(=O)=O)CCS1)c1cc(N2CCOC3(CCCCC3)C2)nc(C(F)(F)F)n1]

### Step A. Synthesis of tert-butyl 2-(((6-(1-oxa-4-azaspiro[5.5]undecan-4-yl)-2-(trifluoromethyl)pyrimidin-4-yl)(methyl)amino)methyl)thiomorpholine-4-carboxylate

To a solution of 4-(6-chloro-2-(trifluoromethyl)pyrimidin-4-yl)-1-oxa-4-azaspiro[5.5]undecane (65 mg, 0.19 mmol, 1.00 equiv) and tert-butyl 2-((methylamino)methyl)thiomorpholine-4-carboxylate (HCl salt) (81 mg, 0.29 mmol, 1.50 equiv) in NMP (2 mL) was added DIEA (74 mg, 0.57 mmol, 3.00 equiv). The mixture was stirred at 125 °C for 16 h. The mixture was extracted with EtOAc and washed with brine. The organic layer was dried over Na₂SO₄ and concentrated. The residue was purified by column eluted with PE/EA=4/1 to get tert-butyl 2-(((6-(1-oxa-4-azaspiro[5.5]undecan-4-yl)-2-(trifluoromethyl)pyrimidin-4-yl)(methyl)amino)methyl)thiomorpholine-4-carboxylate (65 mg, 61.3%) as colorless oil. LCMS: [M+H]⁺=546.

### Step B. Synthesis of N-methyl-6-(1-oxa-4-azaspiro[5.5]undecan-4-yl)-N-(thiomorpholin-2-ylmethyl)-2-(trifluoromethyl)pyrimidin-4-amine (HCl Salt)

To a solution of tert-butyl 2-(((6-(1-oxa-4-azaspiro[5.5]undecan-4-yl)-2-(trifluoromethyl)pyrimidin-4-yl)(methyl)amino)methyl)thiomorpholine-4-carboxylate (65 mg, 0.12 mmol, 1.00 equiv) in DCM (3 mL) was added 1 mL HCl (4N in 1,4-dioxane. The mixture was stirred at room temperature for 1 h. The mixture was concentrated to get the crude N-methyl-6-(1-oxa-4-azaspiro[5.5]undecan-4-yl)-N-(thiomorpholin-2-ylmethyl)-2-(trifluoromethyl)pyrimidin-4-amine (HCl salt) (62 mg, >100%) as colorless oil. LCMS: [M+H]⁺=446.

### Step C. Synthesis of N-methyl-N-((4-(methylsulfonyl)thiomorpholin-2-yl)methyl)-6-(1-oxa-4-azaspiro[5.5]undecan-4-yl)-2-(trifluoromethyl)pyrimidin-4-amine

To a solution of N-methyl-6-(1-oxa-4-azaspiro[5.5]undecan-4-yl)-N-(thiomorpholin-2-ylmethyl)-2-(trifluoromethyl)pyrimidin-4-amine (HCl salt) (62 mg, 0.13 mmol, 1.00 equiv) and TEA (39 mg, 0.39 mmol, 3.00 equiv) in DCM (4 mL) was added MsCl (22 mg, 0.19 mmol, 1.50 equiv). The mixture was stirred at room temperature for 1 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 50% B to 95% B in 10 min, Wavelength: 214 nm. This afforded the title compound (35 mg, 52%) as white solid. LCMS: [M+H]⁺=524.2. ¹H NMR (400 MHz, CDCl₃): δ 1.35-1.61 (8H, m), 1.72-1.75 (2H, m), 2.73-2.76 (4H, m), 2.84-2.88 (1H, m), 3.06 (3H, s), 3.27-3.41 (3H, m), 3.50 (2H, s), 3.58-3.73 (5H, m), 3.77-3.79 (2H, m), 3.90-3.95 (1H, m), 5.40 (1H, s).

### Example 25. Synthesis of 4-(6-(methyl((4-(methylsulfonyl)morpholin-2-yl)methyl)amino)-2-(trifluoromethyl)pyrimidin-4-yl)-1-thia-4-azaspiro[5.5]undecane 1,1-dioxide (Compound 27)

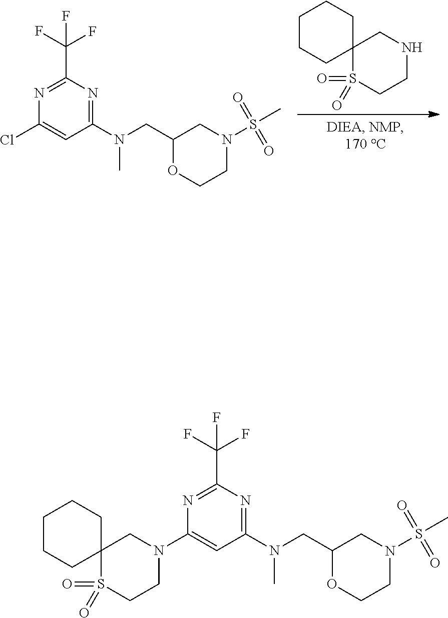 [SMILES: CN(CC1CN(S(C)(=O)=O)CCO1)c1cc(Cl)nc(C(F)(F)F)n1 ; O=S1(=O)CCNCC12CCCCC2 ; CN(CC1CN(S(C)(=O)=O)CCO1)c1cc(N2CCS(=O)(=O)C3(CCCCC3)C2)nc(C(F)(F)F)n1]

To a solution of 6-chloro-N-methyl-N-((4-(methylsulfonyl)morpholin-2-yl)methyl)-2-(trifluoromethyl)pyrimidin-4-amine (70 mg, 0.18 mmol, 1.00 equiv) and 1-thia-4-azaspiro[5.5]undecane 1,1-dioxide (44 mg, 0.22 mmol, 1.20 equiv) in NMP (4 mL) was added DIEA (70 mg, 0.54 mmol, 3.00 equiv). The mixture was stirred at 170 °C for 3 h with microwave. The mixture was stirred at 170 °C for 6 h with microwave. The mixture was extracted with EtOAc and washed with brine. The organic layer was dried over Na₂SO₄ and concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 45% B to 75% B in 12 min. Wavelength: 214 nm. This afforded the title compound (25 mg, 25%) as white solid. LCMS: [M+H]⁺=556.1. ¹H NMR (400 MHz, CDCl₃): δ 1.25-1.32 (1H, m), 1.61-1.64 (2H, m), 1.74-1.77 (5H, m), 1.88-1.96 (2H, m), 2.60-2.66 (1H, m), 2.76 (3H, s), 2.79-2.86 (1H, m), 3.04-3.08 (5H, m), 3.43-3.81 (5H, m), 3.98-4.05 (4H, m), 4.17-4.29 (2H, m), 5.53 (1H, s).

### Example 26. Synthesis of N-methyl-N-((4-(methylsulfonyl)morpholin-2-yl)methyl)-6-(1-thia-4-azaspiro[5.5]undecan-4-yl)-2-(trifluoromethyl)pyrimidin-4-amine (Compound 28)

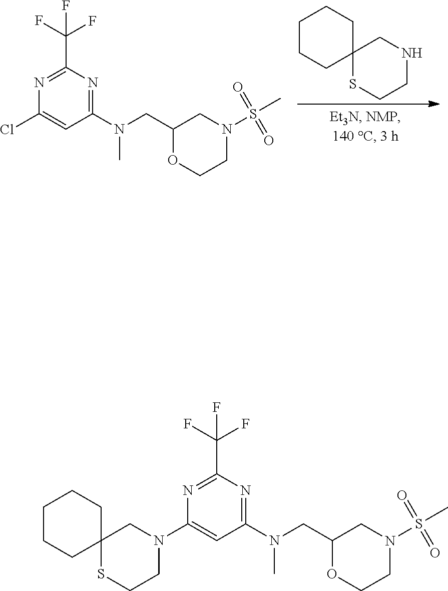 [SMILES: CN(CC1CN(S(C)(=O)=O)CCO1)c1cc(Cl)nc(C(F)(F)F)n1 ; C1CCC2(CC1)CNCCS2 ; CN(CC1CN(S(C)(=O)=O)CCO1)c1cc(N2CCSC3(CCCCC3)C2)nc(C(F)(F)F)n1]

To a solution of 6-chloro-N-methyl-N-((4-(methylsulfonyl)morpholin-2-yl)methyl)-2-(trifluoromethyl)pyrimidin-4-amine (136 mg, 0.351 mmol, 1.20 equiv) in NMP (2 mL) was added -thia-4-azaspiro[5.5]undecane (50 mg, 0.292 mmol, 1.00 equiv) and Et₃N (0.2 mL, 1.459 mmol, 5.00 equiv) and then heated to 140 °C. The reaction was stirred at 140 °C for 3 h. The reaction was diluted with EA and washed with water and brine. The organic layer was dried over anhydrous sodium sulfate, filtered and concentrated to obtain crude product. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 65% B to 95% B in 10 min, Wavelength: 214 nm. This afforded the title compound (50 mg, 33%) as yellow solid. LCMS: [M+H]⁺=524.1. ¹H NMR (400 MHz, DMSO-d₆): δ1.24-1.60 (11H, m), 2.56-2.67 (3H, m), 2.80-2.86 (4H, m), 3.06 (3H, s), 3.39-3.62 (3H, m), 3.69-3.73 (2H, m), 3.87-3.95 (5H, m), 5.84 (1H, s).

### Example 27. Synthesis of 4-(6-(methyl((4-(methylsulfonyl)-1,1-dioxidothiomorpholin-2-yl)methyl)amino)-2-(trifluoromethyl)pyrimidin-4-yl)-1-thia-4-azaspiro[5.5]undecane 1,1-dioxide (Compound 29)

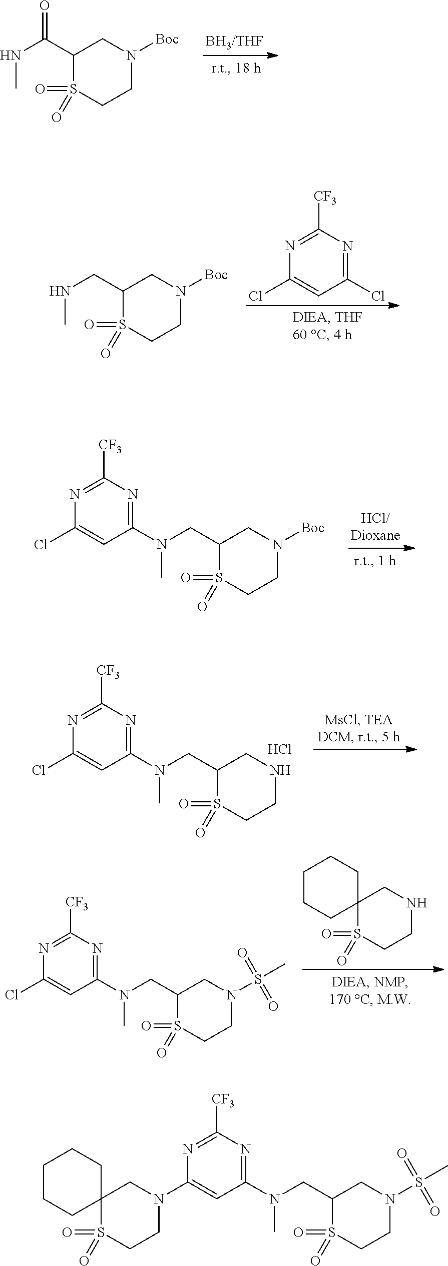

### Step A. Synthesis of tert-butyl 2-((methylamino)methyl)thiomorpholine-4-carboxylate 1,1-dioxide

The mixture of tert-butyl 2-(methylcarbamoyl)thiomorpholine-4-carboxylate 1,1-dioxide (50 mg, 0.17 mmol, 1.00 equiv.) in BH₃/THF (1M, 5 mL) was stirred at r.t. for 18 h. The mixture was quenched with MeOH and concentrated in vacuum to afford tert-butyl 2-((methylamino)methyl)thiomorpholine-4-carboxylate 1,1-dioxide (150 mg, >100%) as colorless oil. LCMS: [M+H]⁺=279.2.

### Step B. Synthesis of tert-butyl 2-(((6-chloro-2-(trifluoromethyl)pyrimidin-4-yl)(methyl)amino)methyl)thiomorpholine-4-carboxylate 1,1-dioxide

To a solution of 4,6-dichloro-2-(trifluoromethyl)pyrimidine (37 mg, 0.17 mmol, 1.00 equiv.) in THF (5 mL) was added DIEA (44 mg, 0.34 mmol, 2.00 equiv.) and tert-butyl 2-((methylamino)methyl)thiomorpholine-4-carboxylate 1,1-dioxide (150 mg, 0.17 mmol, 1.00 equiv.) at r.t. The mixture was stirred at 60 °C for 4 h. The mixture was extracted with EA (10.00 mL×3) and water (10.00 mL). The organic layer was dried over Na₂SO₄ and concentrated in vacuum to remove the solvent. The residue was purified by flash chromatography (EA/PE=60%) to afford tert-butyl 2-(((6-chloro-2-(trifluoromethyl)pyrimidin-4-yl)(methyl)amino)methyl)thiomorpholine-4-carboxylate 1,1-dioxide (80 mg, >100%) as yellow oil. LCMS: [M+H]⁺=459.0.

### Step C. Synthesis of 2-(((6-chloro-2-(trifluoromethyl)pyrimidin-4-yl)(methyl)amino)methyl)thiomorpholine 1,1-dioxide

A mixture of tert-butyl 2-(((6-chloro-2-(trifluoromethyl)pyrimidin-4-yl)(methyl)amino)methyl)thiomorpholine-4-carboxylate 1,1-dioxide (80 mg, 0.17 mmol, 1.00 equiv.) in HCl/Dioxane (4M, 5 mL) was stirred at r.t. for 1 h. The mixture was concentrated in vacuum to remove the solvent to afford 2-(((6-chloro-2-(trifluoromethyl)pyrimidin-4-yl)(methyl)amino)methyl)thiomorpholine 1,1-dioxide (100 mg, >100%) as white solid. LCMS: [M+H]⁺=359.1.

### Step D. Synthesis of 2-(((6-chloro-2-(trifluoromethyl)pyrimidin-4-yl)(methyl)amino)methyl)-4-(methylsulfonyl)thiomorpholine 1,1-dioxide

To a solution of 2-(((6-chloro-2-(trifluoromethyl)pyrimidin-4-yl)(methyl)amino)methyl)thiomorpholine 1,1-dioxide (100 mg, 0.17 mmol, 1.00 equiv.) in DCM (5 mL) was added DIEA (44 mg, 0.34 mmol, 2.00 equiv.) and MsCl (58 mg, 0.51 mmol, 3.00 equiv) at r.t. The mixture was stirred at r.t. for 5 h. The mixture was concentrated in vacuum to remove the solvent. The residue was purified by flash chromatography (EA/PE=50%) to afford 2-(((6-chloro-2-(trifluoromethyl)pyrimidin-4-yl)(methyl)amino)methyl)-4-(methylsulfonyl)thiomorpholine 1,1-dioxide (67 mg, 90%) as white solid. LCMS: [M+H]⁺=437.0.

### Step E. Synthesis of 4-(6-(methyl((4-(methylsulfonyl)-1,1-dioxidothiomorpholin-2-yl)methyl)amino)-2-(trifluoromethyl)pyrimidin-4-yl)-1-thia-4-azaspiro[5.5]undecane 1,1-dioxide

To a solution of 2-(((6-chloro-2-(trifluoromethyl)pyrimidin-4-yl)(methyl)amino)methyl)thiomorpholine 1,1-dioxide (67 mg, 0.15 mmol, 1.00 equiv.) in NMP (5 mL) was added DIEA (40 mg, 0.31 mmol, 2.00 equiv) and 1-thia-4-azaspiro[5.5]undecane 1,1-dioxide (62 mg, 0.31 mmol, 2.00 equiv) at r.t. The mixture was stirred at 170 °C in a microwave for 5 h. The mixture was extracted with EA (10.00 mL×3) and water (10.00 mL). The organic layer was dried over Na₂SO₄ and concentrated in vacuum to remove the solvent. The residue was purified by Prep-HPLC with the following conditions: Column: XBridge C18 SN271/3611211212 Waters, 19×150 mm, 5 m; Mobile Phase A: Water (0.1% NH₄HCO₃), Mobile Phase B: ACN; Flow rate: 15 mL/min; Gradient: 30% B to 80% B in 9 min. Wavelength: 214 nm. This afforded the title compound (18 mg, yield 20%) as white solid. LCMS: [M+H]⁺=604.1. H NMR (400 MHz, CDCl₃): δ 1.16-1.38 (1H, m), 1.60-1.68 (2H, m), 1.68-1.82 (5H, m), 1.84-1.99 (2H, m), 2.90 (3H, s), 3.04-3.10 (2H, m), 3.13 (3H, s), 3.14-3.27 (2H, m), 3.41-3.52 (2H, m), 3.55-3.66 (1H, m), 3.76-3.86 (1H, m), 4.00-4.30 (7H, m), 5.62 (1H, s)

### Example 28. Synthesis of N-methyl-N-((4-(methylsulfonyl)thiomorpholin-2-yl)methyl)-6-(1-thia-4-azaspiro[5.5]undecan-4-yl)-2-(trifluoromethyl)pyrimidin-4-amine (Compound 30)

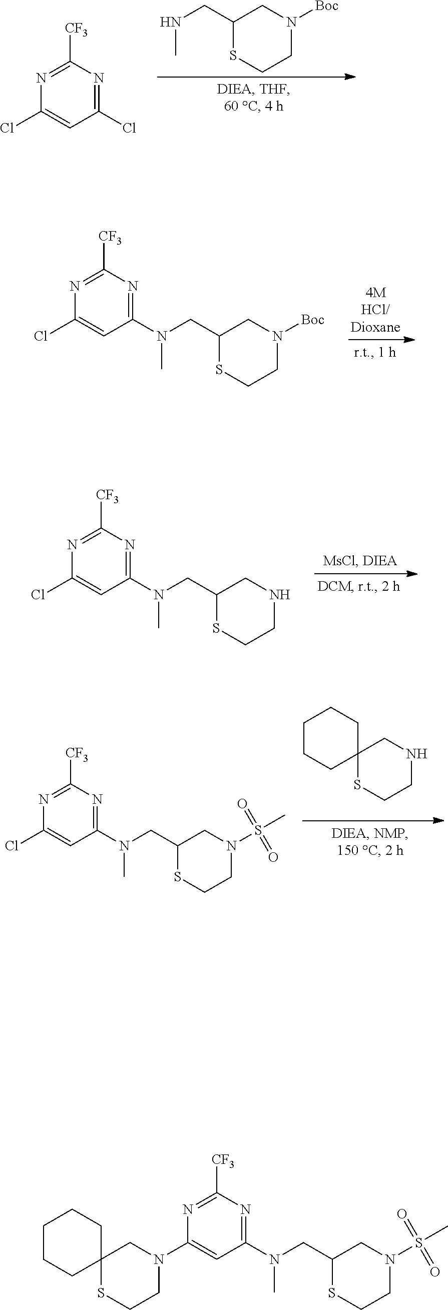

### Step A. Synthesis of tert-butyl 2-(((6-chloro-2-(trifluoromethyl)pyrimidin-4-yl)(methyl)amino)methyl)thiomorpholine-4-carboxylate

To a solution of 4,6-dichloro-2-(trifluoromethyl)pyrimidine (100 mg, 0.46 mmol, 1.00 equiv.) in THF (10 mL) was added DIEA (119 mg, 0.92 mmol, 2.00 equiv.) and tert-butyl 2-((methylamino)methyl)thiomorpholine-4-carboxylate (113 mg, 0.46 mmol, 1.00 equiv.) at r.t. The mixture was stirred at 60 °C for 4 h. The mixture was extracted with EA (10.00 mL×3) and water (10.00 mL). The organic layer was dried over Na₂SO₄ and concentrated in vacuum to remove the solvent. The residue was purified by flash chromatography (EA/PE=20%) to afford tert-butyl 2-(((6-chloro-2-(trifluoromethyl)pyrimidin-4-yl)(methyl)amino)methyl)thiomorpholine-4-carboxylate (114 mg, 58%) as colorless oil. LCMS: [M+H]⁺=427.1.

### Step B. Synthesis of 6-chloro-N-methyl-N-(thiomorpholin-2-ylmethyl)-2-(trifluoromethyl)pyrimidin-4-amine

A mixture of tert-butyl 2-(((6-chloro-2-(trifluoromethyl)pyrimidin-4-yl)(methyl)amino)methyl)thiomorpholine-4-carboxylate (114 mg, 0.27 mmol, 1.00 equiv.) in HCl/Dioxane (4M, 10 mL) was stirred at r.t. for 1 h. The mixture was concentrated in vacuum to remove the solvent to afford 6-chloro-N-methyl-N-(thiomorpholin-2-ylmethyl)-2-(trifluoromethyl)pyrimidin-4-amine (160 mg, >100%) as white solid. LCMS: [M+H]⁺=327.0.

### Step C. Synthesis of 6-chloro-N-methyl-N-((4-(methylsulfonyl)thiomorpholin-2-yl)methyl)-2-(trifluoromethyl)pyrimidin-4-amine

To a solution of 6-chloro-N-methyl-N-(thiomorpholin-2-ylmethyl)-2-(trifluoromethyl)pyrimidin-4-amine (160 mg, 0.27 mmol, 1.00 equiv.) in DCM (5 mL) was added DIEA (70 mg, 0.54 mmol, 2.00 equiv.) and MsCl (62 mg, 0.54 mmol, 2.00 equiv) at r.t. The mixture was stirred at r.t. for 2 h. The mixture was concentrated in vacuum to remove the solvent. The residue was purified by flash chromatography (EA/PE=50%) to afford 6-chloro-N-methyl-N-((4-(methylsulfonyl)thiomorpholin-2-yl)methyl)-2-(trifluoromethyl)pyrimidin-4-amine (73 mg, 67%) as yellow oil. LCMS: [M+H]⁺=405.

### Step D. Synthesis of N-methyl-N-((4-(methylsulfonyl)thiomorpholin-2-yl)methyl)-6-(1-thia-4-azaspiro[5.5]undecan-4-yl)-2-(trifluoromethyl)pyrimidin-4-amine

To a solution of 6-chloro-N-methyl-N-((4-(methylsulfonyl)thiomorpholin-2-yl)methyl)-2-(trifluoromethyl)pyrimidin-4-amine (73 mg, 0.18 mmol, 1.00 equiv) in NMP (5 mL) was added DIEA (47 mg, 0.36 mmol, 2.00 equiv) and 1-thia-4-azaspiro[5.5]undecane (42 mg, 0.36 mmol, 2.00 equiv) at r.t. The mixture was stirred at 150 °C for 2 h. The mixture was extracted with EA (10.00 mL×3) and water (10.00 mL). The organic layer was dried over Na₂SO₄ and concentrated in vacuum to remove the solvent. The residue was purified by Prep-HPLC with the following conditions: Column: XBridge C18 SN271/3611211208 Waters, 19×150 mm, 5 μm; Mobile Phase A: Water (0.1% NH₄HCO₃), Mobile Phase B: ACN; Flow rate: 15 mL/min; Gradient: 70% B to 90% B in 8 min. Wavelength: 214 nm. This afforded the title compound (28 mg, yield 29%) as off-white solid. LCMS: [M+H]⁺=540.1. ¹H NMR (400 MHz, CDCl₃): δ 1.22-1.56 (6H, m), 1.59-1.67 (2H, m), 1.67-1.77 (2H, m), 2.64-2.79 (6H, m), 2.82-2.92 (1H, m), 3.06 (3H, s), 3.24-3.35 (2H, m), 3.35-3.44 (1H, m), 3.55-3.65 (2H, m), 3.66-3.75 (1H, m), 3.82-3.97 (5H, m), 5.41 (1H, s)

### Example 29. Synthesis of N-methyl-N-((4-(methylsulfonyl)-1,4-oxazepan-2-yl)methyl)-6-(1-oxa-4-azaspiro[5.5]undecan-4-yl)-2-(trifluoromethyl)pyrimidin-4-amine (Compound 31)

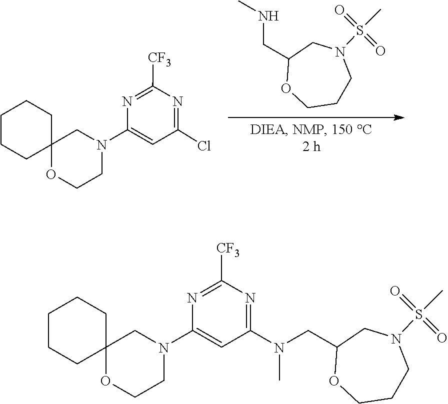

To a solution of 4-(6-chloro-2-(trifluoromethyl)pyrimidin-4-yl)-1-oxa-4-azaspiro[5.5]undecane (100 mg, 0.30 mmol, 1.00 equiv) in NMP (5 mL) was added DIEA (77 mg, 0.60 mmol, 2.00 equiv) and N-methyl-1-(4-(methylsulfonyl)-1,4-oxazepan-2-yl)methanamine (66 mg, 0.30 mmol, 1.00 equiv) at r.t. The mixture was stirred at 140 °C for 2 h. The mixture was extracted with EA (10.00 mL×3) and water (10.00 mL). The organic layer was dried over Na₂SO₄ and concentrated in vacuum to remove the solvent. The residue was purified by Prep-HPLC with the following conditions: Column: XBridge C18 SN271/3611211208 Waters, 19×150 mm, 5 μm; Mobile Phase A: Water (0.1% NH₄HCO₃), Mobile Phase B: ACN; Flow rate: 15 mL/min; Gradient: 60% B to 85% B in 10 min. Wavelength: 214 nm. This afforded the title compound (68 mg, 43%) as a white solid. LCMS: [M+H]⁺=522.2. ¹H NMR (400 MHz, CDCl₃): δ 1.19-1.67 (8H, m), 1.68-1.82 (2H, m), 1.90-2.05 (2H, m), 2.82 (3H, s), 2.92-3.02 (1H, m), 3.05 (3H, s), 3.18-3.30 (1H, m), 3.44-3.56 (3H, m), 3.56-3.70 (4H, m), 3.70-3.82 (3H, m), 3.82-3.93 (2H, m), 4.04-4.09 (1H, m), 5.40 (1H, m).

### Example 30. Synthesis of N-((4-(cyclopropylsulfonyl)morpholin-2-yl)methyl)-N-methyl-6-(1-oxa-4-azaspiro[5.5]undecan-4-yl)-2-(trifluoromethyl)pyrimidin-4-amine (Compound 32)

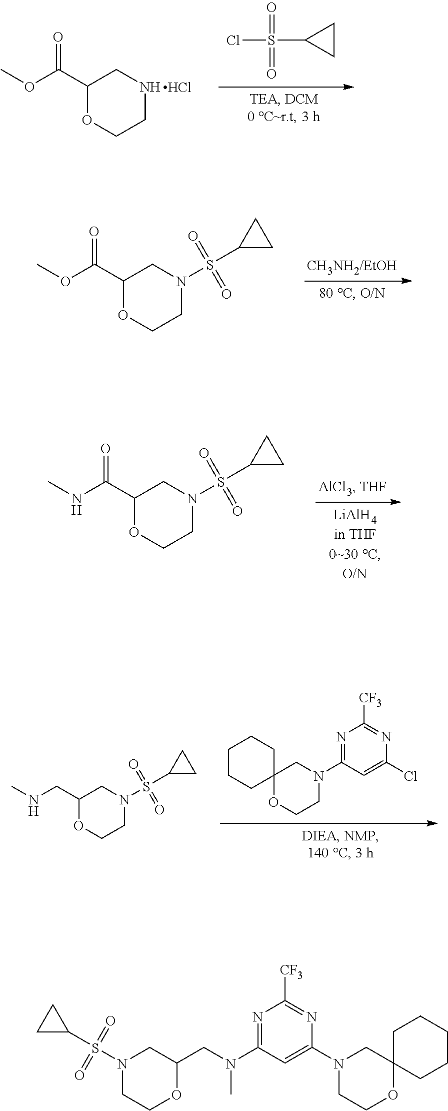

### Step A. Synthesis of methyl 4-(cyclopropylsulfonyl)morpholine-2-carboxylate

To a solution of methyl morpholine-2-carboxylate hydrochloride (200 mg, 1.10 mmol, 1.00 equiv) in dry DCM (10 mL) at 0 °C was added TEA (335 mg, 3.30 mmol, 3.00 equiv) and stirred at r.t for 15 minutes, then added cyclopropanesulfonyl chloride (231 mg, 1.65 mmol, 1.50 equiv). The mixture was stirred at r.t for 3 h. The mixture was extracted with DCM, washed with water and brine, dried over Na₂SO₄, filtered and concentrated to get methyl 4-(cyclopropylsulfonyl)morpholine-2-carboxylate (300 mg, >100% purity) as yellow oil, which was used to the next step without purification. LCMS: 250 [M+H]⁺

### Step B. Synthesis of 4-(cyclopropylsulfonyl)-N-methylmorpholine-2-carboxamide

To a solution of methyl 4-(cyclopropylsulfonyl)morpholine-2-carboxylate (300 mg, 1.20 mmol, 1.00 equiv) in 33% CH₃NH₂ in EtOH (15 mL) was heated to 80 °C and stirred at 80 °C for overnight. The reaction was concentrated to get crude solid, washed with PE to get 4-(cyclopropylsulfonyl)-N-methylmorpholine-2-carboxamide (300 mg, >100%) as yellow solid, which was used to the next step without purification. LCMS: m/z=249 [M+H]⁺

### Step C. Synthesis of 1-(4-(cyclopropylsulfonyl)morpholin-2-yl)-N-methyl-methanamine

To a solution of AlCl₃ (162 mg, 1.21 mmol, 1.00 equiv) in dry THF (10 mL) was cooled to 0 °C and added 1M lithium aluminum hydride in THF (3.6 ml, 3.63 mmol, 3.00 equiv) dropwise over 3 minutes. The reaction was stirred at 0 °C for 0.5 h and added dropwise a solution of 4-(cyclopropylsulfonyl)-N-methylmorpholine-2-carboxamide (300 mg, 1.21 mmol, 1.00 equiv) in dry THF (10 mL) at 0 °C. The reaction was stirred at r.t for overnight. Water, 15% sodium hydroxide and additional water were added. The reaction was stirred an additional 30 minutes. The reaction was filtered and washed with MeOH. The filtrates were combined and concentrated to get 1-(4-(cyclopropylsulfonyl)morpholin-2-yl)-N-methyl-methanamine (300 mg, >100%) as yellow oil, which was used to the next step without purification. LCMS: [M+H]⁺=235.

### Step D. Synthesis of N-((4-(cyclopropylsulfonyl)morpholin-2-yl)methyl)-N-methyl-6-(1-oxa-4-azaspiro[5.5]undecan-4-yl)-2-(trifluoromethyl)pyrimidin-4-amine

To a solution of 4-(6-chloro-2-(trifluoromethyl)pyrimidin-4-yl)-1-oxa-4-azaspiro[5.5]undecane (100 mg, 0.30 mmol, 1.00 equiv) in NMP (10 mL) was added 1-(4-(cyclopropylsulfonyl)morpholin-2-yl)-N-methylmethanamine (280 mg, 1.20 mmol, 4.00 equiv) and DIEA (77 mg, 0.60 mmol, 2.0 equiv) and heated to 140 °C. The reaction was stirred at 140 °C for 3 h. The reaction was diluted with EA and washed with water and brine. The organic layer was dried over anhydrous sodium sulfate, filtered, and concentrated to obtain crude product. The residue was purified by using Prep-HPLC with the following conditions: Column: Sunfire C18 OBD Column, 19×150 mm, 5 μm; Mobile Phase A: Water (0.1% TFA), Mobile Phase B: ACN; Flow rate: 15 mL/min; Gradient: 67% B to 72% B in 12 min, Wavelength: 214 nm. This afforded the title compound (16 mg, 10%) as white solid. LCMS-MS m/z=534.2 [M+H]⁺. ¹H NMR (400 MHz, CDCl₃): δ 0.95-1.02 (2H, m), 1.09-1.18 (2H, m), 1.33-1.50 (6H, m), 1.58-1.64 (2H, m), 1.72-1.75 (2H, m), 2.19-2.25 (1H, m), 2.75-2.81 (1H, m), 2.95-3.01 (1H, m), 3.04 (3H, s), 3.41-3.68 (8H, m), 3.74-3.80 (3H, m), 3.98 (1H, m), 4.08 (1H, m), 5.40 (1H, s).

### Example 31. Synthesis of N-((4-(ethylsulfonyl) morpholin-2-yl)methyl)-N-methyl-6-(1-oxa-4-azaspiro[5.5]undecan-4-yl)-2-(trifluoromethyl)pyrimidin-4-amine (Compound 33)

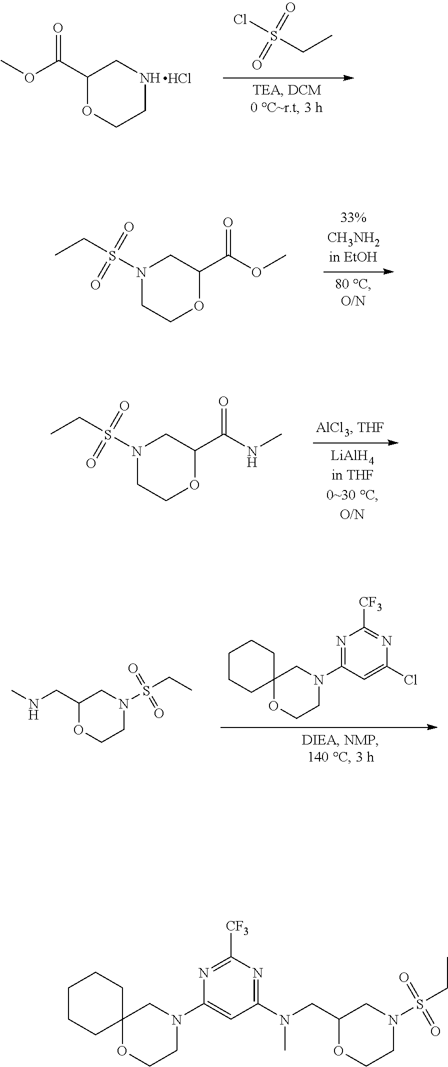

### Step A. Synthesis of methyl 4-(ethylsulfonyl) morpholine-2-carboxylate

To a solution of methyl morpholine-2-carboxylate hydrochloride (200 mg, 1.10 mmol, 1.00 equiv) in dry DCM (10 mL) at 0 °C was added TEA (335 mg, 3.30 mmol, 3.00 equiv) and ethylsulfonyl chloride (211 mg, 1.65 mmol, 1.50 equiv). The mixture was stirred at r.t for 3 h. The reaction was checked by LC-MS and the reaction was finished. The mixture was added water and extracted with DCM, washed with water and brine. The combined organic layer was dried over Na₂SO₄, filtered and concentrated to get methyl 4-(ethylsulfonyl) morpholine-2-carboxylate (300 mg, >100%) as yellow oil, which was used to the next step without purification. LCMS: [M+H]⁺=238.

### Step B. Synthesis of 4-(ethylsulfonyl)-N-methylmorpholine-2-carboxamide

To a solution of methyl 4-(ethylsulfonyl) morpholine-2-carboxylate (300 mg, 1.27 mmol, 1.00 equiv) in 33% CH₃NH₂ in EtOH (15 mL) was heated to 80 °C and stirred at 80 °C for overnight. The reaction was concentrated to get crude solid, washed with PE to get 4-(ethylsulfonyl)-N-methylmorpholine-2-carboxamide (270 mg, 90%) as yellow solid, which was used to the next step without purification. LCMS: 237 [M+H]⁺

### Step C. Synthesis of 1-(4-(ethylsulfonyl) morpholin-2-yl)-N-methylmethanamine

To a solution of AlCl₃ (153 mg, 1.14 mmol, 1.00 equiv) in dry THF (10 mL) was cooled to 0° Cand added 1M lithium aluminum hydride in THF (3.4 ml, 3.42 mmol, 3.00 equiv) dropwise over 3 minutes. The reaction was stirred at 0 °C for 0.5 h and then added dropwise a solution of 4-(ethylsulfonyl)-N-methylmorpholine-2-carboxamide (270 mg, 1.14 mmol, 1.0 equiv) in dry THF (10 mL) slowly at 0 °C. The reaction was stirred at r.t for overnight. Water, 15% sodium hydroxide and additional water were added, and the reaction was stirred an additional 30 minutes. The reaction was filtered, and the solids were washed with MeOH. The filtrates were combined and concentrated to get 1-(4-(ethylsulfonyl) morpholin-2-yl)-N-methylmethanamine (300 mg, >100%) as yellow oil, which was used to the next step without purification. LCMS: 223 [M+H]⁺

### Step D. Synthesis of N-((4-(ethylsulfonyl) morpholin-2-yl)methyl)-N-methyl-6-(1-oxa-4-azaspiro[5.5]undecan-4-yl)-2-(trifluoromethyl)pyrimidin-4-amine

To a solution of 4-(6-chloro-2-(trifluoromethyl)pyrimidin-4-yl)-1-oxa-4-azaspiro[5.5]undecane (100 mg, 0.30 mmol, 1.00 equiv) in NMP (10 mL) was added 1-(4-(ethylsulfonyl)morpholin-2-yl)-N-methylmethanamine (267 mg, 1.20 mmol, 4.00 equiv) and DIEA (77 mg, 0.60 mmol, 2.00 equiv) at R.T. The reaction was stirred at 140 °C for 3 h. The reaction was diluted with EA and washed with water and brine. The organic layer was dried over anhydrous sodium sulfate, filtered, and concentrated to obtain crude product. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 55% B to 95% B in 10 min, Wavelength: 214 nm. This afforded the title compound (42 mg, 27%) as white solid. LCMS-MS m/z=522.2 [M+H]⁺. ¹H NMR (400 MHz, CDCl₃): δ 1.35 (3H, J=7.4 Hz, t), 1.41-1.52 (6H, m), 1.58-1.64 (2H, m), 1.72-176 (2H, m), 2.72-2.77 (1H, m), 2.90-2.98 (3H, m), 3.04 (3H, s), 3.41-3.65 (8H, m), 3.71-3.79 (3H, m), 3.95-4.05 (2H, m), 5.40 (1H, s).

### Example 32. Synthesis of 4-(6-(1-(methylsulfonyl)octahydro-6H-pyrido[3,4-b][1,4]oxazin-6-yl)-2-(trifluoromethyl)pyrimidin-4-yl)-1-oxa-4-azaspiro[5.5]undecane (Compound 34)

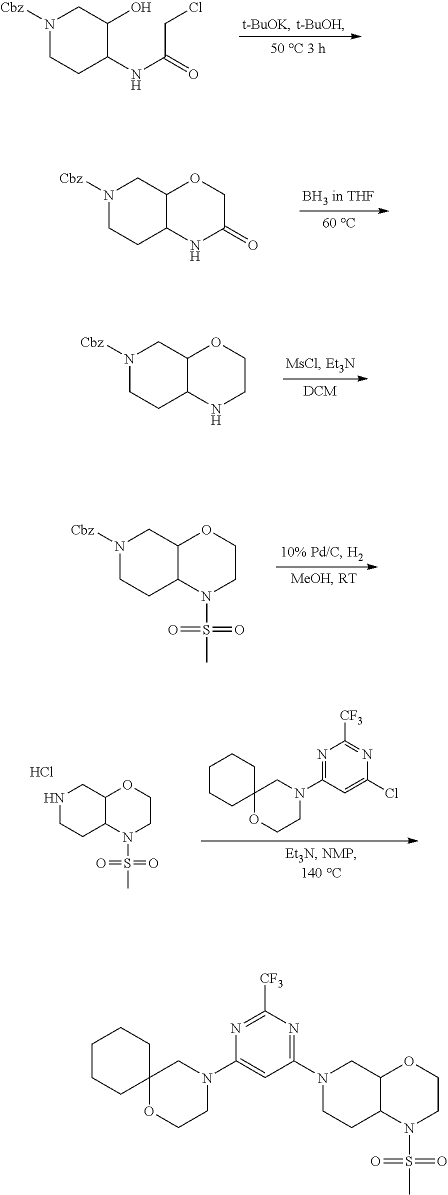

### Step A. Synthesis of benzyl 2-oxooctahydro-6H-pyrido[3,4-b][1,4]oxazine-6-carboxylate

To a solution of t-BuOK (1.83 g, 16.38 mmol 1.50 equiv) in t-BuOH (150 mL) was added benzyl 4-(2-chloroacetamido)-3-hydroxypiperidine-1-carboxylate (3.56 g, 10.92 mmol, 1.00 equiv) dropwise at 50 °C. The mixture was stirred at 50 °C for 2 h. The mixture was concentrated in vacuo and the residue purifying by flash chromatography (MeOH/DCM=3%) to afford benzyl 2-oxohexahydro-1H-pyrido[3,4-b][1,4]oxazine-6(7H)-carboxylate (1.49 g, 47%) as white solid. LCMS: [M+H]⁺=291.

### Step B. Synthesis of benzyl octahydro-6H-pyrido[3,4-b][1,4]oxazine-6-carboxylate

To a solution of benzyl 2-oxohexahydro-1H-pyrido[3,4-b][1,4]oxazine-6(7H)-carboxylate (1.00 g, 3.45 mmol, 1.00 equiv) in BH₃ in THF (50 mL, 1M) at 0 °C. The reaction was stirred at 60 °C for 3 h. The reaction was evaporated under reduce pressure to get benzyl octahydro-6H-pyrido[3,4-b][1,4]oxazine-6-carboxylate crude (700 mg, 73%) as colorless oil, which was used to the next step without purification. LCMS: [M+H]⁺=277.

### Step C. Synthesis of benzyl 1-(methylsulfonyl)octahydro-6H-pyrido[3,4-b][1,4]oxazine-6-carboxylate

To a solution of benzyl octahydro-6H-pyrido[3,4-b][1,4]oxazine-6-carboxylate (650 mg, 2.35 mmol, 1.00 equiv) in DCM (10 mL) was added Et₃N (1.63 mL, 11.78 mmol, 5.00 equiv) and MsCl (539 mg, 4.71 mmol, 2.00 equiv) at 0 °C. The reaction was stirred at 25 °C for 4 h. The reaction was diluted with DCM and washed with water and brine. The organic layer was dried over anhydrous sodium sulfate, filtered and concentrated to obtain crude product. The crude product was purified by flash chromatography (EA/PE=10%) to give benzyl 1-(methylsulfonyl)octahydro-6H-pyrido[3,4-b][1,4]oxazine-6-carboxylate (523 mg, 58%) as yellow oil. LCMS: [M+H]⁺=355.

### Step D. Synthesis of 1-(methylsulfonyl)octahydro-1H-pyrido[3,4-b][1,4]oxazine

To a solution of benzyl 1-(methylsulfonyl)octahydro-6H-pyrido[3,4-b][1,4]oxazine-6-carboxylate (523 mg, 1.48 mmol, 1.00 equiv) in MeOH (5 mL) was added 10% Pd/C (80 mg). The reaction was stirred at 25 °C for 16 h under H₂. The reaction was filtered and the filtrate was adjusted to pH=1-2 with conc. HCl. The mixture was evaporated aqueous to obtain crude product 1-(methylsulfonyl)octahydro-1H-pyrido[3,4-b][1,4]oxazine (362 mg, >100%) as white solid. LCMS: [M+H]⁺=220.

### Step E. Synthesis of 4-(6-(1-(methylsulfonyl)octahydro-6H-pyrido[3,4-b][1,4]oxazin-6-yl)-2-(trifluoromethyl)pyrimidin-4-yl)-1-oxa-4-azaspiro[5.5]undecane

To a solution of 1-(methylsulfonyl)octahydro-1H-pyrido[3,4-b][1,4]oxazine (110 mg, 0.50 mmol, 1.00 equiv) in NMP (3 mL) was added 4-(6-chloro-2-(trifluoromethyl)pyrimidin-4-yl)-1-oxa-4-azaspiro[5.5]undecane (151 mg, 0.45 mmol, 0.90 equiv) and Et₃N (253 mg, 2.50 mmol, 5.00 equiv). The reaction was stirred at 140 °C for 4 h. The reaction was diluted with EA and washed with water and brine. The organic layer was dried over anhydrous sodium sulfate, filtered and concentrated to obtain crude product. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 62% B to 73% B in 12 min, Wavelength: 214 nm to afford the title compound (57 mg, 32%) as white solid. LCMS: [M+H]⁺=520.2. ¹H NMR (400 MHz, DMSO-d₆): δ 1.24-1.63 (12H, m), 2.32-2.43 (1H, m), 2.66-2.75 (1H, m), 2.83-2.92 (1H, m), 2.94-3.01 (4H, m), 3.17-3.25 (1H, m), 3.51-3.54 (3H, m), 3.61-3.70 (5H, m), 3.94-3.97 (1H, m), 4.41 (1H, d, J=10.4), 4.56 (1H, d, J=10), 6.10 (1H, s).

### Example 33. Synthesis of N-methyl-N-((4-(methylsulfonyl)-1,4-oxazepan-2-yl)methyl)-6-(2-azaspiro[5.5]undecan-2-yl)-2-(trifluoromethyl)pyrimidin-4-amine (Compound 35)

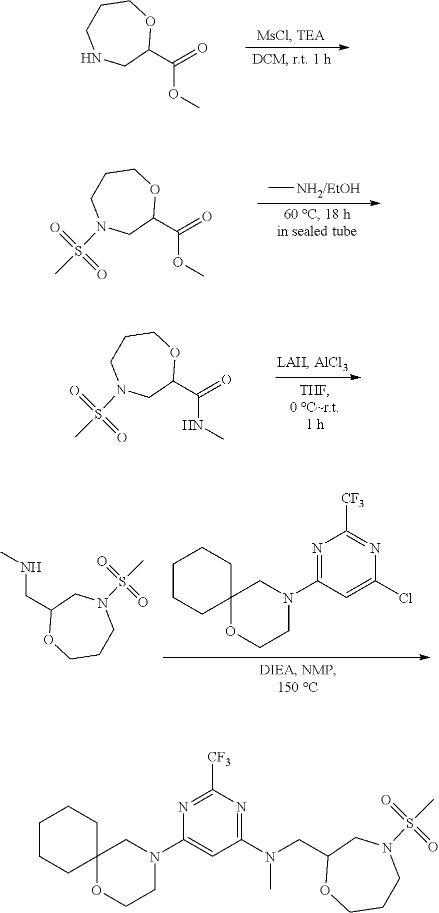

### Step A. Synthesis of methyl 4-(methylsulfonyl)-1,4-oxazepane-2-carboxylate

To a solution of methyl 1,4-oxazepane-2-carboxylate (250 mg, 1.57 mmol, 1.00 equiv), TEA (318 mg, 3.14 mmol, 2.00 equiv) in DCM (10 mL) was slowly added MsCl (179 mg, 1.57 mmol, 1.00 equiv) at r.t. The mixture was stirred at room temperature for 1 h. The mixture was extracted with DCM (10.00 mL×3) and water (10.00 mL). The organic layer was dried over Na₂SO₄ and concentrated to afford methyl 4-(methylsulfonyl)-1,4-oxazepane-2-carboxylate (559 mg, >100%) as yellow oil. LCMS: [M+H]⁺=238.

### Step B. Synthesis of N-methyl-4-(methylsulfonyl)-1,4-oxazepane-2-carboxamide

The mixture of 4-(methylsulfonyl)-1,4-oxazepane-2-carboxylate (559 mg, 1.57 mmol, 1.00 equiv) in the solution of methylamine in EtOH (10 mL) was stirred at 60 °C in sealed tube for 18 h. The mixture was concentrated in vacuum to afford N-methyl-4-(methylsulfonyl)-1,4-oxazepane-2-carboxamide (576 mg, >100%) as yellow solid. LCMS: [M+H]⁺=237.1.

### Step C. Synthesis of N-methyl-1-(4-(methylsulfonyl)-1,4-oxazepan-2-yl)methanamine

To a solution of N-methyl-4-(methylsulfonyl)-1,4-oxazepane-2-carboxamide (576 mg, 1.57 mmol, 1.00 equiv) in THF (10 mL) was added AlCl₃ (209 mg, 1.57 mmol, 1.00 equiv) and LAH (1M in THF, 4.71 mL, 4.71 mmol, 3.00 equiv) at 0 °C. The mixture was stirred at room temperature for 1 h. The mixture was quenched with the iced solution of NaHCO₃ and diluted with THF. The mixture was filtered and the filtrate was concentrated in vacuum to remove the solvent. The residue was washed with MeOH and purified by flash chromatography (DCM/MeOH/NH₃·H₂O=10/1/0.1) to afford N-methyl-1-(4-(methylsulfonyl)-1,4-oxazepan-2-yl)methanamine (404 mg, >100%) as yellow oil. LCMS: [M+H]⁺=223.1

### Step D. Synthesis of N-methyl-N-((4-(methylsulfonyl)-1,4-oxazepan-2-yl)methyl)-6-(2-azaspiro[5.5]undecan-2-yl)-2-(trifluoromethyl)pyrimidin-4-amine

To a solution of N-methyl-1-(4-(methylsulfonyl)-1,4-oxazepan-2-yl)methanamine (404 mg, 1.57 mmol, 1.00 equiv) in NMP (5 mL) was added DIEA (405 mg, 3.14 mmol, 2.00 equiv) and 2-(6-chloro-2-(trifluoromethyl)pyrimidin-4-yl)-2-azaspiro[5.5]undecane (523 mg, 1.57 mmol, 1.00 equiv) at r.t. The mixture was stirred at 140 °C for 3 h. The mixture was extracted with EA (10.00 mL×3) and water (10.00 mL). The organic layer was dried over Na₂SO₄ and concentrated in vacuum to remove the solvent. The residue was purified by Prep-HPLC with the following conditions: Column: XBridge C18 SN271/3611211208 Waters, 19×150 mm, 5 μm; Mobile Phase A: Water (0.1% NH₄HCO₃), Mobile Phase B: ACN; Flow rate: 15 mL/min; Gradient: 65% B to 95% B in 10 min. Wavelength: 214 nm. This afforded the title compound (78 mg, 10%) as a white solid. LCMS: [M+H]⁺=520.2. ¹HNMR (400 MHz, CDCl₃): δ 1.19-1.30 (2H, m), 1.30-1.67 (12H, m), 1.91-2.03 (2H, m), 2.81 (3H, s), 2.92-3.02 (1H, m), 3.04 (3H, s), 3.18-3.30 (1H, m), 3.44-3.57 (5H, m), 3.60-3.78 (3H, m), 3.81-3.92 (2H, m), 4.02-4.11 (1H, m), 5.43 (1H, s).

### Example 34. Synthesis of methyl 3-(((6-(2-azaspiro[5.5]undecan-2-yl)-2-(trifluoromethyl)pyrimidin-4-yl)(methyl)amino)methyl)piperidine-1-carboxylate (Compound 36)

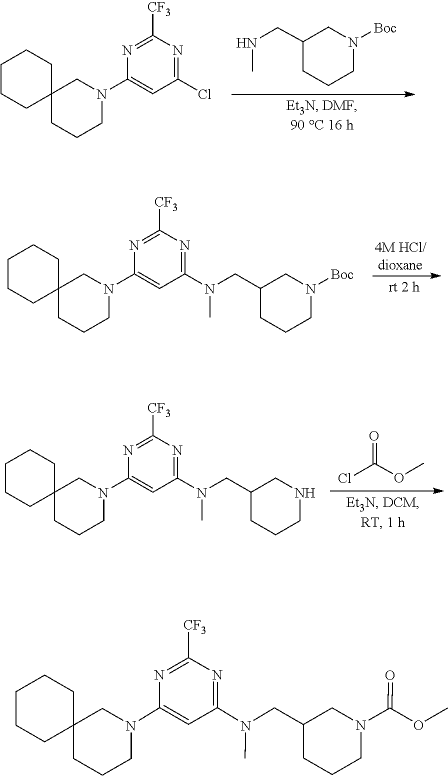

### Step A. Synthesis of tert-butyl 3-(((6-(2-azaspiro[5.5]undecan-2-yl)-2-(trifluoromethyl)pyrimidin-4-yl)(methyl)amino)methyl)piperidine-1-carboxylate

To a solution of 2-(6-chloro-2-(trifluoromethyl)pyrimidin-4-yl)-2-azaspiro[5.5]undecane (550 mg, 1.65 mmol, 1.00 equiv) in DMF (10 mL) was added tert-butyl 3-((methylamino)methyl)piperidine-1-carboxylate (452 mg, 1.98 mmol, 1.20 equiv) and Et₃N (1.14 mL, 8.25 mmol, 5.00 equiv) and heated to 90 °C. The reaction was stirred at 90 °C for 16 h. The reaction was diluted with EA and washed with water and brine. The organic layer was dried over anhydrous sodium sulfate, filtered and concentrated to obtain crude product. The crude product was purified by flash chromatography (EA/PE=5%) to afford tert-butyl 3-(((6-(2-azaspiro[5.5]undecan-2-yl)-2-(trifluoromethyl)pyrimidin-4-yl)(methyl)amino)methyl)piperidine-1-carboxylate (800 mg, 92%) as yellow gummy oil. LCMS: [M+H]⁺=526.

### Step B. Synthesis of N-methyl-N-(piperidin-3-ylmethyl)-6-(2-azaspiro[5.5]undecan-2-yl)-2-(trifluoromethyl)pyrimidin-4-amine

To a solution of tert-butyl 3-(((6-(2-azaspiro[5.5]undecan-2-yl)-2-(trifluoromethyl)pyrimidin-4-yl)(methyl)amino)methyl)piperidine-1-carboxylate (800 mg, 1.52 mmol, 1.00 equiv) in dioxane (8 mL) was added HCl/dioxane (4M, 8 mL) and stirred at r.t for 4 h. The mixture was adjust pH=7-8, then extracted with DCM, washed with water and brine, dried over Na₂SO₄, filtered and concentrated to get N-methyl-N-(piperidin-3-ylmethyl)-6-(2-azaspiro[5.5]undecan-2-yl)-2-(trifluoromethyl)pyrimidin-4-amine (527 mg, 81%) as yellow gummy oil, which was used to the next step without purification. LCMS: [M+H]⁺=426.

### Step C. Synthesis of methyl 3-(((6-(2-azaspiro[5.5]undecan-2-yl)-2-(trifluoromethyl)pyrimidin-4-yl)(methyl)amino)methyl)piperidine-1-carboxylate

To a solution of N-methyl-N-(piperidin-3-ylmethyl)-6-(2-azaspiro[5.5]undecan-2-yl)-2-(trifluoromethyl)pyrimidin-4-amine (90 mg, 0.21 mmol, 1.00 equiv) in DCM (2 mL) was added methyl carbonochloridate (30 mg, 0.32 mmol, 1.52 equiv) and Et₃N (0.15 mL, 1.05 mmol, 5.00 equiv) and heated to 25 °C. The reaction was stirred at 25 °C for 1 h. The reaction was diluted with DCM and washed with water and brine. The organic layer was dried over anhydrous sodium sulfate, filtered and concentrated to obtain crude product. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 60% B to 95% B in 10 min, Wavelength: 214 nm. This afforded the title compound (49 mg, 48%) as white solid. LCMS: [M+H]⁺=484.2. ¹HNMR (400 MHz, DMSO-d₆): δ 1.40-1.53 (16H, m), 1.62-1.66 (2H, m), 1.80-1.84 (1H, m), 2.63-2.68 (1H, m), 2.81-2.86 (1H, m), 2.99 (3H, s), 3.41-3.54 (9H, m), 3.75-3.78 (2H, m), 5.72 (1H, s).

### Example 35. Synthesis of N-benzyl-N-((4-(methylsulfonyl)morpholin-2-yl)methyl)-6-(2-azaspiro[5.5]undecan-2-yl)-2-(trifluoromethyl)pyrimidin-4-amine (Compound 37)

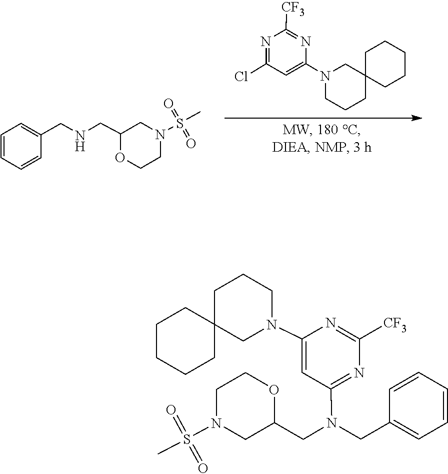

To a solution of N-benzyl-1-(4-(methylsulfonyl)morpholin-2-yl)methanamine (102 mg, 0.36 mmol, 1.00 equiv.) in NMP (2 mL) added 2-(6-chloro-2-(trifluoromethyl)pyrimidin-4-yl)-2-azaspiro[5.5]undecane (120 mg, 0.36 mmol, 1.00 equiv.) and DIEA (116 mg, 0.90 mmol, 2.50 equiv.). The mixture was irradiated in microwave at 180 °C for 3 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 70% B to 95% B in 10 min. Wavelength: 214 nm. This afforded the title compound (55 mg, 27%) as white solid. LCMS: [M+H]⁺=582.2. ¹H NMR (400 MHz, DMSO-d6): δ 1.02-1.24 (6H, m), 1.25-1.51 (10H, m), 2.58-2.66 (1H, m), 2.78-2.83 (1H, m), 2.86 (3H, s), 3.40-3.62 (6H, m), 3.74-3.77 (2H, m), 3.92-3.95 (1H, m), 4.67-4.88 (2H, m), 5.77 (1H, s), 7.20-7.26 (3H, m), 7.30-7.34 (2H, m).

### Example 36. Synthesis of N-ethyl-N-((4-(methylsulfonyl)morpholin-2-yl)methyl)-6-(2-azaspiro[5.5]undecan-2-yl)-2-(trifluoromethyl)pyrimidin-4-amine (Compound 38)

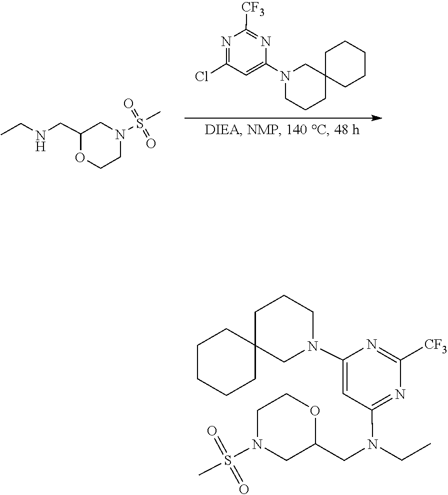

To a solution of N-((4-(methylsulfonyl)morpholin-2-yl)methyl)ethanamine (100 mg, 0.45 mmol, 1.00 equiv.) in NMP (2 mL) was added 2-(6-chloro-2-(trifluoromethyl)pyrimidin-4-yl)-2-azaspiro[5.5]undecane (150 mg, 0.45 mmol, 1.00 equiv.) and DIEA (145 mg, 1.12 mmol, 2.50 equiv.). The mixture was stirred at 140 °C for 48 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 5% B to 45% B in 10 min. Wavelength: 214 nm. This afforded the title compound (20 mg, 9%) as white solid. LCMS: [M+H]⁺=520.2. ¹H NMR (400 MHz, DMSO-d6): δ 1.07 (3H, t, J=6.8 Hz), 1.14-1.19 (2H, m), 1.30-1.35 (4H, m), 1.40-1.54 (9H, m), 2.57-2.63 (1H, m), 2.78-2.82 (1H, m), 2.86 (3H, s), 3.40-3.60 (9H, m), 3.63-3.75 (2H, m), 3.93 (1H, m), 5.80 (1H, s).

### Example 37. Synthesis of N1-(6-(2-azaspiro[5.5]undecan-2-yl)-2-(trifluoromethyl)pyrimidin-4-yl)-N2,N2-dimethyl-N1-((4-(methylsulfonyl)morpholin-2-yl)methyl)ethane-1,2-diamine (Compound 39)

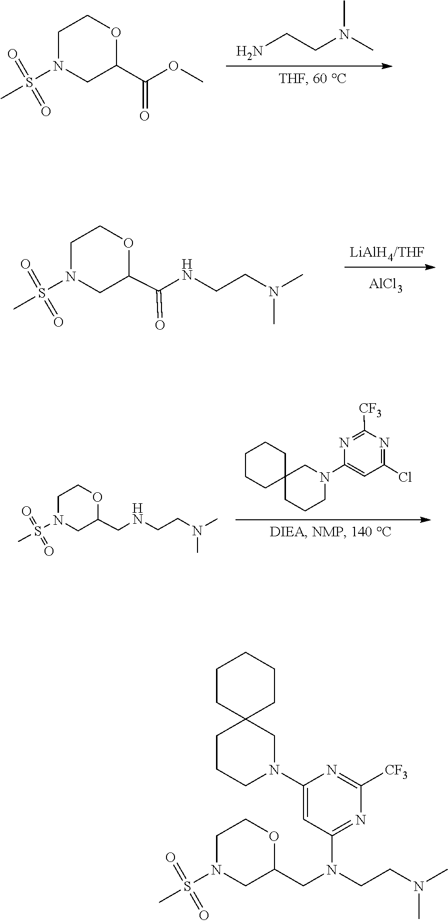

### Step A. Synthesis of N-(2-(dimethylamino)ethyl)-4-(methylsulfonyl)morpholine-2-carboxamide

To a solution of methyl 4-(methylsulfonyl)morpholine-2-carboxylate (150 mg, 0.67 mmol, 1.0 eq.) in THF (6 mL) stirred under nitrogen at 25 °C was added N1,N1-dimethylethane-1,2-diamine (178 mg, 2.01 mmol, 3.0 eq.). The reaction mixture was stirred at 60 °C for 16 h in seal tube. The mixture was concentrated to afford N-(2-(dimethylamino)ethyl)-4-(methylsulfonyl)morpholine-2-carboxamide (80 mg, 43%) as white solid. LCMS: [M+H]⁺=280.0.

### Step B. Synthesis of N1,N1-dimethyl-N2-((4-(methylsulfonyl)morpholin-2-yl)methyl)ethane-1,2-diamine

To a solution of AlCl₃ (38 mg, 0.28 mmol, 1.0 eq.) in THF (2 mL) stirred under nitrogen was added LiAlH₄ (33 mg, 0.86 mmol, 3.0 eq.) at 0 °C dropwise. The reaction mixture was stirred at 0 °C for 0.5 h. Then a solution of N-(2-(dimethylamino)ethyl)-4-(methylsulfonyl)morpholine-2-carboxamide (80 mg, 0.29 mmol, 1.0 eq.) in THF (4 mL) was added. The reaction mixture was stirred at 25 °C for 2 h. The mixture was quenched with Na₂SO₄ aq. (5 mL) at 0 °C. Then filtered and concentrated to get crude of N-(2-(dimethylamino)ethyl)-4-(methylsulfonyl)morpholine-2-carboxamide (60 mg, 79%) as colorless oil. LCMS: [M+H]⁺=266.4.

### Step C. Synthesis of N1-(6-(2-azaspiro[5.5]undecan-2-yl)-2-(trifluoromethyl)pyrimidin-4-yl)-N2,N2-dimethyl-N1-((4-(methylsulfonyl)morpholin-2-yl)methyl)ethane-1,2-diamine

To a solution of N-(2-(dimethylamino)ethyl)-4-(methylsulfonyl)morpholine-2-carboxamide (60 mg, 0.23 mmol, 1.00 eq.) and DIEA (44 mg, 0.34 mmol, 1.50 eq.) in NMP (2 mL) at 25 °C was added 2-(6-chloro-2-(trifluoromethyl)pyrimidin-4-yl)-2-azaspiro[5.5]undecane (75 mg, 0.23 mmol, 1.00 eq.). The reaction mixture was stirred at 140 °C for 16 h. The mixture was purified by flash chromatography (DCM/MeOH=10/1) to get crude. The residue was purified by using Prep-HPLC with the following conditions: Column: Sunfire Prep C18 Column, 19×150 mm, 5 μm; Mobile Phase A: Water (0.2% HCOOH), Mobile Phase B: ACN; Flow rate: 15 mL/min; Gradient: 25% B to 45% B in 10 min. Wavelength: 214 nm. This afforded the title compound (3.50 mg, 3%) as light-yellow solid. LCMS: [M+H]⁺=563.3. H NMR (400 MHz, CD₃OD): 1.22-1.27 (2H, m), 1.35-1.56 (10H, m), 1.59-1.63 (2H, m), 2.36 (6H, s), 2.60-2.67 (3H, m), 2.83 (3H, s), 2.84-2.91 (1H, m), 3.45-3.80 (12H, m), 3.97-4.01 (1H, m), 5.74 (1H, s).

### Example 38. Synthesis of 6-(9,9-difluoro-2-azaspiro[5.5]undecan-2-yl)-N-methyl-N-((4-(methylsulfonyl)morpholin-2-yl)methyl)-2-(trifluoromethyl)pyrimidin-4-amine (Compound 40)

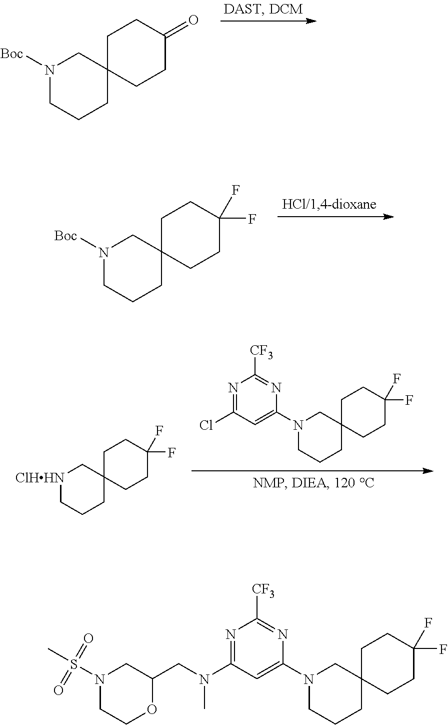

### Step A. Synthesis of tert-butyl 9,9-difluoro-2-azaspiro[5.5]undecane-2-carboxylate

To a solution of tert-butyl 9-oxo-2-azaspiro[5.5]undecane-2-carboxylate (200 mg, 0.75 mmol, 1.00 equiv) in DCM (4 mL) were added DSTA (361 mg, 2.24 mmol, 3.00 equiv) at 0 °C. The mixture was stirred at 25 °C for 16 h. The mixture was diluted with ice/Na₂CO₃ aq (10 mL), extracted with DCM (3×20 mL). The combined organic layers were washed with brine and concentrated in vacuo. The crude was purified by flash chromatography (PE/EA=15/1) to afford tert-butyl 9,9-difluoro-2-azaspiro[5.5]undecane-2-carboxylate (130 mg, 60%) as a colorless oil. LCMS: [M+H]⁺=290.

### Step B. Synthesis of 9,9-difluoro-2-azaspiro[5.5]undecane hydrochloride

To a solution of 9,9-difluoro-2-azaspiro[5.5]undecane-2-carboxylate (110 mg, 0.38 mmol, 1.00 equiv) in 1,4-dioxane (1 mL) was added HCl/1,4-dioxane (4M, 4 mL). The mixture was stirred at room temperature for 2 h. The mixture was concentrated to get the crude 9,9-difluoro-2-azaspiro[5.5]undecane hydrochloride (HCl salt) (115 mg, >95%) as off-white solid. LCMS: [M+H]⁺=190.

### Step C. Synthesis of 6-(9,9-difluoro-2-azaspiro[5.5]undecan-2-yl)-N-methyl-N-((4-(methylsulfonyl)morpholin-2-yl)methyl)-2-(trifluoromethyl)pyrimidin-4-amine

To a stirred solution of 9,9-difluoro-2-azaspiro[5.5]undecane hydrochloride (HCl salt) (115 mg, 0.51 mmol, 1.65 equiv) and 2-(6-chloro-2-(trifluoromethyl)pyrimidin-4-yl)-9,9-difluoro-2-azaspiro[5.5]undecane (121 mg, 0.31 mmol, 1.00 equiv) in NMP (3 mL) was added DIEA (121 mg, 0.93 mmol, 3.00 equiv). The resulting mixture was stirred at 120 °C for 4 h. The mixture was concentrated under reduced pressure. The resulted solution was purified using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 30% B to 60% B in 12 min; Wavelength: 214 nm. This afforded the title compound (28 mg, 10%) as a white solid. LCMS: [M+H]⁺=542. ¹H NMR (400 MHz, DMSO-d6): δ 1.32-1.39 (2H, m), 1.49-1.54 (6H, m), 1.99-2.03 (2H, m), 1.85-1.95 (2H, m), 2.59-2.61 (1H, m), 2.79-2.86 (4H, m), 3.05 (3H, s), 3.32-3.40 (2H, m), 3.47-3.70 (6H, m), 3.72-3.92 (2H, m), 3.94 (1H, m), 5.83 (1H, s).

### Example 39. Synthesis of N-((4,4-difluoro-1-(methylsulfonyl)piperidin-3-yl)methyl)-N-methyl-6-(2-azaspiro[5.5]undecan-2-yl)-2-(trifluoromethyl)pyrimidin-4-amine (Compound 41)

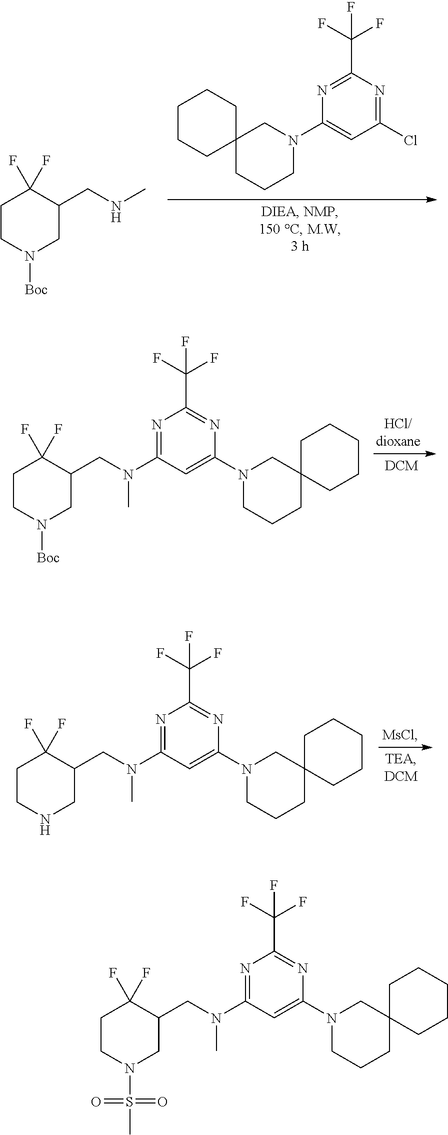

### Step A. Synthesis of tert-butyl 3-(((6-(2-azaspiro[5.5]undecan-2-yl)-2-(trifluoromethyl)pyrimidin-4-yl)(methyl)amino)methyl)-4,4-difluoropiperidine-1-carboxylate

To a stirred solution of 2-(6-chloro-2-(trifluoromethyl)pyrimidin-4-yl)-2-azaspiro[5.5]undecane (70 mg, 0.21 mmol, 1.00 equiv) in NMP (3 mL) was added DIEA (82 mg, 0.63 mmol, 3.00 equiv) and tert-butyl 4,4-difluoro-3-((methylamino)methyl)piperidine-1-carboxylate (crude 65.00 mg, 0.24 mmol, 1.20 equiv). The resulting mixture was stirred at 150 °C for 2 h in a microwave. The mixture was added water (20 mL), extracted with EtOAc (10 mL). The combined organic layer was washed with brine and concentrated in vacuo. The residue was purified by flash chromatography (EA/PE=6%) to afford tert-butyl 3-(((6-(2-azaspiro[5.5]undecan-2-yl)-2-(trifluoromethyl)pyrimidin-4-yl)(methyl)amino)methyl)-4,4-difluoropiperidine-1-carboxylate (99 mg, 84%) as a yellow oil. LCMS: [M+H]=562.

### Step B. Synthesis of N-((4,4-difluoropiperidin-3-yl)methyl)-N-methyl-6-(2-azaspiro[5.5]undecan-2-yl)-2-(trifluoromethyl)pyrimidin-4-amine hydrochloride

To a solution of tert-butyl 3-(((6-(2-azaspiro[5.5]undecan-2-yl)-2-(trifluoromethyl)pyrimidin-4-yl)(methyl)amino)methyl)-4,4-difluoropiperidine-1-carboxylate (99 mg, 0.17 mmol, 1.00 equiv) in DCM (4 mL) was added 4M HCl/dioxane (1 mL). The mixture was stirred at R.T. for 1 h. The mixture was concentrated in vacuo to get the crude N-((4,4-difluoropiperidin-3-yl)methyl)-N-methyl-6-(2-azaspiro[5.5]undecan-2-yl)-2-(trifluoromethyl)pyrimidin-4-amine hydrochloride (160 mg, >100%) as yellow oil. LCMS: [M+H]⁺=464.

### Step C. Synthesis of N-((4,4-difluoro-1-(methylsulfonyl)piperidin-3-yl)methyl)-N-methyl-6-(2-azaspiro[5.5]undecan-2-yl)-2-(trifluoromethyl)pyrimidin-4-amine

To a solution of N-((4,4-difluoropiperidin-3-yl)methyl)-N-methyl-6-(2-azaspiro[5.5]undecan-2-yl)-2-(trifluoromethyl)pyrimidin-4-amine hydrochloride (crude 160 mg, 0.17 mmol, 1.00 equiv) in DCM (4 mL) was added TEA (54 mg, 0.53 mmol, 3.00 equiv) and MsCl (30 mg, 0.26 mmol, 1.50 equiv). The mixture was stirred at R.T. for 0.5 h. The mixture was added water, extracted with DCM. The combined organic was concentrated in vacuo. The residue was purified using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 90% B to 90% B in 12 min; Wavelength: 214 nm. This afforded the title compound (18 mg, 20%) as a white solid. LCMS: [M+H]⁺=540.2. ¹HNMR (400 MHz, CDCl₃-d): δ 1.27-1.64 (15H, m), 2.03-2.22 (2H, m), 2.40-2.47 (1H, m), 2.76 (3H, s), 2.95-3.08 (5H, m), 3.43-3.66 (5H, m), 3.67-3.76 (2H, m), 4.11-4.17 (1H, m), 5.43 (1H, s).

### Example 40. Synthesis of 2-(((6-(2-azaspiro[5.5]undecan-2-yl)-2-(trifluoromethyl)pyrimidin-4-yl)(methyl)amino)methyl)-4-(methylsulfonyl)thiomorpholine 1,1-dioxide (Compound 42)

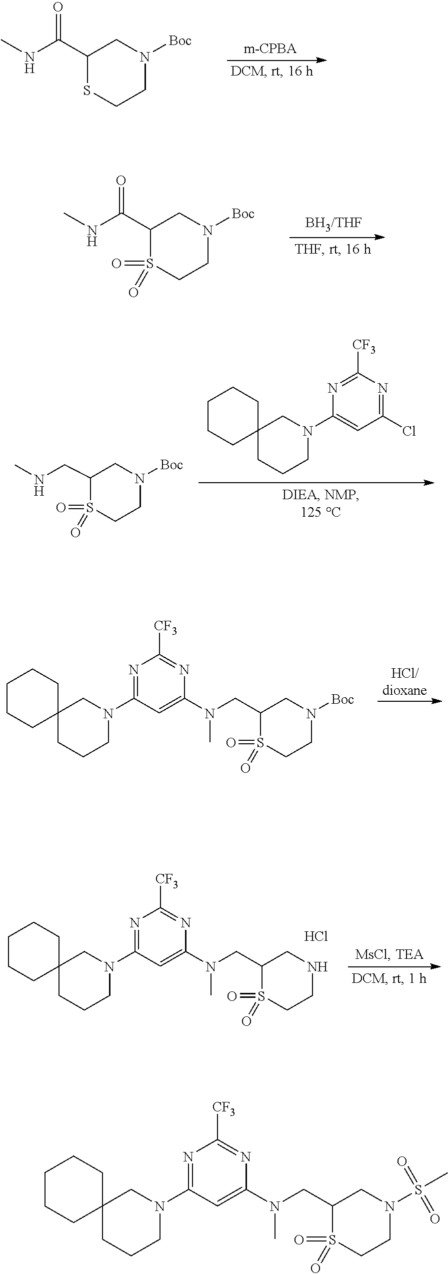

### Step A. Synthesis of tert-butyl 2-(methylcarbamoyl)thiomorpholine-4-carboxylate 1,1-dioxide

To a solution of tert-butyl 2-((methylamino)methyl)thiomorpholine-4-carboxylate (290 mg, 1.12 mmol, 1.00 equiv) DCM (10 mL) was added m-CPBA (581 mg, 3.36 mmol, 3.00 equiv) at 0 °C. The mixture was stirred at room temperature for 16 h. The residue was extracted with DCM and washed with saturated NaHCO₃. The organic layer was dried over Na₂SO₄ and concentrated. The residue was purified by column eluted with PE/EA=1/1 to get the tert-butyl 2-(methylcarbamoyl)thiomorpholine-4-carboxylate 1,1-dioxide (214 mg, 66%) as white solid. LCMS: [M+H-56]⁺=237

### Step B. Synthesis of tert-butyl 2-((methylamino)methyl)thiomorpholine-4-carboxylate 1,1-dioxide

To a solution of 2-(methylcarbamoyl)thiomorpholine-4-carboxylate 1,1-dioxide (280 mg, 0.96 mmol, 1.00 equiv) in THF (5 mL) was added 4.80 mL BH₃/THF (3N borane in THF, 5.00 equiv). The mixture was stirred at room temperature for 16 h. The mixture was added 5 mL MeOH and concentrated to get crude tert-butyl 2-((methylamino)methyl)thiomorpholine-4-carboxylate 1,1-dioxide (320 mg, >100%) as colorless oil. LCMS: [M+H]⁺=279

### Step C. Synthesis of tert-butyl 2-(((6-(2-azaspiro[5.5]undecan-2-yl)-2-(trifluoromethyl)pyrimidin-4-yl)(methyl)amino)methyl)thiomorpholine-4-carboxylate 1,1-dioxide

To a solution of 2-(6-chloro-2-(trifluoromethyl)pyrimidin-4-yl)-2-azaspiro[5.5]undecane (72 mg, 0.22 mmol, 1.00 equiv) and tert-butyl 2-((methylamino)methyl)thiomorpholine-4-carboxylate 1,1-dioxide (90 mg, 0.32 mmol, 1.5 equiv) in NMP (5 mL) was added DIEA (84 mg, 0.65 mmol, 3.0 equiv). The mixture was stirred at 125 °C for 16 h. The mixture was extracted with EtOAc and washed with brine. The organic layer was dried over Na₂SO₄ and concentrated. The residue was purified by column eluted with PE/EA=4/1 to get the tert-butyl 2-(((6-(2-azaspiro[5.5]undecan-2-yl)-2-(trifluoromethyl)pyrimidin-4-yl)(methyl)amino)methyl)thiomorpholine-4-carboxylate 1,1-dioxide (56 mg, 45%) as yellow oil. LCMS: [M+H]⁺=576.3.

### Step D. Synthesis of 2-(((6-(2-azaspiro[5.5]undecan-2-yl)-2-(trifluoromethyl)pyrimidin-4-yl)(methyl)amino)methyl)thiomorpholine 1,1-dioxide (HCl Salt)

To a solution of tert-butyl 2-(((6-(2-azaspiro[5.5]undecan-2-yl)-2-(trifluoromethyl)pyrimidin-4-yl)(methyl)amino)methyl)thiomorpholine-4-carboxylate 1,1-dioxide (55 mg, 0.10 mmol, 1.00 equiv) in DCM (2 mL) was added 1 mL HCl (4N in 1,4-dioxane). The mixture was stirred at room temperature for 2 h. The mixture was concentrated to get crude 2-(((6-(2-azaspiro[5.5]undecan-2-yl)-2-(trifluoromethyl)pyrimidin-4-yl)(methyl)amino)methyl)thiomorpholine 1,1-dioxide (HCl salt) (57 mg, >100%) as colorless oil. LCMS: [M+H]⁺=476.2.

### Step E. Synthesis of 2-(((6-(2-azaspiro[5.5]undecan-2-yl)-2-(trifluoromethyl)pyrimidin-4-yl)(methyl)amino)methyl)-4-(methylsulfonyl)thiomorpholine 1,1-dioxide

To a solution of 2-(((6-(2-azaspiro[5.5]undecan-2-yl)-2-(trifluoromethyl)pyrimidin-4-yl)(methyl)amino)methyl)thiomorpholine 1,1-dioxide (HCl salt) (57 mg, 0.11 mmol, 1.00 equiv) and TEA (43 mg, 0.43 mmol, 4.00 equiv) in DCM (4 mL) was added MsCl (25 mg, 0.22 mmol, 1.50 equiv). The mixture was stirred at room temperature for 1 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 70% B to 90% B in 8 min, Wavelength: 214 nm. This afforded the title compound (27 mg, 44%) as white solid. LMCS: [M+H]⁺=554.2. ¹H NMR (400 MHz, CDCl₃): δ 1.22-1.27 (2H, m), 1.36-1.51 (10H, m), 1.57-1.63 (2H, m), 2.88 (3H, s), 3.07 (3H, s), 3.15-3.20 (2H, m), 3.43-3.58 (7H, m), 3.73-3.78 (1H, m), 4.08-4.16 (2H, m), 4.24-4.30 (1H, m), 5.48 (1H, s).

### Example 41. Synthesis of N-methyl-N-((4-(methylsulfonyl)thiomorpholin-2-yl)methyl)-6-(2-azaspiro[5.5]undecan-2-yl)-2-(trifluoromethyl)pyrimidin-4-amine (Compound 43)

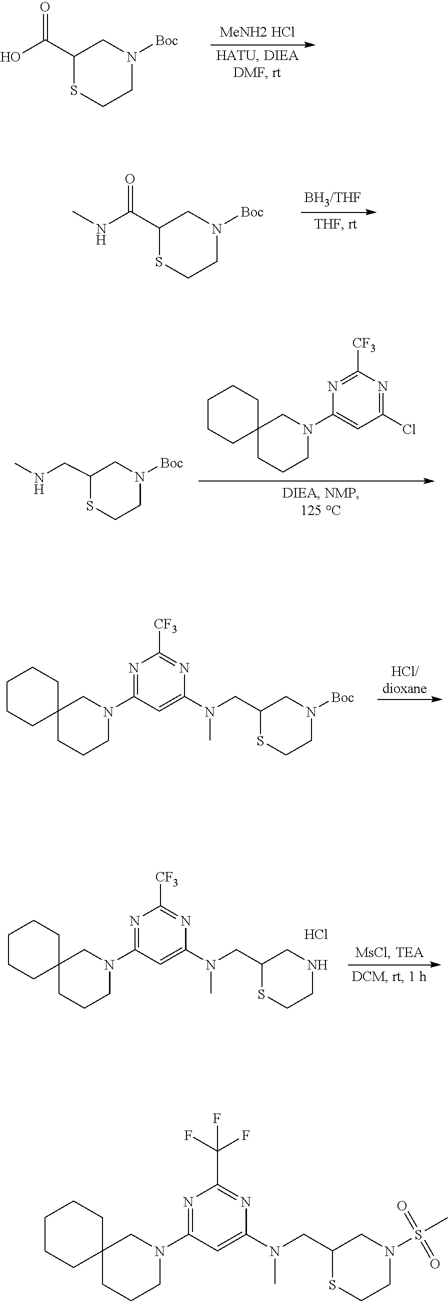

### Step A. Synthesis of tert-butyl 2-(methylcarbamoyl)thiomorpholine-4-carboxylate

To a solution of 4-(tert-butoxycarbonyl)thiomorpholine-2-carboxylic acid (1.00 g, 4.05 mmol, 1.00 equiv), and methylamine hydrochloride (407 mg, 6.07 mmol, 1.50 equiv), EDCI (1.17 g, 6.07 mmol, 1.50 equiv) and HOBt (820 mg, 6.07 mmol, 1.50 equiv) in DMF (15 mL) was added DIEA (1.6 mg, 12.10 mmol, 3.00 equiv). The mixture was stirred at room temperature for 16 h. The mixture was extracted with EtOAc and washed with water. The organic layer was dried over Na₂SO₄ and concentrated. The residue was purified by column eluted with PE/EA=1/1 to get the tert-butyl 2-(methylcarbamoyl)thiomorpholine-4-carboxylate (746 mg, 71%) as colorless oil. LCMS: [M+H-56]⁺=205.

### Step B. Synthesis of tert-butyl 2-((methylamino)methyl)thiomorpholine-4-carboxylate

To a solution of tert-butyl 2-(methylcarbamoyl)thiomorpholine-4-carboxylate (425 mg, 1.63 mmol, 1.00 equiv) in THF (5 mL) was added BH₃/THF (6.5 mL, IN borane in THF, 4.00 equiv). The mixture was stirred at room temperature for 6 h. The mixture was added 5 mL MeOH and concentrated to get the crude tert-butyl 2-((methylamino)methyl)thiomorpholine-4-carboxylate (510 mg, >100%) as colorless oil. LCMS: [M+H]⁺=247.

### Step C. Synthesis of tert-butyl 2-(((6-(2-azaspiro[5.5]undecan-2-yl)-2-(trifluoromethyl)pyrimidin-4-yl)(methyl)amino)methyl)thiomorpholine-4-carboxylate

To a solution of 2-(6-chloro-2-(trifluoromethyl)pyrimidin-4-yl)-2-azaspiro[5.5]undecane (200 mg, 0.60 mmol, 1.00 equiv) and tert-butyl 2-((methylamino)methyl)thiomorpholine-4-carboxylate (296 mg, 1.20 mmol, 2.00 equiv) in NMP (5 mL) was added DIEA (310 mg, 2.40 mmol, 4.00 equiv). The mixture was stirred at 125 °C for 12 h. The mixture was extracted with EtOAc and washed with brine. The organic layer was dried over Na₂SO₄ and concentrated. The residue was purified by column eluted with PE/EA=10/1 to get the tert-butyl 2-(((6-(2-azaspiro[5.5]undecan-2-yl)-2-(trifluoromethyl)pyrimidin-4-yl)(methyl)amino)methyl)thiomorpholine-4-carboxylate (212 mg, 65%) as yellow solid. LCMS: [M+H]⁺=544.3.

### Step D. Synthesis of N-methyl-6-(2-azaspiro[5.5]undecan-2-yl)-N-(thiomorpholin-2-ylmethyl)-2-(trifluoromethyl)pyrimidin-4-amine (HCl Salt)

To a solution of tert-butyl 2-(((6-(2-azaspiro[5.5]undecan-2-yl)-2-(trifluoromethyl)pyrimidin-4-yl)(methyl)amino)methyl)thiomorpholine-4-carboxylate (50 mg, 0.09 mmol, 1.00 equiv) in DCM (2 mL) was added 1 mL HCl (4 N in 1,4-dioxane). The mixture was stirred at room temperature for 2 h. The mixture was concentrated to get the crude N-methyl-6-(2-azaspiro[5.5]undecan-2-yl)-N-(thiomorpholin-2-ylmethyl)-2-(trifluoromethyl)pyrimidin-4-amine (HCl salt) (52 mg, >100%) as off-white solid. LCMS: [M+H]⁺=444.3.

### Step E. Synthesis of N-methyl-N-((4-(methylsulfonyl)thiomorpholin-2-yl)methyl)-6-(2-azaspiro[5.5]undecan-2-yl)-2-(trifluoromethyl)pyrimidin-4-amine

To a solution of N-methyl-6-(2-azaspiro[5.5]undecan-2-yl)-N-(thiomorpholin-2-ylmethyl)-2-(trifluoromethyl)pyrimidin-4-amine (HCl salt) (52 mg, 0.11 mmol, 1.00 equiv) and TEA (43 mg, 0.43 mmol, 4.00 equiv) in DCM (4 mL) was added MsCl (25 mg, 0.22 mmol, 2.00 equiv). The mixture was stirred at room temperature for 1 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 40% B to 95% B in 9 min, Wavelength: 214 nm. This afforded the title compound (25 mg, 45%) as white solid. LCMS: [M+H]⁺=522.2. ¹H NMR (400 MHz, CDCl₃): δ 1.22-1.27 (2H, m), 1.37-1.56 (10H, m), 1.60-1.63 (2H, m), 2.72-2.77 (4H, m), 2.82-2.88 (1H, m), 3.05 (3H, s), 3.23-3.38 (3H, m), 3.47-3.54 (4H, m), 3.62-3.70 (3H, m), 3.89-3.95 (1H, m), 5.43 (1H, s).

### Example 42. Synthesis of N-methyl-N-(2-(4-(methylsulfonyl)morpholin-2-yl)ethyl)-6-(2-azaspiro[5.5]undecan-2-yl)-2-(trifluoromethyl)pyrimidin-4-amine (Compound 44)

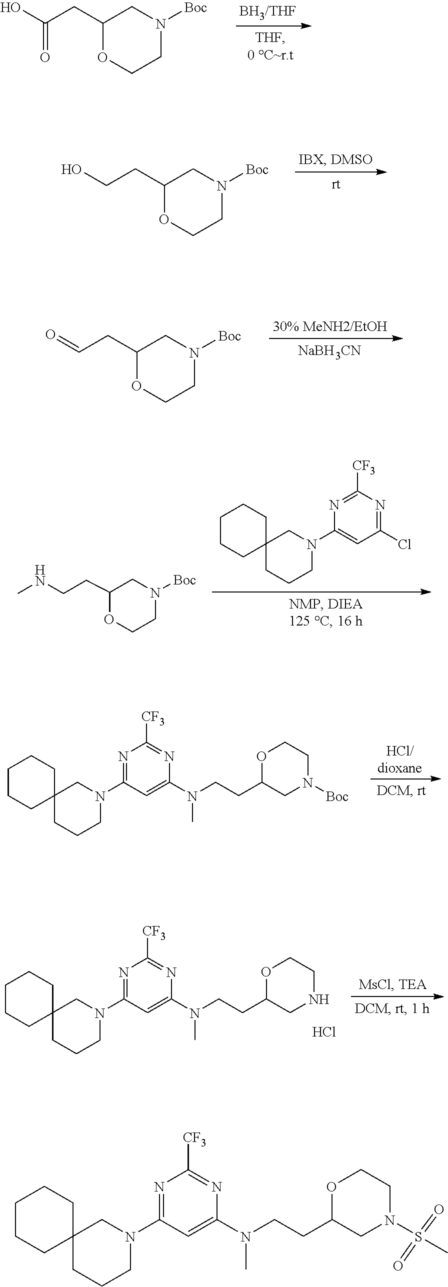

### Step A. Synthesis of tert-butyl 2-(2-hydroxyethyl)morpholine-4-carboxylate

To a solution of 2-(4-(tert-butoxycarbonyl)morpholin-2-yl)acetic acid (500 mg, 2.04 mmol, 1.00 equiv) in THF (10 mL) was added BH₃/THF (4.1 mL, 4.08 mmol, 2.00 equiv, 1M in THF) at 0 °C. The mixture was stirred at room temperature for 16 h. The mixture was diluted with MeOH, and stirred at room temperature for 2 h. Then the solvent was removed in vacuo. The residue was added DCM, filtered and concentrated, dried to get the crude product tert-butyl 2-(2-hydroxyethyl)morpholine-4-carboxylate (533 mg, >100%) as colorless oil, which was used directly in the next step without further purification. LCMS: [M+H-56]⁺=176.

### Step B. Synthesis of tert-butyl 2-(2-oxoethyl)morpholine-4-carboxylate

To a solution of tert-butyl 2-(2-hydroxyethyl)morpholine-4-carboxylate (70 mg, 0.30 mmol, 1.00 equiv) DMSO (2 mL) was added IBX (342 mg, 0.61 mmol, 2.00 equiv). The mixture was stirred at room temperature for 3 h. The mixture was extracted with EtOAc and washed with water. The organic layer was dried over Na₂SO₄ and concentrated to afford crude tert-butyl 2-(2-oxoethyl)morpholine-4-carboxylate (64 mg, 94%) as colorless oil.

### Step C. Synthesis of tert-butyl 2-(2-(methylamino)ethyl)morpholine-4-carboxylate

To a solution of tert-butyl 2-(2-oxoethyl)morpholine-4-carboxylate (64 mg, 0.28 mmol, 1.00 equiv) and methanamine (30% by weight in EtOH) (58 mg, 0.56 mmol, 2.00 equiv) in MeOH (5 mL) was added NaBH₃CN (35 mg, 0.56 mmol, 2.00 equiv). The mixture was stirred at room temperature for 2 h. The mixture was concentrated to afford crude tert-butyl 2-(2-(methylamino)ethyl)morpholine-4-carboxylate (110 mg, >100%) as colorless oil. LCMS: [M+H]⁺=245.

### Step D. Synthesis of tert-butyl 2-(2-((6-(2-azaspiro[5.5]undecan-2-yl)-2-(trifluoromethyl)pyrimidin-4-yl)(methyl)amino)ethyl)morpholine-4-carboxylate

To a solution of 2-(6-chloro-2-(trifluoromethyl)pyrimidin-4-yl)-2-azaspiro[5.5]undecane (60 mg, 0.18 mmol, 1.00 equiv) and 2-(2-(methylamino)ethyl)morpholine-4-carboxylate (66 mg, 0.27 mmol, 1.50 equiv) in NMP (2 mL) was added DIEA (46 mg, 0.36 mmol, 2.00 equiv). The mixture was stirred at 125 °C for 16 h. The mixture was extracted with EtOAc and washed with brine. The organic layer was dried over Na₂SO₄ and concentrated. The residue was purified by column eluted with PE/EA=5/1 to afford tert-butyl 2-(2-((6-(2-azaspiro[5.5]undecan-2-yl)-2-(trifluoromethyl)pyrimidin-4-yl)(methyl)amino)ethyl)morpholine-4-carboxylate (52 mg, 54%) as colorless oil. LCMS: [M+H]⁺=542.

### Step E. Synthesis of N-methyl-N-(2-(morpholin-2-yl)ethyl)-6-(2-azaspiro[5.5]undecan-2-yl)-2-(trifluoromethyl)pyrimidin-4-amine (HCl Salt)

To a solution of tert-butyl 2-(2-((6-(2-azaspiro[5.5]undecan-2-yl)-2-(trifluoromethyl)pyrimidin-4-yl)(methyl)amino)ethyl)morpholine-4-carboxylate (52 mg, 0.096 mmol, 1.00 equiv) in DCM (3 mL) was added 1 mL HCl (4N in 1,4-dioxane). The mixture was stirred at room temperature for 1 h. The mixture was concentrated to afford crude N-methyl-N-(2-(morpholin-2-yl)ethyl)-6-(2-azaspiro[5.5]undecan-2-yl)-2-(trifluoromethyl)pyrimidin-4-amine (HCl salt) (47 mg, >100%) as colorless oil. LCMS: [M+H]⁺=442.

### Step F. Synthesis of N-methyl-N-(2-(4-(methylsulfonyl)morpholin-2-yl)ethyl)-6-(2-azaspiro[5.5]undecan-2-yl)-2-(trifluoromethyl)pyrimidin-4-amine

To a solution of N-methyl-N-(2-(morpholin-2-yl)ethyl)-6-(2-azaspiro[5.5]undecan-2-yl)-2-(trifluoromethyl)pyrimidin-4-amine (HCl salt) (47 mg, 0.10 mmol, 1.00 equiv) and TEA (20 mg, 0.20 mmol, 2.00 equiv) in DCM (5 mL) was added MsCl (23 mg, 0.20 mmol, 2.00 equiv). The mixture was stirred at room temperature for 1 h. The mixture was concentrated. The residue was purified by using Prep-HPLC with the following conditions: Column: XBridge Prep C18 Column, 19×150 mm, 5 μm; Mobile Phase A: Water (0.1% NH₄HCO₃), Mobile Phase B: ACN; Flow rate: 15 mL/min; Gradient: 50% B to 95% B in 10 min, Wavelength: 214 nm. This afforded the title compound (30 mg, 58%) as white solid. LCMS: [M+H]⁺=520.2. ¹H NMR (400 MHz, CDCl₃): δ 1.21-1.26 (2H, m), 1.36-1.50 (10H, m), 1.61-1.72 (3H, m), 1.79-1.84 (1H, m), 2.53-2.60 (1H, m), 2.77-2.85 (4H, m), 3.01 (3H, s), 3.43-3.56 (7H, m), 3.62-3.73 (3H, m), 3.96-3.99 (1H, m), 5.43 (1H, s).

### Example 43. Screening of Compounds for Inhibitory Activity Against Acetyl-CoA Carboxylase (ACC)

Compounds of the disclosure were assayed for relative inhibition of ACC (C. elegans and human). The assay was adapted from that reported by Harriman et al., “Acetyl-CoA carboxylase inhibition by ND-630 reduces hepatic steatosis, improves insulin sensitivity, and modulates dyslipidemia in rats” PNAS E1796-E1805, Published online Mar. 14, 2016. The assay response signal was measured by luminescence after 45 minutes of reaction at 25 °C in a final reaction volume of 41 uL in white 384-well polypropylene flat-bottom plates. Initially, 10 mM DMSO stock solutions of each test compound were diluted to 41× concentration of the final assay concentration and 1 uL was transferred to the assay plate. To each well was added a 20 μL aliquot of Buffer A1 (50 mM Tris-HCl, pH 7.6, 400 μM ATP (disodium salt), 30 mM NaHCO₃, 2 mM DTT, 3 mM sodium citrate, 2 mM MgCl₂, and 6 nM human ACCase) for the human enzyme assay, or Buffer A2 (50 mM Tris-HCl pH 7.6, 400 μM ATP (disodium salt), 30 mM NaHCO₃, 2 mM DTT, 3 mM sodium citrate, 2 mM MgCl₂, 100 mM KCl, 50 mM NaCl, and 8 nM C. elegans ACC) for the C. elegans enzyme assay and the plate was then centrifuged at 1000 rpm for 1 minute. Next, reaction was initiated by adding to the assay plate wells 20 μL of Buffer B1 (50 mM Tris-HCl pH 7.6, 400 μM Ac-CoA, 30 mM NaHCO₃, 2 mM DTT, 3 mM sodium citrate, and 2 mM MgCl₂ for the human enzyme assay) or Buffer B2 (50 mM Tris pH 7.6, 400 μM Ac-CoA, 30 mM NaHCO₃, 2 mM DTT, 3 mM sodium citrate, 2 mM MgCl₂, 100 mM KCl, 50 mM NaCl for the C. elegans enzyme assay) and the plate was then centrifuged at 1000 rpm for 1 minute. The assay plate was sealed and incubated for 45 minutes at 25 °C. Afterwards, the assay plate was centrifuged at 1000 rpm for 1 minute. A 10 μL aliquot of the reaction mixture was then transferred to a white 384-well polypropylene flat-bottom plate containing 10 μL of ADP-Glo Reagent™ (Promega). This plate was sealed and incubated for 40 minutes at 25 °C. After incubation, the plate was centrifuged at 1000 rpm for 1 minute. ADP-Glo Detection Reagent™ (Promega; 20 uL) was added to each well to develop the luminescence signal and incubated at 25 °C for 30 minutes before being centrifuged for 1 minute at 1000 rpm and read on a BioTek Synergy HIM Multi Mode Reader in luminescence endpoint mode. The level of reaction inhibition by each test compound concentration was computed by averaging the uninhibited control wells (vehicle only) to normalize the maximum response. All other values are reported as a percentage of this maximum response value. All values, including the maximum response were also normalized to baseline by subtracting the average value of background control wells (containing no Ac-CoA substrate) from all wells. Fitting the computed percent inhibition at various concentrations of test compound to a 4-parameter sigmoidal function yielded the reported IC50 values. All IC50 values were determined from duplicate test compound concentrations in a single assay. Typical, between-assay experimental error was +/−2-fold.

Categorized results are provided in Table 1, which demonstrates that ACC IC50 values ranged from less than 500 nM to greater than 5 micromolar.

TABLE 1
ACCase Inhibition Activity

| | ACCase |
|---|---|
| Compound | Inhibition |
| # | Activity |
| 1 | 1 |
| 3 | 1 |
| 4 | 1 |
| 5 | 3 |
| 6 | 3 |
| 7 | 3 |
| 8 | 3 |
| 9 | 3 |
| 10 | 3 |
| 11 | 2 |
| 12 | 2 |
| 13 | 3 |
| 14 | 1 |
| 15 | 2 |
| 16 | 2 |
| 17 | 2 |
| 18 | 2 |
| 19 | 3 |
| 20 | 1 |
| 21 | 2 |
| 22 | 1 |
| 23 | 1 |
| 24 | 1 |
| 25 | 1 |
| 26 | 1 |
| 27 | 1 |
| 28 | 1 |
| 29 | 1 |
| 30 | 1 |
| 31 | 1 |
| 32 | 1 |
| 33 | 1 |
| 34 | 2 |
| 35 | 1 |
| 36 | 3 |
| 37 | 3 |
| 38 | 1 |
| 39 | 1 |
| 40 | 1 |
| 41 | 1 |
| 42 | 1 |
| 43 | 1 |
| 44 | 3 |

Categories:
1: IC50 ≤ 500 nM
2: IC50 = 500 to 5000 nM
3: IC50 ≥ 5 mM

### Example 44. Screening of Compounds Against Microfilaria of Dirofilaria immitis

To evaluate the efficacy of compounds of the disclosure against Dirofilaria immitis, L1 stage larvae were exposed to various concentrations of the compounds to determine survival or paralysis of the larvae. Results are reported as the minimum effective dose (MED), defined as the concentration required to eliminate motility.

To perform the assay, microfilaria of Dirofilaria immitis were added to the wells of a microtiter plate containing buffer and the test compounds in DMSO. Microfilaria exposed to DMSO alone served as controls. An assessment was conducted at 72 hours to determine survival of the microfilaria. Categorized results are provided in Table 2, which demonstrates that the minimum effective dose (“MED”) ranged from about 10 mM up to 100 mM or more.

Table 2. Minimum Effective Dose (MED) of Compounds of the Disclosure Screened Against Dirofilaria immitis L1 Larvae

TABLE 2
Minimum effective dose (MED) of compounds of the disclosure
screened against Dirofilaria immitis L1 larvae

| | Ranked D. |
|---|---|
| | immitis L1 MED |
| Compound # | Activity |
| 1 | 1 |
| 3 | 2 |
| 4 | 2 |
| 5 | 3 |
| 6 | 3 |
| 7 | 3 |
| 8 | 3 |
| 9 | 2 |
| 10 | 3 |
| 11 | 2 |
| 12 | 3 |
| 13 | 1 |
| 14 | 3 |
| 15 | 1 |
| 16 | 2 |
| 17 | 3 |
| 18 | 3 |
| 19 | 3 |
| 20 | 1 |
| 21 | 1 |
| 22 | 3 |
| 23 | 1 |
| 24 | 1 |
| 25 | 3 |
| 26 | 2 |
| 27 | 2 |
| 28 | 1 |
| 29 | 2 |
| 30 | 1 |
| 31 | 2 |
| 32 | 1 |
| 33 | 1 |
| 34 | 2 |
| 35 | 1 |
| 36 | 3 |
| 37 | 3 |
| 38 | 2 |
| 39 | 1 |
| 40 | 2 |
| 41 | 2 |
| 42 | 3 |
| 43 | 3 |
| 44 | 3 |

Categories:
1: ≤10 mM
2: ≤30 mM
3: ≥100 mM

What is claimed is:

1. A compound having a structure according to Formula I: 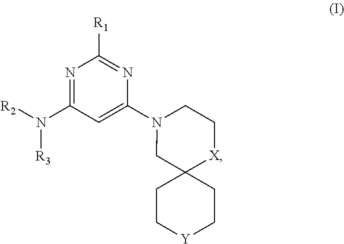
or a pharmaceutically acceptable salt, wherein:
R₁ is H or C₁-C₆ alkyl, wherein said C₁-C₆ alkyl is optionally substituted with one or more F atoms;
X is CH₂, O, S, or SO₂;
Y is a bond or is CR₄R₅;
R₂ is H, C₁-C₆ alkyl, benzyl, or (CH₂)ₙNMe₂;
R₃ is (CH₂)ₘ—R₆;
or R₂ and R₃, together with the nitrogen atom to which they are attached, form a 4 to 11 membered ring system, which may be monocyclic, bicyclic, or spiro-fused, wherein said ring system may further comprise one or more additional heteroatoms selected from O, N, and S, and wherein said ring system may optionally be substituted;
R₄ and R₅ are each independently selected from H, F, and CH₃;
R₆ is selected from the group consisting of 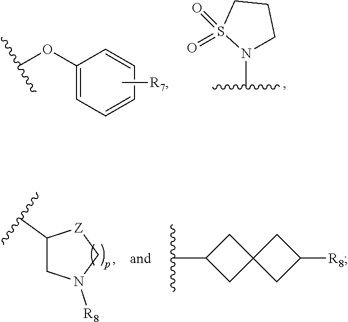
Z is CH₂, CF₂, O, S, or SO₂;
R₇ is H, halogen, OH, NH₂, C₁-C₆ alkyl, —O—C₁-C₆ alkyl, —NR₁—C₁-C₆ alkyl, —S—C₁-C₆ alkyl, or —SO₂—C₁-C₆ alkyl, wherein each said C₁-C₆ alkyl is optionally substituted with one or more F atoms;
R₈ is CO₂CH₃, SO₂R₉, or SO₂NMe₂;
R₉ is C₁-C₆ alkyl or C₃-C₆ cycloalkyl;
n is 1, 2, or 3;
m is 0, 1, 2, or 3; and
p is an integer from 0 to 3.

2. The compound of claim 1, wherein Y is CH₂ or CF₂.

3. The compound of claim 1, wherein R₁ is CH₃ or CF₃.

4. The compound of claim 1, wherein R₂ is Me, Et, Bn, or (CH₂)₂NMe₂.

5. The compound of claim 1, having a structure according to Formula Ia:

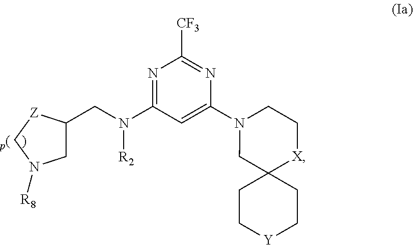

6. The compound of claim 5, wherein Z is O, S, SO₂, CH₂, or CF₂, and p is 0, 1, 2 or 3, provided that when Z is O, S, or SO₂, p is not 0 or 1.

7. The compound of claim 6, wherein Z is O.

8. The compound of claim 5, wherein Z is CH₂ and p is 0, 1, or 2.

9. The compound of claim 5, wherein R₈ is CO₂Me or SO₂NMe₂.

10. The compound of claim 5, wherein R₈ is SO₂R₉, and R₉ is Me, Et, or cyclopropyl.

11. The compound of claim 5, having a structure according to Formula Ib: 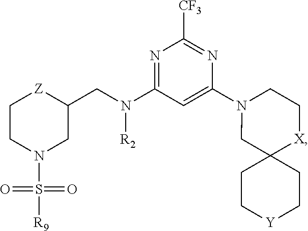
wherein:
R₂ is Me, Et, or CH₂CH₂NMe₂;
X is CH₂, CF₂, O, S, or SO₂;
Y is CH₂ or CF₂; and
Z is O, S, SO₂, or CF₂;
R₉ is Me, Et, or cyclopropyl.

12. The compound of claim 11, wherein:
X is CH₂, CF₂, O, S;
Y is CH₂; and
Z is O, S, or CF₂.

13. The compound of claim 5, wherein:
p is 3;
X is CH₂, CF₂, O, or S;
Y is CH₂;
Z is O;
R₂ is Me or Et; and
R₉ is Me, Et, or cyclopropyl.

14. The compound of claim 5, wherein:
p is 0;
X, Y, and Z are CH₂;
R₂ is Me or Et;
R₈ is SO₂R₉; and
R₉ is Me or Et.

15. The compound of claim 11, wherein:
X is O;
Y is CH₂ or CF₂;
Z is O;
R₂ is Me or Et; and
R₉ is Me, Et, or cyclopropyl;
wherein the compound has in vitro antiparasitic activity toward Dirofilaria immitis at a concentration of about 10 mM or less, or about 1 mM or less.

16. The compound of claim 5, wherein:
P is 2 or 3;
X is CH₂, CF₂, or O;
Y is CH₂ or CF₂;
Z is O CF₂, or SO₂;
R₂ is Me; and
R₉ is Me;
wherein the compound has in vitro antiparasitic activity toward Dirofilaria immitis at a concentration in a range from about 30 mM to about 100 mM.

17. The compound of claim 1, wherein R₂ and R₃, together with the nitrogen atom to which they are attached, form a 4 to 11 membered bicyclic ring system, the mono- or bicyclic ring system optionally comprising one or more heteroatoms selected from O, N, and S.

18. The compound of claim 1, wherein R₂ and R₃, together with the nitrogen atom to which they are attached, form a 7 to 11 membered spiro-fused ring system, the spiro-fused ring system optionally comprising one or more heteroatoms selected from O, N, and S.

19. A pharmaceutical composition comprising an effective amount of the compound of claim 1 and a pharmaceutically acceptable carrier, excipient, or vehicle.

20. A method for the treatment or prevention of a parasitic infection or infestation in an animal, comprising administering to the animal an effective amount of the compound of claim 1,
